(19) United States
(12) Patent Application Publication
(10) Pub. No.: US 2014/0286957 A1
(43) Pub. Date: Sep. 25, 2014

(54) ANTIBODIES TO CD1d

(71) Applicant: Teva Pharmaceuticals Australia Pty Ltd, Macquarie Park (AU)

(72) Inventors: Jonathan Kannan Nambiar, St. Ives (AU); Lynn Dorothy Poulton, Macquarie Park (AU); Adam Clarke, Five Dock (AU); Andrew James Pow, Pascoe Vale (AU); Debra Tamvakis, Camberwell (AU); George Kopsidas, Preston (AU); Anthony Gerard Doyle, Drummoyne (AU); Matthew Pollard, Dural (AU); Huseyin Mustafa, Essendon (AU)

(73) Assignee: Teva Pharmaceuticals Australia Pty Ltd, Macquarie Park (AU)

(21) Appl. No.: 14/249,566

(22) Filed: Apr. 10, 2014

Related U.S. Application Data
(63) Continuation of application No. PCT/AU2012/001247, filed on Oct. 15, 2012.
(60) Provisional application No. 61/547,307, filed on Oct. 14, 2011.

(30) Foreign Application Priority Data
Oct. 14, 2011 (AU) 2011904190

(51) Int. Cl. C07K 16/28

(52) U.S. Cl. CPC C07K 16/2803
USPC 424/139.1; 530/387.3; 530/387.9

(57) ABSTRACT

Isolated antibodies or antigen binding portions thereof that bind to CD1d. These antibodies and antigen binding portions thereof have application in treatment of conditions involving NKT cell effector function.

### CROSS REFERENCE TO RELATED APPLICATIONS

This application is a continuation of PCT/AU2012/001247, filed on Oct. 15, 2012, and claims priority to U.S. Provisional Application No. 61/547,307 filed on Oct. 14, 2011 and Australian Application No. 2011904190 filed on Oct. 14, 2011. Each application is incorporated by reference herein, in its entirety and for all purposes.

### REFERENCE TO A SEQUENCE LISTING

This application includes a Sequence Listing submitted electronically as a text file named CD1d Antibodies_ST25, created on Apr. 8, 2014, with a size of 168,000 bytes. The Sequence Listing is incorporated by reference herein.

### FIELD OF THE INVENTION

The invention relates to antibodies that bind CD1d and inhibit CD1d-mediated biological functions such as activation of the CD1d-restricted T cell, natural killer T (NKT) cells.

### BACKGROUND OF THE INVENTION

Bibliographic details of the publications referred to by the authors in this specification are collected alphabetically at the end of the description.

Reference to any prior art in this specification is not, and should not be taken as, an acknowledgment or any form of suggestion that this prior art forms part of the common general knowledge in any country.

CD1d is a counter-receptor essential for triggering cell populations, such as NKT cells, to release high levels of cytokines, an activity associated with some inflammatory diseases. Blockade of CD1d-mediated effects is therefore of potential therapeutic benefit.

CD1d protein is displayed on a number of antigen presenting cell (APC) subsets including Langerhans cells (the major dendritic antigen-presenting cells in skin), activated B-cells, dendritic cells in lymph nodes, and activated blood monocytes. One population of cells stimulated via CD1d is NKT cells, a subset of T cells that express an alpha/beta (αβ) T cell receptor (TCR) along with a variety of molecular markers typically associated with NK cells, such as CD161 and NKG2D. NKT cells are stimulated by antigen presenting cells (APC) via CD1d-presenting lipids or glycolipids. The majority of human CD1d-restricted NKT cells express a semi-invariant TCR comprising Vα24Jα18 paired with Vβ11 (Brigl, M et al., 2004 Annu. Rev. Immunol., 22:817-890). CD1d-TCR interactions rapidly induce many Th1- or Th2-like cytokines, such as interferon (IFN)-γ and tumour necrosis factor (TNF)-α, and interleukin (IL)-4, IL-5 and IL-13. The balance of Th1/Th2 cytokine responses is known to play an important role in orchestrating immune response properties.

Five CD1 genes have thus far been identified in humans: CD1a, CD1b, CD1c, CD1d and CD1e. CD1 proteins are expressed as large subunits (heavy chains) non-covalently associated with β2-microglobulin (β2M) (Van Agthoven, A., and Terhorst, C., 1982 J. Immunol. 128:426-432; Terhorst, C., et al., 1981 Cell 23:771-780). The extracellular domain of CD1d consists of three domains: the α1 domain (residues 20-108), the α2 domain (residues 109-201), and the α3 domain (residues 202-295) (Pellicci, D. G., et al., 2009 Immunity 31: 47-59).

A variety of lipids with different structures have been shown to bind CD1d molecules in a unique manner that accommodates a fatty acid chain in each of the two hydrophobic binding pockets (A′ and F) of the CD1d molecule. Lipid species capable of binding CD1d molecules include mycolic acids, diacylglycerols, sphingolipids, polyisoprenoids, lipopeptides, phosphomycoketides and small hydrophobic compounds (Venkataswamy, M. M. and Porcelli, S. A., 2010 Semin immunol 22: 68-78). The prototypical compound used to study NKT cell activation in vitro and in vivo is KRN7000, an α-galactosylceramide (“αGalCer”) derived from the marine sponge Agelas mauritianus. Additional agonists include but are not restricted to isoglobotrihexosylceramide (“iGb3”), reported to be an endogenous glycosphingolipid, as well as members of a class of microbial-derived α-glycuronosylceramides, and a variety of human glycolipids such as lysophophatidylcholine and lysosphingomyelin (Fox, L. M., et al., 2009 Plos Biol 7: e1000228). Certain naturally occurring beta-linked glycosphingolipids such as the C24:1 form of β-D-glucopyranosylceramide, are also weak agonists for NKT cells (Brennan, P. J., et al., 2011 Nat Immunol 12:1202-1211).

Excessive cytokine production by NKT cells may contribute to the pathology of certain autoimmune or inflammatory diseases such as myasthenia gravis (Reinhardt, C. et al., 1999 Neurology 52:1485-87), psoriasis (Bonish, B. D., et al., 2000 J. Immunol. 165:4076-85), ulcerative colitis (Saubermann, L. J., et al., 2000 Gastroenterology 119:119-128), primary biliary cirrhosis (Kita, H., et al., 2002 Gastroenterology 123:1031-43), colitis (Heller, F., et al. 2002 Immunity 17, 629-638), steatohepatitis (Syn, W., et al., (2010) Hepatology, 51(6):1998-2007), autoimmune hepatitis (Santodomingo-Garzon, T. and Swain, M. G. (2011) Autoimmunity Reviews 10:793-800), atherosclerosis (Kyriakakis, E., et al., Eur J Immunol 2010 40:3268-79) and pulmonary inflammation or dysfunction associated with sickle cell disease (Wallace et al. 2009 Blood 114:667-676). There is increasing evidence for NKT cells to exert detrimental effects in asthma (Iwamura, C. and Nakayama, T., 2010 Curr Opin Immunol 22:807-13).

Asthma is a chronic inflammatory pulmonary disorder characterized by reversible airway obstruction arising from chronic local inflammation, mucus obstruction, and bronchospasm in response to nonspecific stimuli (Murdoch, J. R. and Lloyd, C. M. 2010 Mutat Res 690: 24-39). The high asthma prevalence, increasing incidence and enormous associated healthcare expenditure positions asthma as a major public health problem (Holgate, S. T. and Polosa, R. 2008 Nat Rev Immunol 8: 218-30; Bahadori, K., Doyle-Waters M. M., et al., 2009 BMC Pulm Med 9:24). There is a significant unmet medical need for treatments for patients suffering from severe forms of asthma, such as corticosteroid-refractory asthma. Patients with severe asthma do not respond well to the standard-of-care and represent approximately 5-10% of the total asthmatic population. This comprises around 850,000 patients in the United States alone.

In mouse models of allergic asthma, NKT cells have been shown to exacerbate disease (Akbari, O., et al. 2003 Nat Med 9: 582-8). NKT cells may become activated by CD1d-restricted glycolipid antigens and release cytokines such as IFN-γ, IL-4, IL-5 and IL-13, which activate eosinophils and other cellular subsets important in asthma (Chuang, Y. H., et al., 2011 J Immunol 186: 4687-92). By targeting NKT cells, the administration of anti-CD1d antibodies or CD1d-dependent antagonists suppresses experimentally induced airway inflammation (Lisbonne, M., et al. 20031 Immunol 171: 1637-41; Pichavant, M., et al. 2008 J Exp Med, 205: 385-93). NKT cells are also detrimental in non-human primate models of asthma (Matangkasombut, P. et al., 20081 Allergy Clin Immunol 121: 1287-9). Such results suggest that the low numbers of NKT cells present in the lungs may be important for the development and perpetuation of human asthma.

Nonalcoholic fatty liver disease (NAFLD) is a condition in which excess fat accumulates in patients without a history of alcohol abuse. NAFLD is classified into simple steatosis and nonalcoholic steatohepatitis (NASH). In NASH, steatosis, intralobular inflammation and hepatocellular ballooning are present, often accompanied by progressive fibrosis. Long-standing NASH may progress to liver cirrhosis, and hepatocellular carcinoma (HCC) may be an outcome. NAFLD is regarded as a hepatic manifestation of metabolic syndrome. NAFLD has been increasing worldwide over recent decades in line with the increased prevalence of obesity, type 2 diabetes, and hyperlipemia. NAFLD/NASH is currently regarded as the most common chronic liver disease worldwide. It is estimated that about 20% of all adults have NAFLD and 2-3% of adults have NASH. Nonalcoholic fatty liver disease is a major cause of chronic liver disease. It encompasses a spectrum of histopathology, including hepatic steatosis (fatty liver) and nonalcoholic steatohepatitis (NASH).

The liver harbors resident populations of NKT cells which may regulate innate immune responses. For example, NKT cells with an invariant T cell receptor comprise up to 20% of T cells in murine livers. Such cells are also enriched in human livers (up to 10% of T cells) which harbor a more diverse repertoire of NKT cells. In both species, NKT cells reside mainly in the hepatic sinusoids, where they provide intravascular immune surveillance. NKT cells specifically recognize glycolipid antigens and can produce cytokines when activated. This cell subset may contribute to the pathogenesis of NASH (see for example Syn, W., et al., (2010) Hepatology, 51(6):1998-2007). Accordingly, delivery of an anti-CD1d antibody that blocks the function of NKT cells in vivo may be of therapeutic benefit.

The three main broad categories of autoimmune liver disease are autoimmune hepatitis (AIH), primary biliary cirrhosis (PBC), and primary sclerosing cholangitis (PSC). Each of these diseases has a relatively distinct clinical, serologic and histologic profile. These three liver diseases also differ in the histopathological patterns of liver injury. AIH is characterized by a progressive destruction of the hepatic parenchyma, known as interface hepatitis. On the other hand, PBC is distinguished by specific destruction of small intra hepatic bile ducts, whereas PSC mainly involves destruction of large bile ducts. Despite the varied profiles of these conditions all these autoimmune hepatic diseases share common pathways of immune-mediated liver injury involving the hepatic recruitment of T lymphocytes which recognize and destroy hepatocytes, with the subsequent development of liver fibrosis. NKT cells may contribute to the pathology of autoimmune liver diseases (Santodomingo-Garzon, T. and Swain, M. G. (2011) Autoimmunity Reviews 10:793-800). Activated NKT cells may induce hepatocyte death directly through up-regulation of cell surface FasL expression and/or the release of tumor necrosis factor alpha (TNF-α) and perforins/granzyme B. NKT cells may indirectly induce hepatocyte death through the release of pro-inflammatory cytokines such as IFN-γ. NKT cells can also produce IL-4, which induces Th2 responses and the subsequent production of autoantibodies by plasma cells. Since activation of NKT cells can lead to hepatocyte destruction and ultimately the development of cirrhosis, the blockade of NKT cell function by delivery of an anti-CD1d antibody may therefore be of therapeutic benefit. In addition to cytokine release, NKT cell effector functions which result in cell lysis, such as perforin release and granzyme release and Fas-L mediated cell death, and other known NKT functions such as IL-2 mediated bystander effects, may also be relevant in conditions in which NKT cells are implicated. Blockade of the NKT cell activator CD1d, for example through administration of an anti-CD1d antibody, may also modulate these NKT effector functions.

### SUMMARY OF THE INVENTION

There is a critical need to identify therapies that inhibit CD1d-mediated cell activation and subsequently show benefit in the treatment of inflammatory diseases such as severe corticosteroid refractory asthma. Fully human antibodies possess several advantages to address the goal of developing human medicines that improve therapeutic efficacy. They can be targeted to bind highly potent neutralizing epitopes and when administered to humans are well-tolerated. While murine antibodies have been described in the art that bind and interact with CD1d, the current invention describes human antibodies that exhibit strong potency in inhibiting CD1d mediated NKT cell activation and resultant effector function. Surprisingly, in some cases the potency of these antibodies is orders of magnitude more potent than current state of the art antibodies. Such antibodies with significantly enhanced potency should allow for the treatment of CD1d-mediated diseases and should exhibit superior clinical efficacy.

Accordingly in a first aspect the invention provides an isolated antibody or antigen binding portion thereof which binds to human CD1d wherein the isolated antibody or antigen binding portion thereof competes for binding to CD1d with at least one antibody selected from the group consisting of 401.11 and 402.8.

In a second aspect the invention provides an isolated antibody or antigen binding portion thereof which binds to human CD1d wherein the isolated antibody or antigen binding portion thereof binds to the same epitope of CD1d as that bound by at least one antibody selected from the group consisting of 401.11 and 402.8.

In a third aspect the invention provides an isolated antibody or antigen binding portion thereof which binds to human CD1d wherein the isolated antibody or antigen binding portion thereof comprises a VH domain having a sequence selected from the group consisting of SEQ ID NOs 1, 3, 5, 7, 8, 9, 24, 25, 26, 30, 33, 36, 40, 41, 42, 43, 44 and 45 and sequences at least 95% identical thereto.

In a fourth aspect the invention provides an isolated antibody or antigen binding portion thereof which binds to human CD1d wherein the isolated antibody or antigen binding portion thereof comprises a VL domain having a sequence selected from the group consisting of SEQ ID NOs 2, 4, 6, 46, 49 and 62 and sequences at least 95% identical thereto.

In a fifth aspect the invention provides an isolated antibody or antigen binding portion thereof which binds to human CD1d wherein the isolated antibody or antigen binding portion thereof comprises a VH domain comprising human FR1, FR2, FR3 and FR4 framework sequences and CDR1, CDR2 and CDR3 sequences and wherein the sequence of CDR1 is DYAMH (SEQ ID NO: 124) or GYYWS (SEQ ID NO: 125).

In a sixth aspect the invention provides an isolated antibody or antigen binding portion thereof which binds to human CD1d wherein the isolated antibody or antigen binding portion thereof comprises a VH domain comprising human FR1, FR2, FR3 and FR4 framework sequences and CDR1, CDR2 and CDR3 sequences and wherein the sequence of CDR1 is GFTFDDY (SEQ ID NO: 135) or GGSFSGY (SEQ ID NO: 136).

In a seventh aspect the invention provides an isolated antibody or antigen binding portion thereof which binds to human CD1d wherein the isolated antibody or antigen binding portion thereof comprises a VL domain comprising human FR1, FR2, FR3 and FR4 framework sequences and CDR1, CDR2 and CDR3 sequences and wherein the sequence of CDR1 is RASQHISSWLA (SEQ ID NO: 141) or ASSSGAVSSGNFPN (SEQ ID NO: 142).

In an eighth aspect the invention provides an isolated antibody or antigen binding portion thereof which binds to human CD1d wherein the isolated antibody or antigen binding portion thereof binds to CD1d with an EC50 of less than 20 ng/ml as measured using a cell based potency assay. In one embodiment the isolated antibody or antigen binding portion thereof binds to human CD1d with an EC50 of from 0.5 ng/ml to 20 ng/ml.

In a ninth aspect the invention provides an isolated DNA molecule which encodes the isolated antibody or antigen binding portion thereof of the invention.

In a tenth aspect the invention provides a method of treating a condition involving NKT cell effector function in a human subject comprising administering to the subject an isolated antibody or antigen binding portion thereof of the invention.

In an eleventh aspect the invention provides a method of detecting the presence of CD1d in a sample the method comprising contacting a sample suspected to contain CD1d with the isolated antibody or antigen binding portion thereof of the invention under conditions which allows the binding of the antibody or antigen binding portion thereof to CD1d to form a complex and detecting the presence the complex in the sample.

In a twelfth aspect the invention provides a method of detecting the presence of CD1d-positive cells in a cell sample the method comprising contacting a population of cells with an isolated antibody or antigen binding portion thereof of the invention to allow the binding of the antibody or antigen binding portion thereof to CD1d-positive to form a complex and detecting the presence of the antibody or antigen binding portion thereof-cell complex.

In a thirteenth aspect the invention provides a method of selecting a CD1d-binding protein which binds specifically to human CD1d and competes for binding on CD1d with at least one antibody selected from the group consisting of 401.11, 402.8 and 401.11.158 from a plurality of CD1d-binding proteins, the method comprising:
contacting the plurality of CD1d-binding proteins to a human CD1d mutein in which the amino acid positions 87 to 93 and 141-143 of SEQ ID NO: 116 have been substituted with corresponding murine amino acids at these positions, under conditions sufficient to allow binding of CD1d-binding proteins to the mutein to form a CD1d-binding protein-human CD1d mutein complex and a depleted plurality of CD1d-binding proteins which do not bind the human CD1d mutein, and
collecting CD1d-binding proteins which do not bind to the human CD1d mutein from the depleted plurality of CD1d-binding proteins,
wherein the collected CD1d-binding proteins bind specifically to human CD1d and compete for binding on CD1d with at least one antibody selected from the group consisting of 401.11, 402.8 and 401.11.158.

In a fourteenth aspect the invention provides a method of selecting a CD1d-binding protein which binds specifically to CD1d from a plurality of CD1d-binding proteins, the method comprising:
contacting the plurality of CD1d-binding proteins to hCD1dmu (SEQ ID NO: 119) in which the amino acids located at positions 87 to 93 and 141 to 143 of human CD1d (SEQ ID NO 116) have been replaced with the corresponding murine sequence at these positions, under conditions sufficient to allow binding of CD1d-binding proteins to the hCD1dmu to form a CD1d-binding protein-hCD1dmu complex and a depleted plurality of CD1d binding proteins which do not bind hCD1dmu, and
collecting CD1d-binding proteins which do not bind to the hCD1dmu from the depleted plurality of CD1d-binding proteins,
wherein the collected CD1d binding proteins bind specifically to human CD1d (SEQ ID NO: 116) or mCD1dhu (SEQ ID NO: 118).

In one embodiment of any of the above aspects the isolated antibody or antigen binding portion thereof also binds to cynomolgus and rhesus monkey CD1d.

### BRIEF DESCRIPTION OF THE DRAWINGS

FIG. 1 shows a graphical representation of results of an assay demonstrating inhibition of tetramer binding by anti-CD1d antibodies. Anti-CD1d antibodies 401.11 and 402.8 showed improved inhibition of CD1d tetramer binding compared with antibodies 42 and 51.1, as determined by a reduction in the mean fluorescence intensity of the signal, in an assay using a α-Galactosylceramide (α-GalCer) lipid-loaded); CD1d tetramer binding to J.RT3-T3.5 cells stably transfected with an NKT cell receptor. The irrelevant specificity negative control antibody showed no inhibition. Table 2 lists the EC₅₀ values of all antibodies tested.

FIG. 2 shows a graphical representation of results of an assay demonstrating inhibition of IL-2 release by anti-CD1d antibodies. Anti-CD1d antibodies 402.8 and 401.11 showed improved inhibition of IL-2 release after 24 hours, as determined by ELISA, compared with anti-CD1d antibodies 42 and 51.1 in an assay using α-GalCer-loaded CD1d-positive U-937 cells and NKT cell receptor-stably transfected J.RT3-T3.5 cells. In all assays the irrelevant specificity negative control antibody showed no inhibition of IL-2 release. EC₅₀ values from representative experiments are presented in Table 3.

FIG. 3 shows a graphical representation of results of an assay demonstrating binding of anti-CD1d antibodies to Primary peripheral blood mononuclear cells (PBMCs) by flow cytometry. Anti-CD1d antibody 402.8, as an example of antibodies described in this specification, but not an irrelevant specificity negative control antibody, bound a CD1d-positive, CD11c-positive population in primary human PBMCs, as determined by flow cytometry.

FIG. 4 shows a graphical representation of results of an assay demonstrating inhibition of primary NKT cell function by anti-CD1d antibodies in Primary NKT Cell-Based Assays using THP-1 cell line as Antigen-Presenting Cells. Antibodies 401.11 and 402.8 exhibited up to 114-fold and up to 180-fold improved inhibition respectively of IFN-γ (A), IL-4 (B), IL-5 (C) and IL-13 (D) release after 24 hours, as determined by ELISA, compared with anti-CD1d antibody 42. This result was from an assay using α-GalCer-expanded NKT cells and α-GalCer-loaded THP-1 cells as CD1d-positive cells. In all assays, the irrelevant specificity negative control antibody did not inhibit cytokine release. EC₅₀ values from representative experiments are presented in Table 4.

FIG. 5 shows a graphical representation of results of an assay demonstrating inhibition of primary NKT cell function by anti-CD1d antibodies in Primary NKT Cell-Based Assays using primary CD14+ monocytes as Antigen Presenting Cells. Antibodies 401.11 and 402.8 demonstrated significantly improved inhibition of IFN-γ (A), IL-4 (B), IL-5 (C) and IL-13 (D) release after 24 hours, as determined by ELISA, compared with anti-CD1d antibodies 42 and 51.1 in an assay using α-GalCer-expanded NKT cells and α-GalCer-loaded CD14+ monocyte-derived dendritic cells as CD1d-positive cells. In all assays, the irrelevant specificity negative control antibody did not inhibit cytokine release. EC₅₀ values from representative experiments are presented in Table 5.

FIG. 6 shows a graphical representation of results of a competition ELISA demonstrating that highly potent anti-CD1d antibodies share a similar neutralizing epitope that is different to the epitope seen by lower-potency prior-art antibodies. As per Example 7, anti-CD1d antibody 402.8 competed with itself and with 401.11, but not with anti-CD1d antibodies 42 and 51.1, for binding to human CD1d using a competition ELISA based approach, as shown by absorbance readings at 450 nm corresponding to the levels of bound biotinylated 402.8 (A) and converted degree of competition (percentage) values (B).

FIG. 7 shows a graphical representation of results of an assay demonstrating cross-reactivity with recombinant cynomolgus macaque CD1d. As per Example 8, anti-CD1d antibodies 401.11 and 402.8 bound human CD1d (A) and were cross-reactive with cynomolgus macaque CD1d (B) by ELISA.

FIG. 8 shows a graphical representation of results of an assay demonstrating cross-reactivity with cynomolgus macaque cell-based CD1d. As per Example 9, anti-CD1d antibody 402.8, but not an irrelevant specificity negative control antibody, bound CD1d on PBMCs from two independent cynomolgus macaque donors as shown by flow cytometry. Data are presented as flow cytometry histograms of gated live cells with the percentage of CD1d-positive cells demarcated in the histogram.

FIG. 9 shows a graphical representation of results of an assay demonstrating cell-based inhibition of cynomolgus CD1d-mediated primary NKT expansion. As per Example 10, anti-CD1d antibody 402.8, but not an irrelevant specificity negative control antibody, inhibited the expansion of cynomolgus NKT cells in the presence of αGalCer-loaded CD1d-positive PBMCs, as shown by quantification of CD3+Vα24+ cells by flow cytometry.

FIG. 10 shows a sequence alignment showing sequences of variable regions of 401.11. Boxed regions contain CDRs (as indicated) as defined by the Kabat numbering system and the enhanced Chothia numbering system. CDRs defined by the Kabat numbering system are shown in bold. CDRs defined by the enhanced Chothia numbering system are underlined.

FIG. 11: shows a sequence alignment showing sequences of variable regions of 402.8. Boxed regions contain CDRs (as indicated) as defined by the Kabat numbering system and the enhanced Chothia numbering system. CDRs defined by the Kabat numbering system are shown in bold. CDRs defined by the enhanced Chothia numbering system are underlined.

FIGS. 12A and 12B show an alignment of variants of 401.11. As per Example 11, an amino acid sequence alignment of the heavy (FIG. 12A) and light chains (FIG. 12B) of 401.11 versus IGHV3-9.01 and variants of 401.11 is presented.

FIGS. 13A and 13B show an alignment of optimized variants of 401.11. As per Example 11, an amino acid sequence alignment of the heavy (FIG. 13A) and light chains (FIG. 13B) of 401.11 and variants thereof is presented.

FIG. 14 shows a graphical representation of results of an assay demonstrating improved inhibition of primary NKT cell function by enhanced variants of anti-CD1d antibody 401.11. As per Example 11, 401.11 and variants thereof were titrated from 1 μg/mL. 401.11 antibody variants demonstrated similar or improved inhibition of IFN-γ (A) and IL-4 (B) release after 24 hours, as determined by ELISA, compared with 401.11, and significantly improved inhibition of IFN-γ (A) and IL-4 (B) release after 24 hours, as determined by ELISA, compared with anti-CD1d antibodies 42 and 51.1 titrated from 10 μm/mL, in an assay using α-GalCer-expanded NKT cells and α-GalCer-loaded CD14+ monocyte-derived dendritic cells as CD1d-positive cells. In all assays, the irrelevant specificity negative control antibody did not inhibit cytokine release. EC₅₀ values from representative experiments are presented in Table 13.

FIG. 15 shows an alignment of optimized variants of 402.8. As per Example 11, an amino acid sequence alignment of the heavy chain of 402.8 versus variants of 402.8 is presented.

FIG. 16 shows a graphical representation of results of an assay demonstrating inhibition of primary NKT cell function by enhanced variants of anti-CD1d antibody 402.8. As per Example 11, 402.8 and variants thereof were titrated from 10 μg/mL and demonstrated similar inhibition of IFN-γ (A) and IL-13 (B) release after 24 hours, as determined by ELISA, and significantly improved inhibition of IFN-γ (A) and IL-13 (B) release after 24 hours, as determined by ELISA, compared with anti-CD1d antibody 42 titrated from 10 μg/mL, in an assay using α-GalCer-expanded NKT cells and α-GalCer-loaded CD14+ monocyte-derived dendritic cells as CD1d-positive cells. In all assays, the irrelevant specificity negative control antibody did not inhibit cytokine release. EC₅₀ values from representative experiments are presented in Table 18.

FIG. 17 shows a graphical representation of results of an assay demonstrating improved inhibition of primary NKT cell function by anti-CD1d antibodies in Primary NKT cell-based assays using an alternative antigen to α-GalCer. As per Example 12, antibodies 401.11.158, 401.11 and 402.8 titrated from 1 μg/mL, demonstrated significantly improved inhibition of IFN-γ (A) and IL-4 (B) release after 24 hours, as determined by ELISA, compared with anti-CD1d antibodies 42 and 51 titrated from 10 m/mL, in an assay using α-GalCer-expanded NKT cells and C24:1 β-D-glucopyranosylceramide-loaded CD14+ monocyte-derived dendritic cells as CD1d-positive cells. In all assays, the irrelevant specificity negative control antibody did not inhibit cytokine release. EC₅₀ values from representative experiments are presented in Table 20.

FIG. 18 shows a graphical representation of results of a competition ELISA demonstrating that under revised conditions, highly potent anti-CD1d antibodies share a similar neutralizing epitope that is different to the epitope seen by prior-art antibodies. As per Example 13, antibody 402.8 competed with itself and with 401.11, but not with antibodies 42 and 51.1, for binding to human CD1d, as shown by absorbance readings at 450 nm (A) and converted degree of competition (percentage) values (B).

FIG. 19 shows a graphical representation of results of a competition ELISA demonstrating that highly potent anti-CD1d antibodies which were variants of 401.11 shared a similar neutralizing epitope with 402.8. As per Example 13, anti-CD1d antibody 402.8 competed strongly with itself and with 401.11.160, 401.11.161 and 401.11.165 as examples of 401.11 antibody variants for binding to human CD1d, as shown by absorbance readings at 450 nm (A) and converted degree of competition (percentage) values (B).

FIG. 20 shows a graphical representation of results of a competition ELISA demonstrating that highly potent anti-CD1d antibodies derived from 402.8 share a similar neutralizing epitope with 402.8. As per Example 13, anti-CD1d antibody 402.8 competed strongly with itself and with 402.8.84, 402.8.86 and 402.8.87, as examples of 402.8 antibody variants for binding to human CD1d, as shown by absorbance readings at 450 nm (A) and converted degree of competition (percentage) values (B).

FIG. 21 shows a graphical representation of results of a Competition ELISA demonstrating that monoclonal anti-human CD1d antibodies do not compete with the neutralizing epitope of 402.8. As described in Example 13, anti-CD1d antibody 402.8 competed strongly with itself but not with other monoclonal anti-human CD1d antibodies, such as AD58E7, C3D5 and C-9, for binding to human CD1d, as shown by absorbance readings (A) and converted degree of competition (percentage) values (B).

FIG. 22 shows a graphical representation of results of a competition ELISA demonstrating that monoclonal anti-mouse CD1d antibodies do not compete for the neutralizing epitope of 402.8. As described in Example 13, anti-CD1d antibody 402.8 competed strongly with itself but not with monoclonal anti-mouse CD1d antibodies, such as HB-321, HB-322 and HB-323, for binding to human CD1d, as shown by absorbance readings at 450 nm (A) and converted degree of competition (percentage) values (B).

FIG. 23 shows a graphical representation of results of a competition ELISA demonstrating that polyclonal anti-human CD1d antibodies do not compete for the neutralizing epitope of 402.8. As described in Example 13, anti-CD1d antibody 402.8 competed strongly with itself but not with C-19, H70 and Ab96515, as examples of polyclonal anti-human CD1d antibodies, for binding to human CD1d, as shown by absorbance readings at 450 nm (A) and converted degree of competition (percentage) values (B).

FIG. 24 shows a graphical representation of results of a competition ELISA demonstrating that highly potent anti-CD1d antibodies share a similar neutralizing epitope that is different to the epitopes bound by other anti-CD1d antibodies. As described in Example 13, anti-CD1d antibody 401.11.158 competed strongly with itself and with 402.8, but not with anti-CD1d antibodies 42 and 51.1, for binding to human CD1d, as shown by absorbance readings at 450 nm (A) and converted degree of competition (percentage) values (B).

FIG. 25 shows a graphical representation of results of an ELISA demonstrating that 402.8, and 401.11.165 in the form of a FAb or a full length IgG bound to human CD1d.

FIG. 26 shows a sequence alignment of CD1d constructs used to elucidate the location on human CD1d to which the anti-CD1d antibodies bind.

FIG. 27 shows a graphical representation of results of an ELISA demonstrating that a titration of antibodies 402.8 (A) and 401.11.158 (B) bound to human CD1d and mouse CD1d into which human sequence had been introduced (mCD1dhu). Both antibodies did not bind to mouse CD1d or a human CD1d into which mouse sequence had been introduced (hCD1dmu).

FIG. 28 shows a graphical representation of results of hydrogen-deuterium exchange mapping experiments demonstrating the epitope of anti-human CD1d antibodies. (A) Human CD1d (grey) with amino acid 89-94 and 141-142 indicated in black. Note: X-ray structure is 3HUJ with a surface representation. (B) Human CD1d (with α-GalCer bound) in complex with the NKT-cell receptor (α and β chains). The atoms of the epitope (amino acids 89-94 and 141-142) of the anti-CD1d antibodies on human CD1d are coloured dark grey. The epitope of the anti-CD1d antibodies is located in close proximity to the binding site of the NKT-cell receptor β-chain.

FIGS. 29A and 29B show an alignment and consensus sequence of the V_{H} region of optimized 401.11 antibodies. Boxed regions contain CDRs (as indicated) as defined by the Kabat numbering system and the enhanced Chothia numbering system. CDRs defined by the Kabat numbering system are shown in bold. CDRs defined by the enhanced Chothia numbering system are underlined.

FIGS. 29C and 29D show an alignment and consensus sequence of the V_{L} region of optimized 401.11 antibodies. Boxed regions contain CDRs (as indicated) as defined by the Kabat numbering system and the enhanced Chothia numbering system. CDRs defined by the Kabat numbering system are shown in bold. CDRs defined by the enhanced Chothia numbering system are underlined.

FIG. 30A shows an alignment and consensus sequence of the V_{H} region of optimized 402.8 antibodies. Boxed regions contain CDRs (as indicated) as defined by the Kabat numbering system and the enhanced Chothia numbering system. CDRs defined by the Kabat numbering system are shown in bold. CDRs defined by the enhanced Chothia numbering system are underlined. FIG. 30B shows an alignment and consensus sequence of the V_{L} region of optimized 402.8 antibodies. Boxed regions contain CDRs (as indicated) as defined by the Kabat numbering system and the enhanced Chothia numbering system. CDRs defined by the Kabat numbering system are shown in bold. CDRs defined by the enhanced Chothia numbering system are underlined.

### DETAILED DESCRIPTION OF THE INVENTION

The invention relates to human and humanized antibodies and antigen binding portions thereof which bind a particular epitope of CD1d. The present inventors have found that antibodies which bind this epitope of CD1d are particularly efficacious in decreasing the effect of CD1d on NKT cells. Due to this effect it is believed that these antibodies and antigen binding portions thereof will be useful in the treatment of conditions in which NKT cell effector function, such as excessive production of cytokines by NKT cells plays a role, such as asthma.

Accordingly in a first aspect the invention provides an isolated antibody or antigen binding portion thereof which binds to human CD1d wherein the isolated antibody or antigen binding portion thereof competes for binding to CD1d with at least one antibody selected from the group consisting of 401.11 and 402.8.

In a second aspect the invention provides an isolated antibody or antigen binding portion thereof which binds to human CD1d wherein the isolated antibody or antigen binding portion thereof binds to the same epitope of CD1d as that bound by at least one antibody selected from the group consisting of 401.11 and 402.8.

In a third aspect the invention provides an isolated antibody or antigen binding portion thereof which binds to human CD1d wherein the isolated antibody or antigen binding portion thereof comprises a VH domain having a sequence selected from the group consisting of SEQ ID NOs: 1, 3, 5, 7, 8, 9, 24, 25, 26, 30, 33, 36, 40, 41, 42, 43, 44 and 45 and sequences at least 95% identical thereto.

In a fourth aspect the invention provides an isolated antibody or antigen binding portion thereof which binds to human CD1d wherein the isolated antibody or antigen binding portion thereof comprises a VL domain having a sequence selected from the group consisting of SEQ ID NOs: 2, 4, 6, 46, 49 and 62 and sequences at least 95% identical thereto.

In a fifth aspect the invention provides an isolated antibody or antigen binding portion thereof which binds to human CD1d wherein the isolated antibody or antigen binding portion thereof comprises a VH domain comprising human FR1, FR2, FR3 and FR4 framework sequences and CDR1, CDR2 and CDR3 sequences and wherein the sequence of CDR1 is DYAMH (SEQ ID NO: 124) or GYYWS (SEQ ID NO: 125).

In an embodiment of this aspect of the invention the sequence of CDR3 is DMCSSSGCPDGYFDS (SEQ ID NO: 126), DLCSSGGCPEGYFDS (SEQ ID NO: 152), DMCSSGGCPDGYFDS (SEQ ID NO: 153), DMCSSGGCPEGYFDS (SEQ ID NO: 154), GEIYDFWNSYMDV (SEQ ID NO: 127), GEIYDFWKSYMDV (SEQ ID NO: 128), GEIYDFYKSYLDV (SEQ ID NO: 155), GEIYDFYKSYMDV (SEQ ID NO: 156), GEIYDFWKSYLDV (SEQ ID NO: 129) or GEIYDFYNSYMDV (SEQ ID NO: 130). In a further embodiment the sequence of CDR2 is TIIWNSAIIGYADSVKG (SEQ ID NO: 131), EINHSGSTNYNPSLKS (SEQ ID NO: 132), EINPSGSTNYNPSLKS (SEQ ID NO: 133) or EINHAGSTNYNPSLKS (SEQ ID NO: 134).

In a sixth aspect the invention provides an isolated antibody or antigen binding portion thereof which binds to human CD1d wherein the isolated antibody or antigen binding portion thereof comprises a VH domain comprising human FR1, FR2, FR3 and FR4 framework sequences and CDR1, CDR2 and CDR3 sequences and wherein the sequence of CDR1 is GFTFDDY (SEQ ID NO: 135) or GGSFSGY (SEQ ID NO: 136).

In an embodiment of the sixth aspect of the invention the sequence of CDR3 is DMCSSSGCPDGYFDS (SEQ ID NO: 126), DLCSSGGCPEGYFDS (SEQ ID NO: 152), DMCSSGGCPDGYFDS (SEQ ID NO: 153), DMCSSGGCPEGYFDS (SEQ ID NO: 154), GEIYDFWNSYMDV (SEQ ID NO: 127), GEIYDFWKSYMDV (SEQ ID NO: 128), GEIYDFYKSYLDV (SEQ ID NO: 155), GEIYDFYKSYMDV (SEQ ID NO: 156), GEIYDFWKSYLDV (SEQ ID NO: 129) or GEIYDFYNSYMDV (SEQ ID NO: 130). In a further embodiment the sequence of CDR2 is IWNSAI (SEQ ID NO: 137), NHSGS (SEQ ID NO: 138), NPSGS (SEQ ID NO: 139) or NHAGS (SEQ ID NO: 140).

In a seventh aspect the invention provides an isolated antibody or antigen binding portion thereof which binds to human CD1d wherein the isolated antibody or antigen binding portion thereof comprises a VL domain comprising human FR1, FR2, FR3 and FR4 framework sequences and CDR1, CDR2 and CDR3 sequences and wherein the sequence of CDR1 is RASQHISSWLA (SEQ ID NO: 141) or ASSSGAVSSGNFPN (SEQ ID NO: 142).

In an embodiment of the seventh aspect of the invention the sequence of CDR3 is QQANRFPLT (SEQ ID NO: 141) or LLYFGDTQLGV (SEQ ID NO: 142). In a further embodiment the sequence of CDR2 is AASSLQS (SEQ ID NO: 145) or SASNKHS (SEQ ID NO: 146).

In an eighth aspect the invention provides an isolated antibody or antigen binding portion thereof which binds to human CD1d wherein the isolated antibody or antigen binding portion thereof binds to CD1d with an EC50 of less than 20 ng/ml as measured using a cell based potency assay. In an embodiment of the invention the isolated antibody or antigen binding portion thereof binds to human CD1d with an EC50 of from 0.5 ng/ml to 20 ng/ml as measured using a cell based potency assay.

In an embodiment of the invention there is provided an isolated antibody or antigen binding portion thereof comprising a VH and VL sequence pair of SEQ ID NO: 1 and SEQ ID NO: 2.

In an embodiment of the invention there is provided an isolated antibody or antigen binding portion thereof comprising a VH and VL sequence pair of SEQ ID NO: 23 and SEQ ID NO: 46.

In an embodiment of the invention there is provided an isolated antibody or antigen binding portion thereof comprising a VH and VL sequence pair of SEQ ID NO: 24 and SEQ ID NO: 47.

In an embodiment of the invention there is provided an isolated antibody or antigen binding portion thereof comprising a VH and VL sequence pair of SEQ ID NO: 5 and SEQ ID NO: 6.

In an embodiment of the invention there is provided an isolated antibody or antigen binding portion thereof comprising a VH and VL sequence pair of SEQ ID NO: 25 and SEQ ID NO: 48.

In an embodiment of the invention there is provided an isolated antibody or antigen binding portion thereof comprising a VH and VL sequence pair of SEQ ID NO: 26 and SEQ ID NO: 49

In an embodiment of the invention there is provided an isolated antibody or antigen binding portion thereof comprising a VH and VL sequence pair of SEQ ID NO: 27 and SEQ ID NO: 50.

In an embodiment of the invention there is provided an isolated antibody or antigen binding portion thereof comprising a VH and VL sequence pair of SEQ ID NO: 28 and SEQ ID NO: 51.

In an embodiment of the invention there is provided an isolated antibody or antigen binding portion thereof comprising a VH and VL sequence pair of SEQ ID NO: 29 and SEQ ID NO: 52.

In an embodiment of the invention there is provided an isolated antibody or antigen binding portion thereof comprising a VH and VL sequence pair of SEQ ID NO: 30 and SEQ ID NO: 53.

In an embodiment of the invention there is provided an isolated antibody or antigen binding portion thereof comprising a VH and VL sequence pair of SEQ ID NO: 31 and SEQ ID NO: 54.

In an embodiment of the invention there is provided an isolated antibody or antigen binding portion thereof comprising a VH and VL sequence pair of SEQ ID NO: 32 and SEQ ID NO: 55.

In an embodiment of the invention there is provided an isolated antibody or antigen binding portion thereof comprising a VH and VL sequence pair of SEQ ID NO: 33 and SEQ ID NO: 56.

In an embodiment of the invention there is provided an isolated antibody or antigen binding portion thereof comprising a VH and VL sequence pair of SEQ ID NO: 34 and SEQ ID NO: 57.

In an embodiment of the invention there is provided an isolated antibody or antigen binding portion thereof comprising a VH and VL sequence pair of SEQ ID NO: 35 and SEQ ID NO: 58.

In an embodiment of the invention there is provided an isolated antibody or antigen binding portion thereof comprising a VH and VL sequence pair of SEQ ID NO: 36 and SEQ ID NO: 59.

In an embodiment of the invention there is provided an isolated antibody or antigen binding portion thereof comprising a VH and VL sequence pair of SEQ ID NO: 37 and SEQ ID NO: 60.

In an embodiment of the invention there is provided an isolated antibody or antigen binding portion thereof comprising a VH and VL sequence pair of SEQ ID NO: 38 and SEQ ID NO: 61.

In an embodiment of the invention there is provided an isolated antibody or antigen binding portion thereof comprising a VH and VL sequence pair of SEQ ID NO: 40 and SEQ ID NO: 62.

In an embodiment of the invention there is provided an isolated antibody or antigen binding portion thereof comprising a VH and VL sequence pair of SEQ ID NO: 41 and SEQ ID NO: 63.

In an embodiment of the invention there is provided an isolated antibody or antigen binding portion thereof comprising a VH and VL sequence pair of SEQ ID NO: 42 and SEQ ID NO: 64.

In an embodiment of the invention there is provided an isolated antibody or antigen binding portion thereof comprising a VH and VL sequence pair of SEQ ID NO: 3 and SEQ ID NO: 4.

In an embodiment of the invention there is provided an isolated antibody or antigen binding portion thereof comprising a VH and VL sequence pair of SEQ ID NO: 7 and SEQ ID NO: 4.

In an embodiment of the invention there is provided an isolated antibody or antigen binding portion thereof comprising a VH and VL sequence pair of SEQ ID NO: 8 and SEQ ID NO: 4.

In an embodiment of the invention there is provided an isolated antibody or antigen binding portion thereof comprising a VH and VL sequence pair of SEQ ID NO: 9 and SEQ ID NO: 4.

In an embodiment of the invention there is provided an isolated antibody or antigen binding portion thereof comprising a VH and VL sequence pair of SEQ ID NO: 43 and SEQ ID NO: 65.

In an embodiment of the invention there is provided an isolated antibody or antigen binding portion thereof comprising a VH and VL sequence pair of SEQ ID NO: 44 and SEQ ID NO: 66.

In an embodiment of the invention there is provided an isolated antibody or antigen binding portion thereof comprising a VH and VL sequence pair of and SEQ ID NO: 45 and SEQ ID NO: 67.

In an embodiment of any one of the above aspects, the antibody or antigen binding portion thereof binds to human CD1d (SEQ ID NO:116), but not to hCD1dmu (SEQ ID NO:119). In an embodiment of any of the above aspects, the antibody or antigen binding portion thereof binds to mCD1dhu (SEQ ID NO:118) but not to mCD1d (SEQ ID NO:117).

In a ninth aspect the invention provides an isolated DNA molecule which encodes the isolated antibody or antigen binding portion thereof of the invention. In one embodiment, the isolated DNA molecule is selected from any one of SEQ ID NOS: 10, 11, 12, 13, 14, 15, 16, 17, 18, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115 or a sequence at least 95% identical thereto or a sequence which hybridises thereto under moderate to high stringency conditions. In one embodiment, the isolated DNA molecule is selected from any one of SEQ ID NOS: 10, 11, 12, 13, 14, 15, 16, 17, 18, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114 or 115.

In a tenth aspect the invention provides a method of treating a condition involving NKT cell effector function in a human subject comprising administering to the subject an isolated antibody or antigen binding portion thereof of the invention.

In an eleventh aspect the invention provides a method of detecting the presence of CD1d in a sample the method comprising contacting a sample suspected to contain CD1d with the isolated antibody or antigen binding portion thereof of the invention under conditions which allows the binding of the antibody or antigen binding portion thereof to CD1d to form a complex and detecting the presence the complex in the sample.

In a twelfth aspect the invention provides a method of detecting the presence of CD1d-positive cells in a cell sample the method comprising contacting a population of cells with an isolated antibody or antigen binding portion thereof of the invention to allow the binding of the antibody or antigen binding portion thereof to CD1d-positive to form a complex and detecting the presence of the antibody or antigen binding portion thereof cell complex.

In a thirteenth aspect the invention provides a method of selecting a CD1d-binding protein which binds specifically to human CD1d and competes for binding on CD1d with at least one antibody selected from the group consisting of 401.11, 402.8 and 401.11.158 from a plurality of CD1d-binding proteins, the method comprising:
contacting the plurality of CD1d-binding proteins to a human CD1d mutein in which the amino acid positions 87 to 93 and 141-143 of SEQ ID NO: 116 have been substituted with corresponding murine amino acids at these positions, under conditions sufficient to allow binding of CD1d-binding proteins to the mutein to form a CD1d-binding protein-human CD1d mutein complex and a depleted plurality of CD1d-binding proteins which do not bind the human CD1d mutein, and
collecting CD1d-binding proteins which do not bind to the human CD1d mutein from the depleted plurality of CD1d-binding proteins, wherein the collected CD1d-binding proteins bind specifically to human CD1d and compete for binding on CD1d with at least one antibody selected from the group consisting of 401.11, 402.8 and 401.11.158.

In a fourteenth aspect the invention provides a method of selecting a CD1d-binding protein which binds specifically to CD1d from a plurality of CD1d-binding proteins, the method comprising:
contacting the plurality of CD1d-binding proteins to hCD1dmu (SEQ ID NO: 119) in which the amino acids located at positions 87 to 93 and 141 to 143 of human CD1d (SEQ ID NO 116) have been replaced with the corresponding murine sequence at this location, under conditions sufficient to allow binding of CD1d-binding proteins to the hCD1dmu to form a CD1d-binding protein-hCD1dmu complex and a depleted plurality of CD1d binding proteins which do not bind hCD1dmu and collecting CD1d-binding proteins which do not bind to the hCD1dmu from the depleted plurality of CD1d-binding proteins, wherein the collected CD1d binding proteins bind specifically to human CD1d (SEQ ID NO: 116) or mCD1dhu (SEQ ID NO: 118).

The anti-CD1d antibodies of the invention may also be used to identify or select CD1d-positive cell populations from blood. Anti-CD1d antibody may be used to detect a population of CD1d-positive cells within the peripheral blood of a human patient, including myeloid cells such as monocytes, or lymphoid cells such as B cells. The antibody could be used to detect these cells in conditions where such CD1d-positive cells contribute to disease, e.g. certain leukemias including chronic lymphocytic leukemia (CLL). (Metelitsa et al., Leukemia (2003) 17, 1068-1077.; Kotsianidis et al., 2011; Am J Clin Path 136, 400-408.)

The anti-human CD1d antibody could also be used to stain tissue sections for immunohistochemistry using methods well known in the art.

In certain embodiments of the invention the isolated antibody or antigen binding portion thereof may comprises a human kappa chain constant region or a human lambda chain constant region. In certain embodiments the isolated antibody or antigen binding portion thereof comprises an IgG1 or IgG4 constant region. Where the antibody comprises an IgG4 constant region this may include an S228P mutation.

The invention also provides DNA molecules which encode the isolated antibody or antigen binding portion thereof of the invention. In certain embodiments the sequence of the DNA molecule is selected from any one of the group consisting of SEQ ID NO. 10 to 18, SEQ ID NOS 68 to 115 or a sequence at least 95% identical thereto or a sequence which hybridises thereto under moderate to high stringency conditions.

The invention also provides a method of treating a condition involving NKT cell effector function in a human subject comprising administering to the subject the isolated antibody or antigen binding portion thereof of the invention. Examples of conditions involving NKT cell effector function, such as excessive cytokine production by NKT cells, which may be treated include psoriasis, ulcerative colitis, primary biliary cirrhosis, autoimmune hepatitis, nonalcoholic steatohepatitis, atherosclerosis, ischemia reperfusion injury, asthma and pulmonary inflammation or dysfunction associated with sickle cell disease.

As is described in the following Examples the present inventors have developed potent antibodies which bind to a particular epitope of CD1d. The determination of the nature of this epitope is routine for persons skilled in this area armed with both the antibody and antigen. Methods well known to those skilled in this are which can used to determine the CD1d epitope to which the antibodies 401.11 and 402.8 bind include CD1d alanine scanning mutagenesis, hydrogen/deuterium exchange mapping, X-ray crystallography, nuclear magnetic resonance and photoaffinity labelling.

Alanine-scanning mutagenesis (see for example Ausubel in: Current Protocols in Molecular Biology. Wiley Interscience, ISBN 047 150338, 1987), Chapters 8 and 15; or Cunningham et al. 1989 Science 244 1081-5) introduces single alanine mutations at every residue in the CD1d molecule. The resulting mutant molecules are then tested for their ability to bind the 401.11 and/or 402.8 antibodies. A loss binding means that a particular residue which has been changed to alanine may be involved in the epitope.

In epitope mapping using hydrogen deuterium exchange the hydrogens in CD1d are exchanged with deuterium in solution. The 401.11 and/or 402.8 antibodies are then bound to the CD1d which is then exchanged back in H₂O. In this process the deuterium present in the epitope are protected by the binding of the antibody. A comparison of the exchange patterns with CD1d protected by the antibody binding and unprotected reveals the epitope as the amino acid residues of CD1d retaining deuterium.

In X-ray crystallography CD1d to which the 401.11 and/or 402.8 antibody is bound is crystallised and the crystal examined by X-ray diffraction. This methodology provides clear information as to the region of CD1d to which the antibody is bound. Nuclear magnetic resonance or photoaffinity labelling may also be used as described in de Vos et al. 1992 Science 255 306-12; and Smith et al. 1992 J Mol Biol 224 899-904.

As will be understood by person skilled in this field the epitope recognised by the antibody or antigen binding portion thereof of the invention may comprise a linear series of amino acids or may be a conformational epitope.

In one aspect the invention is directed to antibodies which compete for binding to human CD1d with at least one antibody selected from the group consisting of 401.11 and 402.8.

As used herein “competes” means that the antibody or antigen binding portion thereof reduces the binding of at least one antibody selected from the group consisting of 401.11, 401.11.28, 402.8, 402.8.45, 402.8.53 and 402.8.60 to CD1d in a concentration dependent manner. An example of the way in which this may be assessed is provided in Example 7 set out below. In particular an antibody or antigen binding portion thereof is said to “compete” with at least one antibody selected from the group consisting of 401.11 and 402.8 for binding to CD1d where there is a greater reduction in binding of the at least one antibody selected from the group consisting of 401.11 and 402.8 with the test antibody than with antibody 42 or 51.1 used at the same concentration. (The prior art antibodies 42 and 51.1 are described in Exley et al. 1997 J Exp Med 186, 109-120 and WO03/092615).

As described herein, an antibody or antigen binding portion thereof which “competes for binding to CD1d” demonstrates at least 50% competition in normalized results in a competition ELISA, in which 40 μg/mL of non-biotinylated test antibody is competed with 0.2 μg/mL biotinylated anti-CD1d antibody 402.8 or 401.11 or 401.11.158 bound to 1.0 μg/mL recombinant human CD1d which is immobilized on a solid substrate

In certain embodiments the invention provides an isolated antibody or antigen binding portion thereof binds to CD1d with an EC₅₀ of less than 20 ng/ml as measured using a cell based potency assay. In certain embodiments the isolated antibody or antigen binding portion thereof binds to CD1d with an EC₅₀ of between 0.5 ng/ml to 20 ng/ml. As used herein the EC₅₀ of the antibody or antigen binding portion thereof is to be assessed as in Example 4 as set out below.

As mentioned above the antibodies or antigen binding portions thereof specifically bind CD1d. As used herein the term “specifically” means that the binding to CD1d is via the VH and VL domains of the antibody or antigen binding portion thereof and not a non-specific binding such as may occur via the Fc region.

As described in the following examples the antibodies or antigen binding portions thereof of the invention bind to both human and cynomolgus or rhesus CD1d. This is in contrast to prior art antibodies 42 and 51.1.

The amino acid sequence of human CD1d may be, for example:

(SEQ ID NO: 157)
MGCLLFLLLWALLQAWGSAEVPQRLFPLRCLQISSFANSSWTRTDGLAW
LGELQTHSWSNDSDTVRSLKPWSQGTFSDQQWETLQHIFRVYRSSFTRD
VKEFAKMLRLSYPLELQVSAGCEVHPGNASNNFFHVAFQGKDILSFQGT
SWEPTQEAPLWVNLAIQVLNQDKWTRETVQWLLNGTCPQFVSGLLESGK
SELKKQVKPKAWLSRGPSPGPGRLLLVCHVSGFYPKPVWVKWMRGEQEQ
QGTQPGDILPNADETWYLRATLDVVAGEAAGLSCRVKHSSLEGQDIVLY
WGGSYTSMGLIALAVLACLLFLLIVGFTSRFKRQTSYQGVL

The UniProt accession number for human CD1d is P15813.

In another aspect the invention is directed to antibodies which bind the same epitope of CD1d as that bound by at least one antibody selected from the group consisting of 401.11 and 402.8, (and in some embodiments 40.11.158). As described above the epitope of CD1d bound by a particular antibody can be assessed by a number of methodologies and this can then be compared to the epitope bound by the specified antibody.

In an embodiment the epitope comprises residues 141 to 143 of SEQ ID NO: 116 or residues 87 to 93 and 141 to 143 of SEQ ID NO: 116.

The term antibody as used herein, broadly refers to any immunoglobulin (Ig) molecule comprised of four polypeptide chains, two heavy (H) chains and two light (L) chains, or any functional fragment, mutant, variant, or derivation thereof, which retains the essential epitope binding features of an Ig molecule. Such mutant, variant, or derivative antibody formats are known in the art. Non-limiting embodiments of which are discussed below.

In a full-length antibody, each heavy chain is comprised of a heavy chain variable region (abbreviated herein as HCVR or VH) and a heavy chain constant region. The heavy chain constant region is comprised of three domains, CH1, CH2 and CH3. Each light chain is comprised of a light chain variable region (abbreviated herein as LCVR or VL) and a light chain constant region. The light chain constant region is comprised of one domain, CL. The VH and VL regions can be further subdivided into regions of hypervariability, termed complementarity determining regions (CDR), interspersed with regions that are more conserved, termed framework regions (FR). Each VH and VL is composed of three CDRs and four FRs, arranged from amino-terminus to carboxy-terminus in the following order: FR1, CDR1, FR2, CDR2, FR3, CDR3, FR4. Immunoglobulin molecules can be of any type (e.g., IgG, IgE, IgM, IgD, IgA and IgY), class (e.g., IgG1, IgG2, IgG3, IgG4, IgA1 and IgA2) or subclass.

The term “antigen binding portion” of an antibody, as used herein, refers to one or more fragments of an antibody or protein that retain the ability to specifically bind to an antigen (e.g., CD1d). It has been shown that the antigen-binding function of an antibody can be performed by fragments of a full-length antibody. Such antibody embodiments may also be bispecific, dual specific, or multi-specific formats; specifically binding to two or more different antigens. Examples of binding fragments encompassed within the term “antigen-binding portion” of an antibody include (i) a Fab fragment, a monovalent fragment consisting of the VL, VH, CL and CH1 domains; (ii) a F(ab′)2 fragment, a bivalent fragment comprising two Fab fragments linked by a disulfide bridge at the hinge region; (iii) a Fd fragment consisting of the VH and CH1 domains; (iv) a Fv fragment consisting of the VL and VH domains of a single arm of an antibody, (v) a domain antibody (dAb) (Ward et al., 1989 Nature 341 544-6, Winter et al., PCT publication WO 90/05144 all herein incorporated by reference), which comprises a single variable domain; and (vi) an isolated complementarity determining region (CDR). Furthermore, although the two domains of the Fv fragment, VL and VH, are coded for by separate genes, they can be joined, using recombinant methods, by a synthetic linker that enables them to be made as a single protein chain in which the VL and VH regions pair to form monovalent molecules (known as single chain Fv (scFv); (see e.g., Bird et al. 1988 Science 242 423-6; Huston et al. 1988 Proc Natl Acad Sci USA 85 5879-83). Such single chain antibodies are also intended to be encompassed within the term “antigen-binding portion” of an antibody. Other forms of single chain antibodies, such as diabodies are also encompassed. Diabodies are bivalent, bispecific antibodies in which VH and VL domains are expressed on a single polypeptide chain, but using a linker that is too short to allow for pairing between the two domains on the same chain, thereby forcing the domains to pair with complementary domains of another chain and creating two antigen binding sites (see e.g., Holliger, P., et al., 1993, Proc. Natl. Acad. Sci. USA 90:6444-6448; Poljak, R. J., et al., 1994, Structure 2:1121-1123). Such antibody binding portions are known in the art (Kontermann and Dubel eds., Antibody Engineering 2001 Springer-Verlag. New York. 790 pp., ISBN 3-540-41354-5).

The antibody described herein may be may be a humanized antibody. The term “humanized antibody” shall be understood to refer to a protein comprising a human-like variable region, which includes CDRs from an antibody from a non-human species (e.g., mouse or rat or non-human primate) grafted onto or inserted into FRs from a human antibody (this type of antibody is also referred to a “CDR-grafted antibody”). Humanized antibodies also include proteins in which one or more residues of the human protein are modified by one or more amino acid substitutions and/or one or more FR residues of the human protein are replaced by corresponding non-human residues. Humanized antibodies may also comprise residues which are found in neither the human antibody or in the non-human antibody. Any additional regions of the protein (e.g., Fc region) are generally human. Humanization can be performed using a method known in the art, e.g., U.S. Pat. No. 5,225,539, U.S. Pat. No. 6,054,297, U.S. Pat. No. 7,566,771 or U.S. Pat. No. 5,585,089. The term “humanized antibody” also encompasses a super-humanized protein, e.g., as described in U.S. Pat. No. 7,732,578.

The antibody described herein may be human. The term “human antibody” as used herein refers to proteins having variable and, optionally, constant antibody regions found in humans, e.g. in the human germline or somatic cells or from libraries produced using such regions. The “human” antibodies can include amino acid residues not encoded by human sequences, e.g. mutations introduced by random or site directed mutations in vitro (in particular mutations which involve conservative substitutions or mutations in a small number of residues of the protein, e.g. in 1, 2, 3, 4 or 5 of the residues of the protein). These “human antibodies” do not necessarily need to be generated as a result of an immune response of a human, rather, they can be generated using recombinant means (e.g., screening a phage display library) and/or by a transgenic animal (e.g., a mouse) comprising nucleic acid encoding human antibody constant and/or variable regions and/or using guided selection (e.g., as described in or U.S. Pat. No. 5,565,332). This term also encompasses affinity matured forms of such antibodies. For the purposes of the present disclosure, a human protein will also be considered to include a protein comprising FRs from a human antibody or FRs comprising sequences from a consensus sequence of human FRs and in which one or more of the CDRs are random or semi-random, e.g., as described in U.S. Pat. No. 6,300,064 and/or U.S. Pat. No. 6,248,516.

Amino acid positions assigned to CDRs and FRs may be defined according to Kabat Sequences of Proteins of Immunological Interest, National Institutes of Health, Bethesda, Md., 1987 and 1991 (also referred to herein as “the Kabat numbering system”). In other embodiments, the amino acid positions assigned to CDRs and FRs are defined according to the Enhanced Chothia Numbering Scheme (http://www.bioinfo.org.uk/mdex.html). According to the numbering system of Kabat, VH FRs and CDRs may be positioned as follows: residues 1-30 (FR1), 31-35 (CDR1), 36-49 (FR2), 50-65 (CDR2), 66-94 (FR3), 95-102 (CDR3) and 103-113 (FR4). According to the numbering system of Kabat, VL FRs and CDRs are positioned as follows: residues 1-23 (FR1), 24-34 (CDR1), 35-49 (FR2), 50-56 (CDR2), 57-88 (FR3), 89-97 (CDR3) and 98-107 (FR4). The present disclosure is not limited to FRs and CDRs as defined by the Kabat numbering system, but includes all numbering systems, including the canonical numbering system or of Chothia and Lesk J. Mol. Biol. 196:901-917, 1987; Chothia et al. Nature 342, 877-883, 1989; and/or Al-Lazikani et al., J Mol Biol 273, 927-948, 1997; the numbering system of Honnegher and Plükthun J. Mol. Biol., 309: 657-670, 2001; or the IMGT system discussed in Giudicelli et al., Nucleic Acids Res., 25: 206-211 1997. In one example, the CDRs are defined according to the Kabat numbering system. Optionally, heavy chain CDR2 according to the Kabat numbering system does not comprise the five C-terminal amino acids listed herein or any one or more of those amino acids are substituted with another naturally-occurring amino acid. In an additional, or alternative, option, light chain CDR1 does not comprise the four N-terminal amino acids listed herein or any one or more of those amino acids are substituted with another naturally-occurring amino acid. In this regard, Padlan et al., FASEB J., 9: 133-139, 1995 established that the five C-terminal amino acids of heavy chain CDR2 and/or the four N-terminal amino acids of light chain CDR1 are not generally involved in antigen binding.

The term “antibody construct” as used herein refers to a polypeptide comprising one or more antigen binding portions of the invention linked to a linker polypeptide or an immunoglobulin constant domain. Linker polypeptides comprise two or more amino acid residues joined by peptide bonds and are used to link one or more antigen binding portions. Such linker polypeptides are well known in the art (see e.g. Holliger et al. 1993 Proc Natl Acad Sci USA 90 6444-8).

An immunoglobulin constant domain refers to a heavy or light chain constant domain. Human IgG heavy chain and light chain constant domain amino acid sequences are known in the art and examples are represented below.

Human heavy chain IgG1 constant domain (or derivatives thereof like NCBI
Accession No: P01857)
(SEQ ID NO: 158)
ASTKNPDVFPLAPSSKSTSGGTAALGCLVKDYFPEPVTVSWNSGALTSGVHTFPAVLQSSGLYS
LSSVVTVPSSSLGTQTYICNVNHKPSNTKVDKKVEPKSCDKTHTCPPCPAPELLGGPSVFLFPPK
PKDTLMISRTPEVTCVVVDVSHEDPEVKFNWYVDGVEVHNAKTKPREEQYNSTYRVVSVLTV
LHQDWLNGKEYKCKVSNKALPAPIEKTISKAKGQPREPQVYTLPPSRDELTKNQVSLTCLVKGF
YPSDIAVEWESNGQPENNYKTTPPVLDSDGSFFLYSKLTVDKSRWQQGNVFSCSVMHEALHN
HYTQKSLSLSPGK
Human heavy chain IgG4 constant domain (like NCBI Accession No: P01861)
(SEQ ID NO: 159)
ASTKGPSVFPLAPCSRSTSESTAALGCLVKDYFPEPVTVSWNSGALTSGVHTFPAVLQSSGLYSL
SSVVTVPSSSLGTKTYTCNVDHKPSNTKVDKRVESKYGPPCPSCPAPEFLGGPSVFLFPPKPKDT
LMISRTPEVTCVVVDVSQEDPEVQFNWYVDGVEVHNAKTKPREEQFNSTYRVVSVLTVLHQD
WLNGKEYKCKVSNKGLPSSIEKTISKAKGQPREPQVYTLPPSQEEMTKNQVSLTCLVKGFYPSD
IAVEWESNGQPENNYKTTPPVLDSDGSFFLYSRLTVDKSRWQEGNVFSCSVMHEALHNHYT
QKSLSLSLGK
Human heavy chain IgG4 constant domain incorporating an S228P mutation
(SEQ ID NO: 160)
ASTKGPSVFPLAPCSRSTSESTAALGCLVKDYFPEPVTVSWNSGALTSGVHTFPAVLQSSGLYSL
SSVVTVPSSSLGTKTYTCNVDHKPSNTKVDKRVESKYGPPCPPCPAPEFLGGPSVFLFPPKPKDT
LMISRTPEVTCVVVDVSQEDPEVQFNWYVDGVEVHNAKTKPREEQFNSTYRVVSVLTVLHQD
WLNGKEYKCKVSNKGLPSSIEKTISKAKGQPREPQVYTLPPSQEEMTKNQVSLTCLVKGFYPSD
IAVEWESNGQPENNYKTTPPVLDSDGSFFLYSRLTVDKSRWQEGNVFSCSVMHEALHNHYT
QKSLSLSLGK

Human heavy chain IgG4 constant domain incorporating an S228P mutation and a YTE mutation such as described in U.S. Pat. No. 7,083,784 may also be used.

Human light chain kappa constant domain
(like NCBI Accession No: P01834)
(SEQ ID NO: 161)
TVAAPSVFIFPPSDEQLKSGTASVVCLLNNFYPREAKVQWKVDNALQSG
NSQESVTEQDSKDSTYSLSSTLTLSKADYEKHKVYACEVTHQGLSSPVT
KSFNRGEC
Human light chain lambda constant domain
(like NCBI Accession No: P01842)
(SEQ ID NO: 162)
QPKAAPSVTLFPPSSEELQANKATLVCLISDFYPGAVTVAWKADSSPVK
AGVETTTPSKQSNNKYAASSYLSLTPEQWKSHRSYSCQVTHEGSTVEKT
VAPTECS

As will be appreciated the sequences developed and described in the invention may be modified using methods well known in the art to increase binding, by for example, affinity maturation, or to decrease immunogenicity by removing predicted MHC class II-binding motifs. The therapeutic utility of the sequences developed and described herein can be further enhanced by modulating their functional characteristics, such as antibody-dependent cell-mediated cytotoxicity (ADCC), complement-dependent cytotoxicity (CDC), serum half-life, biodistribution and binding to Fc receptors or the combination of any of these. This modulation can be achieved by protein-engineering, glyco-engineering or chemical methods. Depending on the therapeutic application required, it could be advantageous to either increase or decrease any of these activities.

Numerous methods for affinity maturation of antibodies are known in the art. Many of these are based on the general strategy of generating panels or libraries of variant proteins by mutagenesis followed by selection and/or screening for improved affinity. Mutagenesis is often performed at the DNA level, for example by error prone PCR (Thie H 2009 Methods Mol. Biol. 525:309-22), by gene shuffling (Kolkman and Stemmer 2001 Nat. Biotechnol. May; 19(5):423-8), by use of mutagenic chemicals or irradiation, by use of ‘mutator’ strains with error prone replication machinery (Greener 1996) or by somatic hypermutation approaches that harness natural affinity maturation machinery (Peled, Kuang et al. 2008). Mutagenesis can also be performed at the RNA level, for example by use of Qβ replicase (Kopsidas, Roberts et al. 2006). Library-based methods allowing screening for improved variant proteins can be based on various display technologies such as phage, yeast, ribosome, bacterial or mammalian cells, and are well known in the art (Benhar 2007). Affinity maturation can be achieved by more directed/predictive methods for example by site-directed mutagenesis or gene synthesis guided by findings from 3D protein modeling (see for example Queen, Schneider et al. 1989 or U.S. Pat. No. 6,180,370 or U.S. Pat. No. 5,225,539).

A number of methods for modulating antibody serum half-life and biodistribution are based on modifying the interaction between antibody and the neonatal Fc receptor (FcRn), a receptor with a key role in protecting IgG from catabolism, and maintaining high serum antibody concentration. Dall'Acqua et al., describe substitutions in the Fc region of IgG1 that enhance binding affinity to FcRn, thereby increasing serum half-life (Dall'Acqua, Woods et al., 2002) and further demonstrate enhanced bioavailability and modulation of ADCC activity with triple substitution of M252Y/S254T/T256E (YTE mutation) (Dall'Acqua, Kiener et al., 2006). See also U.S. Pat. Nos. 6,277,375; 6,821,505; and 7,083,784. Hinton et al., have described constant domain amino acid substitutions at positions 250 and 428 that confer increased in vivo half-life (Hinton, Johlfs et al. 2004). (Hinton, Xiong et al. 2006). See also U.S. Pat. No 7,217,797. Petkova et al have described constant domain amino acid substitutions at positions 307, 380 and 434 that confer increased in vivo half-life (Petkova, Akilesh et al. 2006). See also Shields et al (Shields, Namenuk et al. 2001) and WO 2000/42072. Antibody constant regions can also be modified so as to remove effector function. The mutation of the Asparagine (N) at position 297 to a Glutamine (Q) removes the N-linked carbohydrate that mediates binding of the Fc to Fc receptors. Such aglycosylated antibodies do not bind to the human Fc gamma RI and do not activate the complement pathway (Tao and Morrison 1989). Other examples of constant domain amino acid substitutions which modulate binding to Fc receptors and subsequent function mediated by these receptors, including FcRn binding and serum half-life, are described in U.S. Pat. Application Nos 20090142340; 20090068175; and 20090092599.

In molecules of the invention which comprise an Fc region, in some embodiments it may be advantageous to engineer in the substitution L235E to reduce or abolish Fc binding and Fc-related effector function, as described in Lund, Winter et al (1991) J Immunology 147: 2657-2662 and Alegre et al. (1992) J Immunology 148: 3461-3468. The antibody may be an IgG1, and IgG3 or an IgG4.

In molecules of the invention which comprise an Fc region, in some embodiments it may be advantageous to engineer or otherwise select for an Fc in which the C-terminal lysine (K447) is deleted. Preferably this modification improves manufacturability by reducing heterogeneity of expressed molecule.

The glycans linked to antibody molecules are known to influence interactions of antibody with Fc receptors and glycan receptors and thereby influence antibody activity, including serum half-life (Kaneko, Nimmerjahn et al. 2006; Jones, Papac et al. 2007; and Kanda, Yamada et al. 2007). Hence, certain glycoforms that modulate desired antibody activities can confer therapeutic advantage. Methods for generating engineered glycoforms are known in the art and include but are not limited to those described in U.S. Pat. Nos. 6,602,684; 7,326,681; 7,388,081; and WO 2008/006554.

Extension of half-life by addition of polyethylene glycol (PEG) has been widely used to extend the serum half-life of proteins, as reviewed, for example, by Fishburn 2008.

The invention also provides compositions comprising at least one isolated antibody or antigen binding portion thereof of the invention. This composition will typically comprise at least one formulating agent selected from sterile water, sterile buffered water, and/or at least one preservative selected from the group consisting of phenol, m-cresol, p-cresol, o-cresol, chlorocresol, benzyl alcohol, alkylparaben, benzalkonium chloride, benzethonium chloride, sodium dehydroacetate and thimerosal, or mixtures thereof in an aqueous diluent, optionally, wherein the concentration of protein is about 0.1 mg/ml to about 200 mg/ml, further comprising at least one isotonicity agent or at least one physiologically acceptable buffer.

The antibody compositions of the invention can optionally further comprise an effective amount of at least one compound or protein selected from at least one of an anti-infective drug, a cardiovascular (CV) system drug, a central nervous system (CNS) drug, an autonomic nervous system (ANS) drug, a respiratory tract drug, a gastrointestinal (GI) tract drug, a hormonal drug, a drug for fluid or electrolyte balance, a hematologic drug, an antineoplastic, an immunomodulation drug, an ophthalmic, otic or nasal drug, a topical drug, a nutritional drug or the like. Such drugs are well known in the art, including formulations, indications, dosing and administration for each presented herein (see, e.g., Nursing 2001 Handbook of Drugs, 21st edition, Springhouse Corp., Springhouse, Pa., 2001; Health Professional's Drug Guide 2001, ed., Shannon, Wilson, Stang, Prentice-Hall, Inc, Upper Saddle River, N.J.; Pharmcotherapy Handbook, Wells et al., ed., Appleton & Lange, Stamford, Conn., each entirely incorporated herein by reference).

The compositions of the invention can further comprise at least one of any suitable auxiliary, such as, but not limited to, diluent, binder, stabiliser, buffers, salts, lipophilic solvents, preservative, adjuvant or the like. Pharmaceutically acceptable auxiliaries are preferred. Non-limiting examples of, and methods of preparing such sterile solutions are well known in the art, such as, but not limited to, Gennaro, Ed., Remington's Pharmaceutical Sciences, 18 th Edition, Mack Publishing Co. (Easton, Pa.) 1990. Pharmaceutically acceptable carriers can be routinely selected that are suitable for the mode of administration, solubility and/or stability of the antibody composition as well known in the art or as described herein.

Pharmaceutical excipients and additives useful in the present composition include but are not limited to proteins, peptides, amino acids, lipids, and carbohydrates (e.g., sugars, including monosaccharides, di-, tri-, tetra-, and oligosaccharides; derivatized sugars such as alditols, aldonic acids, esterified sugars and the like; and polysaccharides or sugar polymers), which can be present singly or in combination, comprising alone or in combination 1-99.99% by weight or volume. Exemplary protein excipients include serum albumin, such as human serum albumin (HSA), recombinant human albumin (rHA), gelatin, casein, and the like. Representative amino acids which can also function in a buffering capacity include alanine, glycine, arginine, betaine, histidine, glutamic acid, aspartic acid, cysteine, lysine, leucine, isoleucine, valine, methionine, phenylalanine, aspartame, and the like. One preferred amino acid is histidine. A second preferred amino acid is arginine.

Carbohydrate excipients suitable for use in the invention include, for example, monosaccharides, such as fructose, maltose, galactose, glucose, D-mannose, sorbose, and the like; disaccharides, such as lactose, sucrose, trehalose, cellobiose, and the like; polysaccharides, such as raffinose, melezitose, maltodextrins, dextrans, starches, and the like; and alditols, such as mannitol, xylitol, maltitol, lactitol, xylitol sorbitol (glucitol), myoinositol and the like. Preferred carbohydrate excipients for use in the invention are mannitol, trehalose, and raffinose.

Antibody compositions can also include a buffer or a pH adjusting agent; typically, the buffer is a salt prepared from an organic acid or base. Representative buffers include organic acid salts, such as salts of citric acid, ascorbic acid, gluconic acid, carbonic acid, tartaric acid, succinic acid, acetic acid, or phthalic acid; Tris, tromethamine hydrochloride, or phosphate buffers. Preferred buffers for use in the present compositions are organic acid salts, such as citrate.

Additionally, the compositions of the invention can include polymeric excipients/additives, such as polyvinylpyrrolidones, ficolls (a polymeric sugar), dextrates (e.g., cyclodextrins, such as 2-hydroxypropyl-β-cyclodextrin), polyethylene glycols, flavoring agents, antimicrobial agents, sweeteners, antioxidants, antistatic agents, surfactants (e.g., polysorbates such as “TWEEN® 20” and “TWEEN® 80”), lipids (e.g., phospholipids, fatty acids), steroids (e.g., cholesterol), and chelating agents (e.g., EDTA).

These and additional known pharmaceutical excipients and/or additives suitable for use in the antibody compositions according to the invention are known in the art, e.g., as listed in “Remington: The Science & Practice of Pharmacy”, 19 th ed., Williams & Williams, (1995), and in the “Physician's Desk Reference”, 52 nd ed., Medical Economics, Montvale, N.J. (1998), the disclosures of which are entirely incorporated herein by reference. Preferred carrier or excipient materials are carbohydrates (e.g., saccharides and alditols) and buffers (e.g., citrate) or polymeric agents.

The invention also provides a method of treating a condition involving NKT cell effector function comprising administering the antibody or antigen binding portion thereof. As used herein, the term “NKT cell effector function” is intended to encompass NKT cell functions which result from CD1d-restricted glycolipid activation of NKT cells. Such functions include, but are not necessarily limited to, any one or more of tumor necrosis factor alpha (TNF-α), IFN-γ, IL-4, IL-5 or IL-13 release by NKT cells, up-regulation of NKT cell surface FasL expression, the release of a perforin, and the release of granzyme B by NKT cells.

The route of administration may be selected from wide range of routes of administration including parenteral, intramuscular, intravenous, bolus, intraperitoneal, subcutaneous, respiratory, inhalation, topical, nasal, vaginal, rectal, buccal, sublingual, intranasal, subdermal, and transdermal. It is currently believed, however, that the most appropriate route will be parental or inhalation. Additional information regarding inhalation of proteins can be found in Borish L C, et al 1999 Am. J. Respir. Crit. Care Med. 160(6), 1816-1823.

For parenteral administration, the antibody or antibody binding portion thereof can be formulated as a solution, suspension, emulsion or lyophilised powder in association, or separately provided, with a pharmaceutically acceptable parenteral vehicle. Examples of such vehicles are water, saline, Ringer's solution, dextrose solution, and 1-10% human serum albumin. Liposomes and nonaqueous vehicles, such as fixed oils, may also be used. The vehicle or lyophilised powder may contain additives that maintain isotonicity (e.g., sodium chloride, mannitol) and chemical stability (e.g., buffers and preservatives). The formulation is sterilised by known or suitable techniques.

Isolated nucleic acid molecules of the invention can include nucleic acid molecules comprising an open reading frame (ORF), optionally with one or more introns, e.g., but not limited to, at least one specified portion of at least one CDR, as CDR1, CDR2 and/or CDR3 of at least one heavy chain or light chain, respectively; nucleic acid molecules comprising the coding sequence for an antibody or antibody binding portion thereof; and nucleic acid molecules which comprise a nucleotide sequence substantially different from those described above but which, due to the degeneracy of the genetic code, still encode at least one antibody or antibody binding portion thereof as described herein and/or as known in the art. Of course, the genetic code is well known in the art. Thus, it would be routine for one skilled in the art to generate such degenerate nucleic acid variants that code for a specific antibody or antibody binding portion thereof of the invention. See, e.g., Ausubel, et al., supra, and such nucleic acid variants are included in the invention.

As indicated herein, nucleic acid molecules of the invention which comprise a nucleic acid encoding an antibody or antibody binding portion thereof can include, but are not limited to, those encoding the amino acid sequence of an antibody or antibody binding portion thereof, by itself; the coding sequence for the entire antibody or antibody binding portion thereof; the coding sequence for an antibody or antibody binding portion thereof as well as additional sequences, such as the coding sequence of at least one signal leader or fusion peptide, with or without the aforementioned additional coding sequences, such as at least one intron, together with additional, non-coding sequences, including but not limited to, non-coding 5′ and 3′ sequences, such as the transcribed, non-translated sequences that play a role in transcription, mRNA processing, including splicing and polyadenylation signals (for example—ribosome binding and stability of mRNA); an additional coding sequence that codes for additional amino acids, such as those that provide additional functionalities. Thus, the sequence encoding an antibody or antibody binding portion thereof can be fused to a marker sequence, such as a sequence encoding a peptide that facilitates purification of the fused antibody or antibody binding portion thereof.

The invention provides isolated nucleic acids that hybridise under selective hybridisation conditions to a polynucleotide encoding an antibody or antibody binding portion thereof of the invention. Thus, the polynucleotides of this embodiment can be used for isolating, detecting, and/or quantifying nucleic acids comprising such polynucleotides. For example, polynucleotides of the invention can be used to identify, isolate, or amplify partial or full-length clones in a deposited library. In some embodiments, the polynucleotides are genomic or cDNA sequences isolated, or otherwise complementary to, a cDNA from a human or mammalian nucleic acid library.

Preferably, the cDNA library comprises at least 80% full-length sequences, preferably at least 85% or 90% full-length sequences, and more preferably at least 95% full-length sequences. The cDNA libraries can be normalized to increase the representation of rare sequences. Low or moderate stringency hybridisation conditions are typically, but not exclusively, employed with sequences having a reduced sequence identity relative to complementary sequences. Moderate and high stringency conditions can optionally be employed for sequences of greater identity. Low stringency conditions allow selective hybridisation of sequences having about 70% sequence identity and can be employed to identify orthologous or paralogous sequences.

Optionally, polynucleotides of this invention will encode at least a portion of an antibody or antigen binding portion thereof encoded by the polynucleotides described herein. The polynucleotides of this invention embrace nucleic acid sequences that can be employed for selective hybridisation to a polynucleotide encoding an antibody or antigen binding portion thereof of the invention. (See, e.g., Ausubel, supra).

The isolated nucleic acids of the invention can be made using (a) recombinant methods, (b) synthetic techniques, and (c) purification techniques, or combinations thereof, as well-known in the art.

The nucleic acids can conveniently comprise sequences in addition to a polynucleotide of the invention. For example, a multi-cloning site comprising one or more endonuclease restriction sites can be inserted into the nucleic acid to aid in isolation of the polynucleotide. Also, translatable sequences can be inserted to aid in the isolation of the translated polynucleotide of the invention. For example, a hexa-histidine marker sequence provides a convenient means to purify the proteins of the invention. The nucleic acid of the invention, excluding the coding sequence, is optionally a vector, adapter, or linker for cloning and/or expression of a polynucleotide of the invention.

Additional sequences can be added to such cloning and/or expression sequences to optimise their function in cloning and/or expression, to aid in isolation of the polynucleotide, or to improve the introduction of the polynucleotide into a cell. Use of cloning vectors, expression vectors, adapters, and linkers is well known in the art. (See, e.g., Ausubel, supra)

The isolated nucleic acid compositions of this invention, such as RNA, cDNA, genomic DNA, or any combination thereof, can be obtained from biological sources using any number of cloning methodologies known to those of skill in the art. In some embodiments, oligonucleotide probes that selectively hybridise, under stringent conditions, to the polynucleotides of the invention are used to identify the desired sequence in a cDNA or genomic DNA library. The isolation of RNA, and construction of cDNA and genomic libraries, is well known to those of ordinary skill in the art. (See, e.g., Ausubel, supra)

A cDNA or genomic library can be screened using a probe based upon the sequence of a polynucleotide of the invention, such as those disclosed herein. Probes can be used to hybridise with genomic DNA or cDNA sequences to isolate homologous genes in the same or different organisms. Those of skill in the art will appreciate that various degrees of stringency of hybridisation can be employed in the assay; and either the hybridisation or the wash medium can be stringent. As the conditions for hybridisation become more stringent, there must be a greater degree of complementarity between the probe and the target for duplex formation to occur. The degree of stringency can be controlled by one or more of temperature, ionic strength, pH and the presence of a partially denaturing solvent, such as formamide. For example, the stringency of hybridisation is conveniently varied by changing the polarity of the reactant solution through, for example, manipulation of the concentration of formamide within the range of 0% to 50%. The degree of complementarity (sequence identity) required for detectable binding will vary in accordance with the stringency of the hybridisation medium and/or wash medium. The degree of complementarity will optimally be 100%, or 90-100%, or any range or value therein. However, it should be understood that minor sequence variations in the probes and primers can be compensated for by reducing the stringency of the hybridisation and/or wash medium.

Methods of amplification of RNA or DNA are well known in the art and can be used according to the invention without undue experimentation, based on the teaching and guidance presented herein. Known methods of DNA or RNA amplification include, but are not limited to, polymerase chain reaction (PCR) and related amplification processes (see, e.g., U.S. Pat. Nos. 4,683,195, 4,683,202, 4,800,159, 4,965,188, to Mullis, et al.; U.S. Pat. Nos. 4,795,699 and 4,921,794 to Tabor, et al.; U.S. Pat. No. 5,142,033 to Innis; U.S. Pat. No. 5,122,464 to Wilson, et al.; U.S. Pat. No. 5,091,310 to Innis; U.S. Pat. No. 5,066,584 to Gyllensten, et al.; U.S. Pat. No. 4,889,818 to Gelfand, et al.; U.S. Pat. No. 4,994,370 to Silver, et al; U.S. Pat. No. 4,766,067 to Biswas; U.S. Pat. No. 4,656,134 to Ringold) and RNA mediated amplification that uses anti-sense RNA to the target sequence as a template for double-stranded DNA synthesis (U.S. Pat. No. 5,130,238 to Malek, et al., with the tradename NASBA), the entire contents of which references are incorporated herein by reference. (See, e.g., Ausubel, supra)

For instance, PCR technology can be used to amplify the sequences of polynucleotides of the invention and related genes directly from genomic DNA or cDNA libraries. PCR and other in vitro amplification methods can also be useful, for example, to clone nucleic acid sequences that code for proteins to be expressed, to make nucleic acids to use as probes for detecting the presence of the desired mRNA in samples, for nucleic acid sequencing, or for other purposes. Examples of techniques sufficient to direct persons of skill through in vitro amplification methods are found in Ausubel, supra, as well as Mullis, et al., U.S. Pat. No. 4,683,202 (1987); and Innis, et al., PCR Protocols A Guide to Methods and Applications, Eds., Academic Press Inc., San Diego, Calif. (1990). Commercially available kits for genomic PCR amplification are known in the art. See, e.g., ADVANTAGE®-GC Genomic PCR Kit (Clontech). The T4 gene 32 protein (Boehringer Mannheim) can be used to improve yield of long PCR products.

The isolated nucleic acids of the invention can also be prepared by direct chemical synthesis by known methods (see, e.g., Ausubel, et al., supra). Chemical synthesis generally produces a single-stranded oligonucleotide, which can be converted into double-stranded DNA by hybridisation with a complementary sequence, or by polymerisation with a DNA polymerase using the single strand as a template. One of skill in the art will recognise that while chemical synthesis of DNA can be limited to sequences of about 100 or more bases, longer sequences can be obtained by the ligation of shorter sequences. Synthesis of longer sequences by assembly of overlapping oligonucleotides is routine in the art.

The invention further provides recombinant expression cassettes comprising a nucleic acid of the invention. A nucleic acid sequence of the invention, for example, a cDNA or a genomic sequence encoding an antibody or antigen binding portion thereof of the invention, can be used to construct a recombinant expression cassette that can be introduced into at least one desired host cell. A recombinant expression cassette will typically comprise a polynucleotide of the invention operably linked to transcriptional initiation regulatory sequences that will direct the transcription of the polynucleotide in the intended host cell. Both heterologous and non-heterologous (i.e., endogenous) promoters can be employed to direct expression of the nucleic acids of the invention.

In some embodiments, isolated nucleic acids that serve as promoter, enhancer, or other elements can be introduced in the appropriate position (upstream, downstream or in intron) of a non-heterologous form of a polynucleotide of the invention so as to up or down regulate expression of a polynucleotide of the invention. For example, endogenous promoters can be altered in vivo or in vitro by mutation, deletion and/or substitution.

The invention also relates to vectors that include isolated nucleic acid molecules of the invention, host cells that are genetically engineered with the recombinant vectors, and the production of at least one antibody or antigen binding portion thereof by recombinant techniques, as is well known in the art. See, e.g., Ausubel, et al., supra. The polynucleotides can optionally be joined to a vector containing a selectable marker for propagation in a host. Generally, a plasmid vector is introduced in a precipitate, such as a calcium phosphate precipitate, or in a complex with a charged lipid. If the vector is a virus, it can be packaged in vitro using an appropriate packaging cell line and then transduced into host cells.

The DNA insert should be operatively linked to an appropriate promoter. The expression constructs will further contain sites for transcription initiation, termination and, in the transcribed region, a ribosome binding site for translation. The coding portion of the mature transcripts expressed by the constructs will preferably include a translation initiating at the beginning and a termination codon (e.g., UAA, UGA or UAG) appropriately positioned at the end of the mRNA to be translated, with UAA and UAG preferred for mammalian or eukaryotic cell expression.

Expression vectors will preferably but optionally include at least one selectable marker. Such markers include, e.g., but not limited to, methotrexate (MTX), dihydrofolate reductase (DHFR, U.S. Pat. Nos. 4,399,216; 4,634,665; 4,656,134; 4,956,288; 5,149,636; and 5,179,017), ampicillin, neomycin (G418), mycophenolic acid, or glutamine synthetase (GS, U.S. Pat. Nos. 5,122,464; 5,770,359; and 5,827,739) resistance for eukaryotic cell culture, and tetracycline or ampicillin resistance genes for culturing in E. coli and other bacteria or prokaryotics (the above patents are entirely incorporated herein by reference). Appropriate culture mediums and conditions for the above-described host cells are known in the art. Suitable vectors will be readily apparent to the skilled artisan. Introduction of a vector construct into a host cell can be effected by calcium phosphate transfection, DEAE-dextran mediated transfection, cationic lipid-mediated transfection, electroporation, transduction, infection or other known methods. Such methods are described in the art, such as Ausubel, supra, Chapters 1, 9, 13, 15, 16.

At least one antibody, or antigen binding portion thereof of the invention can be expressed in a modified form, such as a fusion protein, and can include not only secretion signals, but also additional heterologous functional regions. For instance, a region of additional amino acids, particularly charged amino acids, can be added to the N-terminus of an antibody, or antigen binding portion thereof to improve stability and persistence in the host cell, during purification, or during subsequent handling and storage. Also, peptide moieties can be added to an antibody, or antigen binding portion thereof of the invention to facilitate purification. Such regions can be removed prior to final preparation of an antibody or at least one fragment thereof. Such methods are described in many standard laboratory manuals, such as Ausubel, supra, Chapters 16, 17 and 18. Those of ordinary skill in the art are knowledgeable in the numerous expression systems available for expression of a nucleic acid encoding a protein of the invention.

Alternatively, nucleic acids of the invention can be expressed in a host cell by turning on (by manipulation) in a host cell that contains endogenous DNA encoding an antibody, or antigen binding portion thereof of the invention. Such methods are well known in the art, e.g., as described in U.S. Pat. Nos. 5,580,734, 5,641,670, 5,733,746, and 5,733,761, entirely incorporated herein by reference.

Illustrative of cell cultures useful for the production of the antibodies, specified portions or variants thereof, are mammalian cells. Mammalian cell systems often will be in the form of monolayers of cells although mammalian cell suspensions or bioreactors can also be used. A number of suitable host cell lines capable of expressing intact glycosylated proteins have been developed in the art, and include the COS-1 (e.g., ATCC CRL 1650), COS-7 (e.g., ATCC CRL-1651), HEK293, BHK21 (e.g., ATCC CRL-10), CHO (e.g., ATCC CRL 1610) and BSC-1 (e.g., ATCC CRL-26) cell lines, COS-7 cells, CHOK1SV cells, hep G2 cells, P3X63Ag8.653, SP2/0-Ag14, 293 cells, HeLa cells and the like, which are readily available from, for example, American Type Culture Collection, Manassas, Va. Preferred host cells include cells of lymphoid origin, such as myeloma and lymphoma cells. Particularly preferred host cells are CHOK1 (ATCC: CRL-9618) or CHOK1SV (e.g. Lonza Biologics).

Expression vectors for these cells can include one or more of the following expression control sequences, such as, but not limited to, an origin of replication; a promoter (e.g., late or early SV40 promoters, the CMV promoter; U.S. Pat. Nos. 5,168,062; 5,385,839), an HSV tk promoter, a pgk (phosphoglycerate kinase) promoter, an EF-1 alpha promoter (U.S. Pat. No. 5,266,491), at least one human immunoglobulin promoter; an enhancer, and/or processing information sites, such as ribosome binding sites, RNA splice sites, polyadenylation sites (e.g., an SV40 large T Ag poly A addition site), and transcriptional terminator sequences. See, e.g., Ausubel et al., supra. Other cells useful for production of nucleic acids or proteins of the invention are known and/or available, for instance, from the American Type Culture Collection Catalogue of Cell Lines and Hybridomas (www.atcc.org) or other known or commercial sources.

When eukaryotic host cells are employed, polyadenlyation or transcription terminator sequences are typically incorporated into the vector. An example of a terminator sequence is the polyadenlyation sequence from the bovine growth hormone gene. Sequences for accurate splicing of the transcript can also be included. An example of a splicing sequence is the VP1 intron from SV40 (Sprague et al. 1983 J Virol 45 773-81). Additionally, gene sequences to control replication in the host cell can be incorporated into the vector, as known in the art.

As will be seen the current specification uses the term “% identical” to describe a number of sequences. As would be understood the term “% identical” means that in a comparison of two sequences over the specified region the two sequences have the specified number of identical residues in the same position. The level of identity may be determined using CLUSTALW with default parameters.

It will also be noted that the sequences are “at least 95% identical” to the comparator sequence. In certain embodiments it is preferred that the sequence is at least 96% or at least 97% or at least 98% or at least 99% identical to the comparator sequence.

The term “moderate stringency” in relation to hybridization conditions as used herein means hybridization and/or washing carried out in 2×SSC buffer, 0.1% (w/v) SDS at a temperature in the range 45° C. to 65° C., or equivalent conditions. The term “high stringency” in relation to hybridization conditions as used herein means a hybridization and/or wash carried out in 0.1×SSC buffer, 0.1% (w/v) SDS, or lower salt concentration, and at a temperature of at least 65° C., or equivalent conditions. Reference herein to a particular level of stringency encompasses equivalent conditions using wash/hybridization solutions other than SSC known to those skilled in the art. For example, methods for calculating the temperature at which the strands of a double stranded nucleic acid will dissociate (also known as melting temperature, or Tm) are known in the art. A temperature that is similar to (e.g., within 5° C. or within 10° C.) or equal to the Tm of a nucleic acid is considered to be high stringency. Medium stringency is to be considered to be within 10° C. to 20° C. or 10° C. to 15° C. of the calculated Tm of the nucleic acid.

Throughout this specification the word “comprise,” or variations such as “comprises” or “comprising”, will be understood to imply the inclusion of a stated element, integer or step, or group of elements, integers or steps, but not the exclusion of any other element, integer or step, or group of elements, integers or steps.

All publications mentioned in this specification are herein incorporated by reference. Any discussion of documents, acts, materials, devices, articles or the like which has been included in the present specification is solely for the purpose of providing a context for the invention. It is not to be taken as an admission that any or all of these matters form part of the prior art base or were common general knowledge in the field relevant to the invention as it existed in Australia or elsewhere before the priority date of each claim of this application.

It must be noted that, as used in the subject specification, the singular forms “a,” “an” and “the” include plural aspects unless the context clearly dictates otherwise.

Having generally described the invention, the same will be more readily understood by reference to the following examples, which are provided by way of illustration and are not intended as limiting.

### EXAMPLES

### General Methods

HEK293/pTT5 Expression System

For all transfections involving the HEK293E/pTT5 expression system, HEK293E cells were cultured in complete cell growth media (1 L of F17 medium (Invitrogen), 9 mL of Pluronic F68 (Invitrogen), 2 mM Glutamine containing 20% (w/v) Tryptone NI (Organotechnie) with Geneticin (50 mg/mL, Invitrogen) at 50 μL/100 mL culture). At the day before transfection, the cells were harvested by centrifugation and resuspended in fresh media without Geneticin. The next day DNA was mixed with a commercial transfection reagent and the DNA transfection mix added to the culture drop-wise. The culture was incubated overnight at 37° C., 5% CO2 and 120 rpm without Geneticin. The next day 12.5 mL of Tryptone and 250 μL of Geneticin were added per 500 mL culture. The culture was incubated at 37° C., 5% CO2 and 120 rpm for seven days, then the supernatants were harvested and purified.

CD1d/β2M Proteins

Human CD1d/β2M was produced in the mammalian HEK293E/pTT5 expression system, using a DNA expression construct coding for the extracellular domain of CD1d with an C-terminally located HIS tag (SEQ ID NO: 19), co-transfected with a DNA expression construct coding for β2M (SEQ ID NO: 20). Culture supernatant containing the secreted CD1d/β2M protein was harvested by centrifugation at 2000 g for 10 mins to remove the cells. The CD1d/β2M protein complex was purified from the supernatant via the His8 affinity tag using a HisTrap™ HP column (GE Healthcare). The eluted protein was buffer-exchanged into PBS using a HiLoad 16/60 Superdex 200 prep grade column (GE Healthcare) and ^{˜}50 kDa fraction was separated by gel filtration on a HiLoad 26/60 Superdex 200 prep grade column (GE Healthcare). Human β2M alone was produced and purified in a similar manner. A similar method of purification was adopted for the purification of other species CD1d (e.g. murine CD1d) and synthetic constructs of CD1d (such as hCD1dmu and mCD1dmu).

To determine the sequence of cynomolgus monkey CD1d, cDNA from monkey spleen was obtained from Biochain. The following primers were used to amplify the CD1d DNA based on rhesus CD1d mRNA (PubMed Accession number: NM_{—}001033114):

(SEQ ID NO: 120)
F1-GTGCCTGCTGTTTCTGCTG
(SEQ ID NO: 121)
R1-TGCCCTGATAGGAAGTTTGC

A PCR was set up that amplified a 1 kb DNA product. This DNA was ligated into pGEM-T Easy (Promega) and sequenced using M13 forward and reverse primers. The sequence was aligned with that of rhesus CD1d (UniProt Accession number: Q4AD67) and found to be identical. The gene sequence was then synthesized, a C-terminal HIS tag added, subcloned into the pTT5 vector and expressed using the HEK-293E/pTT5 system. The protein was purified using Ni chromatography via an introduced HIS tag.

For phage display experiments, recombinant human CD1d/β2M was biotinlyated using an EZ-link Sulfo-NHS-LC-biotin kit (Pierce) at a 3:1 ratio of biotin: CD1d/β2M. Free biotin was removed from the protein preparation by dialysis against PBS using a Slide-A-Lyzer dialysis cassette with a 3.5 kDa molecular weight cut-off. For campaign 2, biotinylated recombinant cynomolgus CD1d/β2M was also prepared as described above.

### Construction of Vectors Expressing Antibodies

VH amino acid chains were expressed with a human constant region (human IgG4 heavy chain CH1, hinge, CH2 and CH3 domains (such as NCBI accession number P01861 with the substitution at S228P). This was achieved by back-translation of amino acid sequences into DNA sequences followed by de novo synthesis and assembly of synthetic oligonucleotides. Following gene synthesis the whole sequence was subcloned into the multiple cloning site of the pTT5 heavy chain vector (Durocher, Y. et al., 2002, Nucleic Acids Res, 30, E9). VL amino acid chains were expressed with a human kappa or lambda light chain constant region (such as NCBI accession number AAI10395 and C6KXN3) by subcloning the sequence into the multiple cloning site of the pTT5 light chain vector.

### Expression and Purification of Antibodies

Heavy and light chain DNA vectors were co-transfected into the HEK293/pTT5 expression system and cultured for seven days. The supernatants derived from these transfections were adjusted to pH 7.4 before being loaded onto a HiTrap Protein A column (5 mL, GE Healthcare). The column was washed with 50 mL of 1×PBS (pH 7.4). Elution was performed using 0.1 M citric acid pH 2.5. The eluted antibody was desalted using Zeba Desalting columns (Pierce) into 1×PBS (pH 7.4). The antibodies were analyzed using SDS-PAGE. The concentration of the antibody was determined using the BCA assay kit (Pierce).

### Example 1

### Generation of Anti-CD1d Antibodies

### Phage Display

FAbs that bind to both human and cynomolgus CD1d/β2M were isolated from a naive phagemid library.

Anti-CD1d/β2M FAbs were isolated from the phage display library over the course of two panning ‘campaigns’ (i.e. discrete phage display experiments with different reagents or panning conditions). The general protocol followed the method outlined by Marks et al. (Marks, J. D. & Bradbury, A., 2004, Methods Mol Biol, 248, 161-76).

Each phage display campaign involved three rounds of panning. For each round, ^{˜}1×10¹³ phage particles were blocked by mixing 1:1 with blocking buffer (5% skim milk in phosphate buffered saline pH 7.4) and incubating for 1 hr at room temperature. The blocked phage library was then pre-depleted for streptavidin binders by incubation for 45 mins with 100 μL of streptavidin-coupled Dynabeads (Invitrogen), which were blocked as described for the library. The beads (and streptavidin binders attached to them) were discarded after the incubation step.

Recombinant CD1d/β2M antigen was prepared for panning by capture onto the surface of streptavidin-coupled Dynabeads (Invitrogen). To achieve this, 10-100 pmols of biotinylated CD1d/β2M was incubated with 100 μL of beads for 45 rains at room temperature. The resulting CD1d/β2M-bead complexes were washed with PBS to remove free CD1d/β2M and then used in the subsequent panning reaction.

Library panning was conducted by mixing the blocked and pre-depleted library with the CD1d/β2M-bead complexes in a 1.5 mL microcentrifuge tube and rotating for 2 hrs at room temperature. Non-specifically bound phage was removed using a series of washes. Each wash involved pulling the bead complexes from the solution onto the tube wall using a magnetic rack, aspirating the supernatant and then re-suspending the beads in fresh wash buffer. This was repeated a number of times with either PBS wash buffer (PBS with 0.5% skim milk) or PBS-T wash buffer (PBS with 0.05% TWEEN-20 (Sigma) and 0.5% skim milk). Phage that remained bound after the washing process were eluted from the CD1d/β2M-bead complexes by incubation with 0.5 mL of 100 mM triethylamine (TEA) (Merck) for 20 mins at room temperature. The eluted ‘output’ phage were neutralized by adding 0.25 mL of 1 M Tris-HCl pH 7.4 (Sigma).

At the end of the first and second rounds of panning, the output phage were added to a 10 mL culture of exponentially growing TG1 E. coli (yeast-tryptone (YT) growth media) and allowed to infect the cells by incubating for 30 mins at 37° C. without shaking, then with shaking at 250 rpm for 30 mins. The phagemids encoding the phage display output were then rescued as phage particles following a standard protocol (Marks, J. D. & Bradbury, A., 2004, Methods Mol Biol, 248, 161-76). At the end of the third panning round TG1 cells were infected with output phage, but the cells were plated on solid YT growth media (supplemented with 2% glucose and 100 μg/mL carbenicillin) at a sufficient dilution to produce discrete E. coli colonies. These colonies were used to inoculate 1 mL liquid cultures to allow expression of FAb fragments for use in screening experiments.

### ELISA-Based Screening of FAbs for CD1d Binding

Each individual E. coil colony was used to express a FAb that was screened for CD1d/β2M binding activity. Colonies were inoculated into 1 mL YT starter cultures (supplemented with 100 μg/mL carbenicillin and 2% glucose) in 96-well deepwell plates (Costar) and incubated overnight at 30° C. with shaking at 650 rpm. These starter cultures were diluted 1:50 into a 1 mL expression culture (YT supplemented with 100 μg/mL carbenicillin only) and grown to an optical density of 0.8-1.0 at 600 nm. FAb expression was induced by adding isopropyl-beta-D-thiogalactopyranoside to a final concentration of 1 mM. Cultures were incubated at 20° C. for 16 hrs.

FAb samples were prepared by harvesting cells by centrifugation (2500 g, 10 mins) and performing a periplasmic extraction. The cell pellet was resuspended in 75 μL of extraction buffer (30 mM Tris-HCl, pH 8.0, 1 mM EDTA, 20% Sucrose) and shaken at 1000 rpm for 10 mins at 4° C. Extract preparation was completed by adding 225 μA of H₂O, shaking at 1000 rpm for 1 hr and clearing the extract by centrifugation at 2500 g for 10 mins. The supernatants were recovered, filtered through Acroprep™ 100 kDa molecular-weight cutoff plates (Pall Corporation) and stored at 4° C. until required for further experiments.

To screen potential human CD1d-binders yielded by phage display by ELISA, human CD1d/β2M (produced in HEK 293E cells and biotinylated as described above) was captured on streptavidin-coated ELISA plates (Pierce) at 1 μg/mL. Plates were then washed and separate FAb samples (prepared as described above) were added to individual wells on the ELISA plates. FAbs were allowed to bind the captured CD1d/β2M for two hours at room temperature and then washed three times with PBS-T and three times with PBS. Bound FAbs were detected using a HRP-conjugated antibody directed against the V5 affinity tag (Sigma) fused to the C-terminus of the FAb heavy chain. The detection antibody was incubated for 1.5 hrs at room temperature. The plates were washed to remove unbound antibody and the assay signal was developed by incubating with 50 μL 3,3′,5,5′-Tetramethylbenzidine (KPL) and quenched with 50 μL 1 M HCl. Assay signals were read at A450 nm using a microplate reader (Bio-Tek). Results were expressed as the raw A450 nm value, where any signal 2-fold greater than the average assay background was defined as ‘positive’.

In later assays, Maxisorp® ELISA plates (Nunc) coated with non-biotinylated human CD1d/β2M, cynomolgus CD1d/β2M or β2M alone were prepared to test the binding of FAb samples. Washing and detection steps were as described above.

SPR-Based Screening of FAbs for CD1d/β2M Binding

SPR screening was conducted using a BIACORE™ 4000 Biosensor (GE Healthcare) in a single concentration analyte pass assay. Approximately 10,000 RU of antiVS antibody (Invitrogen cat#R960CUS) was immobilized on a CM5 Series S Sensor chip, using standard amine coupling chemistry at pH 5.5 on spots 1, 2, 4 & 5 of each of the four flow cells leaving spot 3 unmodified. The running buffer used was HBS-EP+ (GE Healthcare) and all interactions measured at 25° C. and data collection rate set to 10 Hz. Crude periplasmic preparations of V5-tagged FAbs, were diluted two-fold in running buffer before capturing at a flow rate of 10 μL/min for 100 sec (typically around 200 RU of FAb was captured) on spot 1 or 5 of each flow cell. Following a short stabilization period, human or cynomolgus CD1d/β2M was passed over all spots of all four flow cells simultaneously at a flow rate of 30 μL/min for 100 sec. Dissociation of the interaction was measured for 100 sec prior to regeneration back to the anti-V5 antibody using a 30 sec pulse of 100 mM phosphoric acid. Generated sensorgrams were referenced against an adjacent anti-V5 antibody spot for each flow cell, and fitted using a 1:1 Langmuir equation to determine ka, kd and KD.

### Results of the Phage Display Campaigns

Over 4400 clones were screened for binding to human and cynomolgus CD1d/β2M by SPR assays. A total of 51 FAbs were found to have high selectivity for human and cynomolgus CD1d.

### Example 2

### Confirmation of IgG Binding to CD1d

Human-cynomolgus CD1d reactive FAbs were converted to IgG4 format, expressed and purified as described in the General Methods. The purified antibodies were tested for binding to human and cynomolgus CD1d by ELISA and SPR using modified versions of the assays described in Example 1. Briefly, for ELISA assays Maxisorp® ELISA plates (Nunc) were coated with the appropriate antigen at 1 μg/mL. Plates were then washed and purified IgG samples were added to individual wells on the ELISA plates. IgGs were allowed to bind the captured CD1d/β2M for one hour at room temperature and then washed three times with PBS-T and three times with PBS. Bound IgGs were detected using a HRP-conjugated antibody directed against human Fc (Sigma). The detection antibody was incubated for 30 minutes at room temperature. The plates were washed to remove unbound antibody and the assay signal was developed by incubating with 50 μL 3,3′,5,5′-Tetramethylbenzidine (KPL) and quenched with 50 μl 1 M HCl. Assay signals were read at A450 nm using a microplate reader (Bio-Tek). Results were expressed as the raw A450 nm value, where any signal 2-fold greater than the average assay background was defined as ‘positive’.

Purified antibodies were also subjected to full kinetic characterization using a BIACORE™ T100 biosensor (GE Healthcare). Approximately 10,000 RU of anti-human IgG (Invitrogen cat# H10500) was immobilized on a CM5 Series S Sensor chip, using standard amine coupling chemistry in flow cell (FC) 1 and FC2 (or alternatively FC3 and FC4) of the BIACORE™ T100 Biosensor. The running buffer used was HBS-EP-1-(GE Healthcare) and interactions measured at 25° C. Peak purified IgGs were diluted to 10 nM in running buffer, and captured on FC2 (or alternatively FC4) at a flow rate of 10 μL/min in order to capture 50-80 RU of IgG. After an appropriate stabilization period, the target, human or cynomolgus CD1d/β2M was passed over FC1 and FC2 (or alternatively FC3 and FC4) at a flow rate of 60 μL/min at concentrations ranging from 33.3 nM to 0.4 nM (using a three-fold dilution of CD1d/β2M). The contact time for association was 120 sec and dissociation measured for 20 mins for the highest concentration and 240 sec for all other concentrations in the series. The sensorgram data from FC2 was subtracted from FC1 and a buffer only control. The curves were fitted using a 1:1 Langmuir equation to generate the ka, kd and KD values (Table 1).

TABLE 1
ELISA and SPR results for Phage Display Antibodies

| | ELISA | ELISA | SPR human CD1d | | |
|---|---|---|---|---|---|
| IgG | hCD1d | cCD1d | ka (1/Ms) | kd (1/s) | KD (M) |
| 401.1 | + | + | 3.30E+05 | 6.10E−05 | 1.85E−10 |
| 401.3 | + | − | 1.55E+05 | 4.61E−03 | 2.98E−08 |
| 401.9 | + | + | 1.69E+05 | 2.21E−02 | 1.31E−07 |
| 401.11 | + | + | 1.80E+05 | 4.79E−04 | 2.66E−09 |
| 401.12 | + | + | 7.84E+05 | 4.59E−03 | 5.85E−09 |
| 401.14 | + | + | 1.37E+05 | 2.88E−03 | 2.10E−08 |
| 401.22 | + | + | 1.92E+05 | 2.70E−03 | 1.40E−08 |
| 401.24 | + | + | 9.02E+05 | 1.19E−03 | 1.32E−09 |
| 401.26 | + | + | 3.75E+05 | 6.02E−03 | 1.60E−08 |
| 401.28 | + | − | 5.10E+05 | 3.30E−03 | 6.47E−09 |
| 401.30 | + | + | 7.51E+05 | 3.42E−03 | 4.55E−09 |
| 401.33 | + | + | 1.16E+05 | 1.09E−03 | 9.40E−09 |
| 402.1 | + | + | 3.15E+05 | 7.01E−03 | 2.23E−08 |
| 402.2 | + | − | 8.31E+04 | 4.51E−04 | 5.43E−09 |
| 402.4 | + | − | 3.05E+05 | 2.89E−03 | 9.51E−09 |
| 402.5 | + | − | 1.80E+05 | 3.17E−03 | 1.76E−08 |
| 402.6 | + | + | 1.58E+05 | 3.12E−04 | 1.98E−09 |
| 402.7 | + | + | 1.71E+05 | 5.05E−03 | 2.95E−08 |
| 402.8 | + | + | 5.30E+05 | 1.61E−04 | 3.04E−10 |
| 402.9 | + | + | 2.56E+05 | 1.55E−03 | 6.04E−09 |
| 402.11 | + | + | 9.46E+04 | 5.22E−03 | 5.52E−08 |
| 402.12 | + | + | 1.18E+06 | 8.29E−04 | 7.01E−10 |
| 402.15 | + | + | 1.99E+05 | 4.31E−03 | 2.17E−08 |
| 402.16 | + | + | 1.96E+05 | 8.89E−04 | 4.54E−09 |
| 402.17 | + | + | 4.61E+05 | 2.88E−03 | 6.24E−09 |
| 402.18 | + | + | 1.25E+05 | 4.08E−04 | 3.27E−09 |

### Example 3

### Cell-Based CD1d Tetramer Inhibition Potency Assay

### Creation of a Stable NKTCR-Expressing Cell Line

To develop cell-based assays to characterize the biological potency of anti-CD1d antibodies, a stable cell line expressing an NKT cell receptor (NKTCR) was required. The cell line J.RT3-T3.5 (ATCC: TIB-153) was chosen for creation of a stable NKT cell receptor-expressing cell line. J.RT3-T3.5 is derived from the E6-1 clone of Jurkat (ATCC: TIB 152) that lacks the β chain of the T cell antigen receptor. The cells do not express either CD3 or the T cell receptor αβ heterodimer on the surface. J.RT3-T3.5 cells were co-electroporated with two vectors, one containing the α chain of the J3N.5 NKTCR (SEQ ID NO: 21) and the other the β chain of the J3N.5 NKTCR (SEQ ID NO: 22) (Brigl, M., et al., 2006 J Immuno) 176: 3625-34.). This NKT cell receptor is reactive to the glycolipid antigen α-GalCer. These vectors encoding the α and β chains of the NKTCR also express resistance genes to geneticin and blasticidin respectively. Stable incorporation of these vectors was achieved by propagation of these cells in culture medium containing pre-determined concentrations of geneticin and blasticidin.

To derive a clonal line, transfected J.RT3-T3.5 cells were grown to log phase in RPMI 1640 (Gibco) under geneticin and blasticidin selection and limiting diluted at an average of one cell per well in 96-well flat-bottom plates (Corning). To determine stable expression of the transfected NKTCR, viable clones were subcloned into larger volumes in 24-well plates and screened by multi-parameter flow cytometry. Clones were screened for binding to CD1d tetramer (ProImmune), expression of Vα24Jα18, the junctional region of the human iNKTCR, and expression of CD3, a co-receptor for the TCR. Clones with high expression of these markers were selected by high mean fluorescence intensity (MFI) of each of the markers. Stability was confirmed by flow cytometry of the clones after multiple passages into T25 flasks and revival after banking down putative clones at −180° C. in a freezing medium (90% heat-inactivated foetal bovine serum and 10% DMSO). Stable clones were identified and used in cell-based assays to characterize functional potency of the anti-CD1d antibodies.

### CD1d Tetramer Inhibition Potency Assay

A cell-based assay to characterize potency of the anti-CD1d antibodies used the clonal NKT cell line described above, in a flow cytometry-based CD1d tetramer inhibition assay. This assay relied on the ability of the CD1d-tetramer loaded with α-GalCer to bind the NKTCR stably transfected into the J.RT3-T3.5 cells. The potency of anti-CD1d antibodies was determined by the ability of the antibody to inhibit CD1d tetramer binding to the NKTCR present on the stably transfected J.RT3-T3.5 line. The inhibitory antibodies bind to a specific epitope on the CD1d molecule within the tetramer that prevents interaction of the CD1d tetramer with the stably transfected NKTCR on the J.RT3-T3.5 cells. The readout of the assay was a reduction in the mean fluorescence intensity (MFI) of the fluorochrome-conjugated CD1d tetramer. Approximate EC50 values were generated by titration of the anti-CD1d antibody whilst keeping the CD1d tetramer concentration constant. To ensure the reproducibility and reliability of the assay, optimization experiments at different CD1d tetramer concentrations were conducted to determine the best dynamic range. The optimal concentration of the CD1d tetramer was determined to be at a 1:1000 dilution, corresponding to approximately 10 nM.

To perform the assay, antibodies were prepared at decreasing concentrations from 10 μg/mL in 0.1% bovine serum albumin (BSA) in cold 1×PBS at pH 7.4. These antibodies were co-incubated at room temperature in the dark at a 1:1 ratio with the anti-CD1d tetramer at a final concentration of 10 nM for a maximum of 40 minutes. This CD1d-tetramer/anti-CD1d antibody mixture was used to stain NKTCR-stable transfectants of J.RT3-T3.5 cells plated at 1×10⁵ cells per well in 96-well round-bottom plates. Wash steps were done in 0.1% BSA in 1×PBS. Data were acquired by flow cytometry and analyzed using flow cytometry analysis software (FlowJo® Treestar, Inc.).

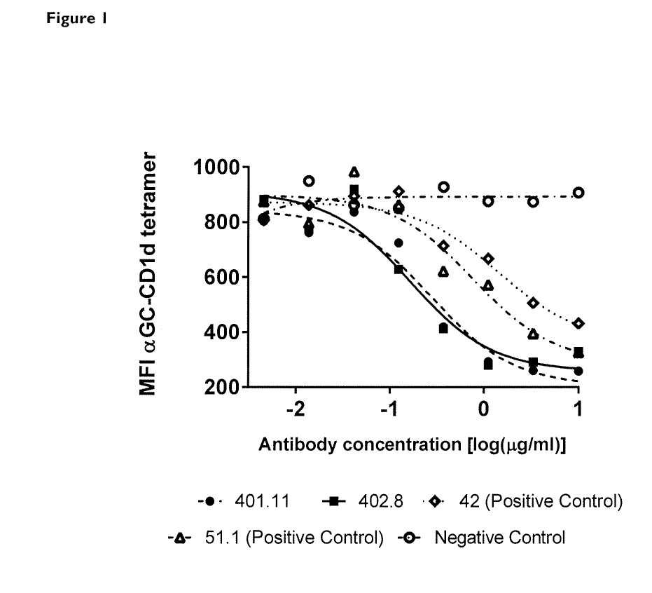

Anti-CD1d antibodies 401.1, 401.9, 401.11, 401.12, 401.14, 401.28, 401.30, 402.1, 402.6, 402.7, 402.8, 402.16, 402.17 and 402.18 were tested in this assay. An irrelevant specificity negative control antibody (human IgG1) was chosen as a negative control. The anti-CD1d antibodies 42 (BD Biosciences) and 51.1 (eBioscience) were chosen as positive controls. Of these antibodies, only 401.11, 401.28, 402.1, 402.6, 402.7, 402.8, 402.16 and 402.18 demonstrated potency in this assay similar or superior to antibody 42 (Table 2). In comparison, the negative control antibody demonstrated negligible inhibition of tetramer binding to the cell line. Representative data from multiple experiments are presented in FIG. 1. This result could not have been predicted by assays that measure the direct binding of antibodies to CD1d. This demonstrates the need to select and screen for antibodies that are capable of functionally inhibiting the CD1d-NKT interaction.

TABLE 2
EC₅₀ values for Tetramer Inhibition Assay

| Antibody Name | EC₅₀ (ng/mL) |
|---|---|
| 401.11 | 283.9 |
| 402.1 | 387.5 |
| 402.6 | 601.6 |
| 402.7 | 791.3 |
| 402.8 | 164.7 |
| 402.16 | 351.6 |
| 402.17 | Negligible Inhibition |
| 402.18 | 88.2 |
| 42 | 1435.0 |
| 51.1 | 775.4 |
| Negative Control | Negligible Inhibition |

### Example 4

### NKT Cell Line IL-2 Release Assay

Anti-CD1d antibodies were further characterized using a cell-line based functional potency assay. The U-937 cell line (ATCC: CRL 1593.2) is a myelomonocytic line that is CD1d-positive. U-937 cells loaded with αGalCer are able to induce the production of IL-2 by the stable NKTCR cell line described in Example 3. Inhibitory anti-CD1d antibodies reduce the release of IL-2 by the NKTCR cell line in response to these αGalCer-loaded U-937 cells. IL-2 levels were measured by standard ELISA technologies (R&D Systems).

To perform the assay, approximately 1.5×10⁵ U-937 cells were loaded with αGalCer at a final concentration of 100 ng/mL in 96-well flat-bottom plates in RPMI 1640 (Gibco). At 60 minutes following the addition of αGalCer, anti-CD1d antibodies were added to the cells at decreasing concentrations starting from 10 μg/mL to the cells. At sixty minutes post antibody loading, 1.5×10⁵ stable NKTCR-transfected J.RT3-T3.5 cells were added to each well. Twenty-four hours after addition of the NKTCR-transfected J.RT3-T3.5 cells, IL-2 levels were tested by ELISA (R&D Systems) using cell-free culture supernatants.

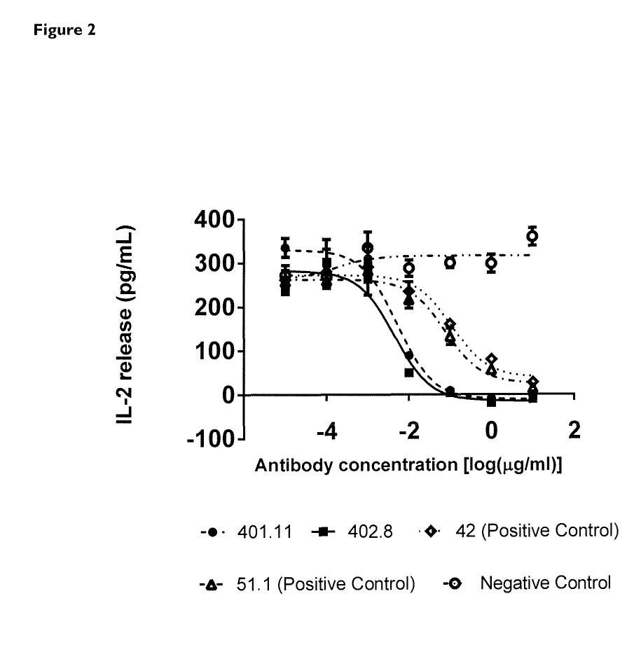

Anti-CD1d antibodies 401.1, 401.9, 401.11, 401.12, 401.14, 401.28, 402.1, 402.6, 402.7, 402.8, 402.16 and 402.18 were tested in this assay. Anti-CD1d antibodies 42 and 51.1 were chosen as positive controls. An irrelevant specificity negative control antibody (human IgG1) was chosen as a negative control. Of these antibodies, only 401.11, 402.1, 402.6, 402.7, 402.8 and 402.16 demonstrated equivalent or stronger inhibition of IL-2 release compared with antibody 42, as determined by EC50 values (Table 3 and representative data in FIG. 2). Additionally, 401.11 and 402.8 demonstrated superior inhibition of IL-2 release compared with the antibody 51.1 (FIG. 2). In comparison, the negative control antibody demonstrated negligible inhibition of IL-2 release. Surprisingly, 401.11 was approximately 20-fold more potent than antibody 42 and approximately 15-fold more potent than 51.1. Similarly, 402.8 was approximately 25-fold more potent than antibody 42 and approximately 17-fold more potent than 51.1 (FIG. 2). Together, these data reveal novel fully human anti-CD1d antibodies with significantly improved biological potency compared with those described in the art.

TABLE 3
EC₅₀ values - NKT Cell Line IL-2 Assay

| Antibody Name | EC₅₀ (ng/mL) |
|---|---|
| 401.1 | Negligible Inhibition |
| 401.9 | 286.0 |
| 401.11 | 5.3 |
| 401.12 | 576.3 |
| 401.14 | Negligible Inhibition |
| 401.28 | 112.4 |
| 401.30 | Negligible Inhibition |
| 402.8 | 4.5 |
| 42 | 110.7 |
| 51.1 | 77.3 |
| (Negative Control) | Negligible Inhibition |

### Example 5

### Testing the Binding of Anti-CD1d Antibodies to Primary PBMCs

Anti-CD1d antibodies were characterized for the ability to bind to CD1d as displayed on primary human somatic cells. Anti-CD1d antibodies 402.8, 401.11.158 and an irrelevant specificity negative control antibody were adjusted to a concentration of 2 mg/mL and conjugated to the fluorochrome Pacific Blue according to the manufacturer's instructions (Invitrogen).

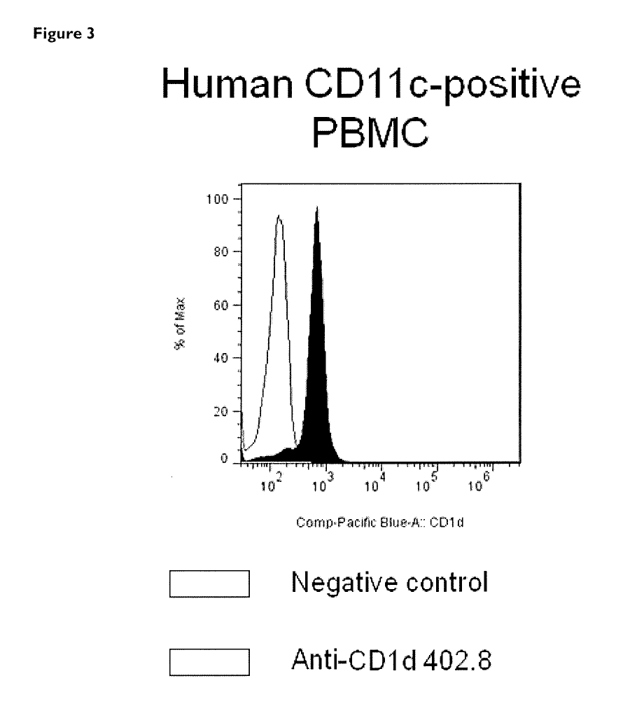

Since it is known that CD1d is expressed on certain human cell populations present in human blood, peripheral blood mononuclear cells (PBMCs) were used to confirm the binding of anti-CD1d antibody 402.8 to primary CD1d+ cells. PBMCs were isolated from buffy coats by density centrifugation over a lymphoprep gradient according to standard protocols (Nycomed). Cells were then washed several times in 1×PBS and stained with anti-CD1d antibody 402.8 (10 μg/mL) or negative control human IgG1 (10 μg/mL), and co-stained with anti-human CD11c (Biolegend). Anti-CD1d antibody 402.8 bound to a distinct CD1d-positive population that was CD11c-positive (FIG. 3). In contrast, the negative control antibody demonstrated negligible binding (FIG. 3). Anti-CD1d antibody 401.11.158 (10 μg/mL) also bound this CD1d-positive population (not shown). These data clearly indicate that anti-CD1d antibodies derived from 402.8 and 401.11, bound to a CD1d+ population in primary human cells.

### Example 6

### Testing the Efficacy of Anti-CD1d Antibodies in Primary NKT Cell-Based Assays

Human NKT cells are capable of eliciting rapid effector function in response to lipid or glycolipid antigens presented in the context of CD1d. This rapid effector function can be demonstrated by release of cytokines such as IFN-γ, IL-4, IL-5, and IL-13. Inhibitory anti-CD1d antibodies can inhibit the function of these NKT cells by binding to CD1d present on cells and preventing the interaction between the NKT cells and their cognate complex of CD1d and glycolipid. Suitable antigen presenting cells may include immortalized myeloid cell lines or primary human dendritic cells. Given the rarity of NKT cells within the peripheral blood of human donors, successful assays require isolation and expansion of such primary NKT cells in the first instance.

### Isolation and Expansion of NKT Cells

PBMCs were isolated from buffy coats over a lymphoprep (Nycomed) gradient. NKT cells were then enriched by standard magnetic-associated cell sorting (MACS) methods (Exley et al., 2010 Curr Protoc Immunol, Chapter 14, Unit 14:11). Briefly, NKT cells were incubated with MACS microbeads against the Vα24-Jα18 iNKT marker (Miltenyi Biotec). Excess microbeads were removed by washing the cell suspension twice in cold PBS. The cell suspension was then passed through the MACS column and the positive fraction containing the enriched NKT cells was retained. Cells from the negative fraction may contain CD1d-positive cells, such as monocytes and dendritic cells, and can be used as feeders to stimulate the enriched NKT cells. The feeder cells are first treated with mitomycin C, an inhibitor of mitosis, for 30 min at 37° C. These cells were then washed several times with tissue culture medium, and then loaded with α-GalCer at a final concentration of 100 ng/mL and co-cultured at a 1:1 ratio with 1×10⁴ NKT cells per well in 96-well round bottom plates. At 16 hours post incubation at 37° C. and 5% CO₂, IL-2 was added to the medium at a final concentration of 10 ng/mL. The cells were left to culture for approximately 14 days. The purity of the NKT cell population was determined by multi-parameter flow cytometry, using fluorochome-conjugated CD1d tetramers (ProImmune), fluorochrome-conjugated anti-Vα24Jα18 (Miltenyi Biotec) and fluorochrome-conjugated anti-CD3 (BD Biosciences). The purity of suitable NKT populations for use in cell-based assays was routinely more than 70% NKT cells by flow cytometry analysis.

### Assay Methodology

All primary cell-based assays were undertaken at 37° C. and 5% CO₂ unless otherwise stated. THP-1 cells were distributed into 96-well flat bottom plates at a concentration of 2×10⁴ cells per well. After ten minutes, α-GalCer was loaded onto the cells at a final concentration of 100 ng/mL. At 45 minutes post addition of α-GalCer, anti-CD1d inhibitory antibodies were added at decreasing concentrations from 10 μg/mL. At 30 minutes post addition of the antibodies, NKT cells were then added at 2×10⁴ cells per well. Cell-free culture supernatants were collected at 24 hours post incubation. ELISA for human cytokines was performed on the culture supernatants: human IFN-γ, IL-4, IL-5 and IL-13 (all R&D Systems).

### Results of the Functional Assays Using Primary NKT Cells

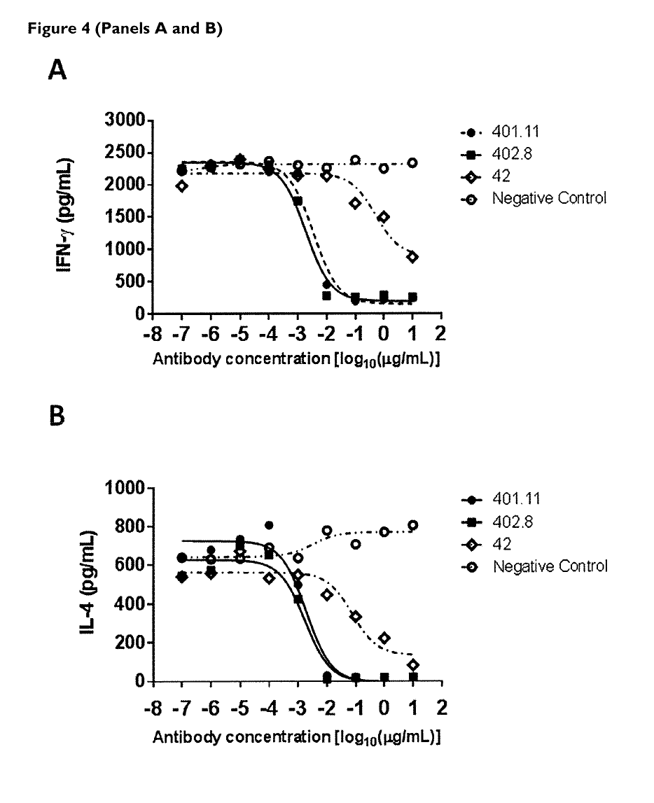

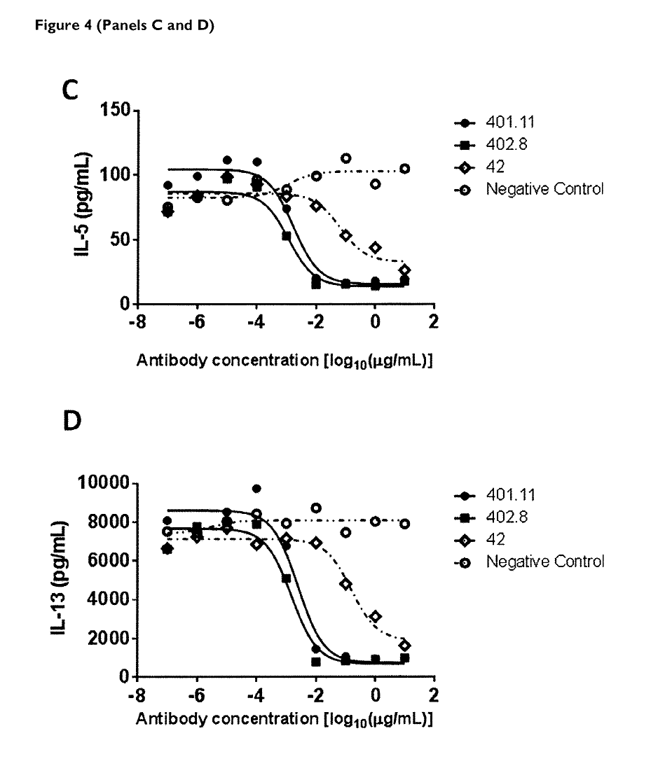

Anti-CD1d antibodies 401.1, 401.9, 401.11, 401.12, 401.14, and 402.8 were tested in this assay. An irrelevant specificity negative control antibody (human IgG1) was used as a negative control. Antibodies 42 and 51.1 were used as positive controls. Similar to the results of the IL-2 cell line assay described in Example 4, only antibodies 401.11 and 402.8 showed strong inhibition of glycolipid-antigen induced cytokine release by primary human NKT cells in the context of cellular CD1d (FIG. 4 and Table 4; see IFN-γ assay EC50 values). By comparison, the negative control antibody demonstrated negligible inhibition. Antibody 42 showed inhibition of cytokine release by NKT cells at high doses (10 μg/mL) but this effect was not sustained at lower concentrations (FIG. 4). Antibody 42 is considered to be a strong neutralizer of NKT cell activity in vitro and is widely published as such (Exley, M. et al., 1997, J. Exp. Med. 186:109420; WO 03/092615). Compared with antibody 42, antibodies 401.11 and 402.8 demonstrated up to 114-fold and up to 180-fold improved potency respectively.

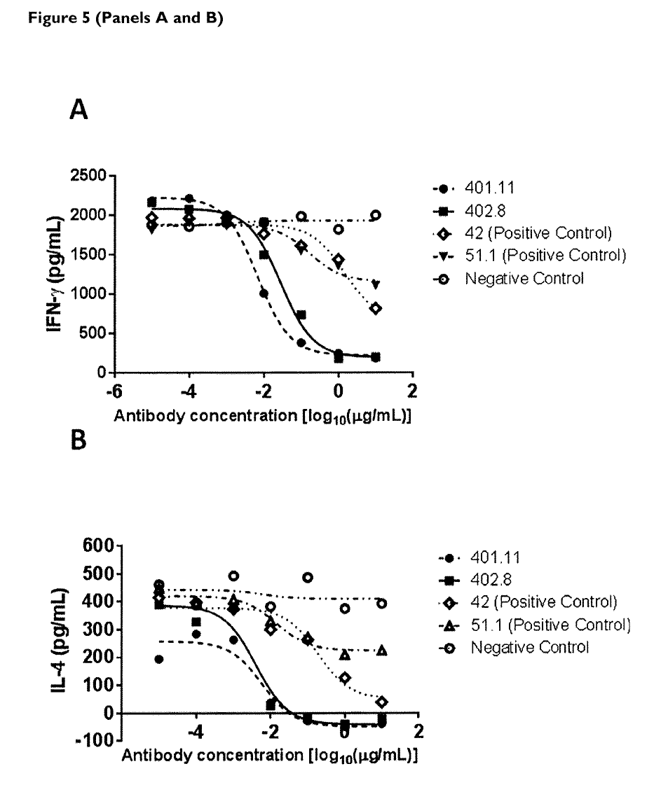

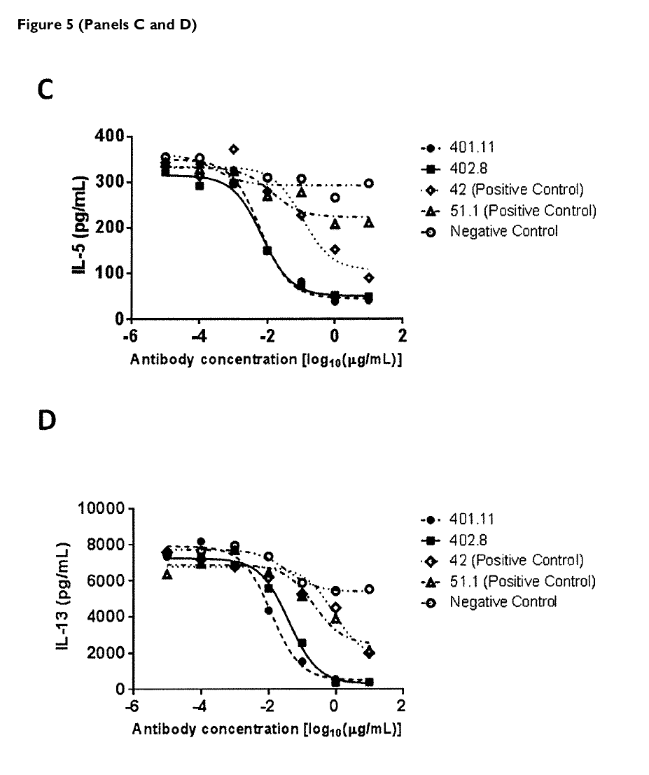

To establish the inhibitory potency of antibodies 401.11 and 402.8 against CD1d present on non-immortalized human cells, a functional assay was developed using primary human monocyte-derived dendritic cells. The dynamic range of the assay may be increased by expanding the proportion of cells that express the CD1d antigen, thereby increasing the level of antigen presentation to CD1d-responsive NKT cells. Monocytes were isolated from PBMC by magnetic activated cell sorting (MACS) isolation of CD14+ cells and culture of these cells in GM-CSF and IL-4 according to standard protocols. Dendritic cells were cultured in 96-well flat bottom plates at 2×10⁴ cells per well and loaded with αGalCer at 100 ng/mL for 1 hr. Inhibitory antibodies were added to the cultures for 1 hr, prior to addition of expanded NKT cells in a 1:1 ratio with the dendritic cells. Twenty-four hours later, cell-free supernatants were assayed for IFN-γ, IL-4, IL-5 and IL-13 release. Anti-CD1d antibodies 401.11 and 402.8 were tested in this assay; an irrelevant specificity human IgG1 was used as a negative control and antibodies 42 (BD Biosciences) and 51.1 (eBioscience) used as positive controls. Only antibodies 401.11 and 402.8 demonstrated strong inhibition of glycolipid-antigen induced cytokine release by primary human NKT cells in this primary cell-based assay (FIG. 5 and Table 5). In comparison, the negative control antibody demonstrated negligible inhibition. Anti-CD1d antibodies 42 and 51.1 showed some inhibition of cytokine release by NKT cells at high doses (10 μg/mL) but this effect was not sustained at lower doses. Compared with antibody 42, antibodies 401.11 and 402.8 demonstrated up to 200-fold and up to 50-fold improved potency respectively (FIG. 5 and Table 5; see IFN-γ assay EC50 values). Compared with antibody 51.1, antibodies 401.11 and 402.8 demonstrated significantly improved potency. This result therefore demonstrates that the anti-CD1d antibodies show potent neutralizing activity in the context of human somatic cells that naturally express the CD1d antigen.

In summary, fully human anti-CD1d antibodies 402.8 and 401.11 were identified and demonstrated highly potent inhibition of NKT cell activity. These antibodies exhibited 100-fold improved potency when compared with the anti-CD1d antibodies 42 and 51.1.

TABLE 4
EC₅₀ values - Primary NKT Cell Line Assays using THP-1 cell line

| | IFN-γ EC₅₀ | IL-4 EC₅₀ | IL-5 EC₅₀ | IL-13 EC₅₀ |
|---|---|---|---|---|
| Antibody Name | (ng/mL) | (ng/mL) | (ng/mL) | (ng/mL) |
| 401.11 | 3.8 | 1.9 | 1.7 | 2.5 |
| 402.8 | 2.4 | 1.7 | 1.2 | 1.5 |
| 42 | 429.1 | 76.1 | 64.7 | 153.8 |
| Negative Control | Negligible | Negligible | Negligible | Negligible |
| | Inhibition | Inhibition | Inhibition | Inhibition |

TABLE 5
EC₅₀ values - Primary NKT Cell Line Assays using primary
CD14+ cells

| | IFN-γ | IL-4 EC₅₀ | IL-5 EC₅₀ | IL-13 EC₅₀ |
|---|---|---|---|---|
| Antibody Name | EC₅₀ (ng/mL) | (ng/mL) | (ng/mL) | (ng/mL) |
| 401.11 | 6.8 | 5.9 | 6.2 | 12.1 |
| 402.8 | 28.6 | 4.0 | 6.9 | 39.9 |
| 51.1 | 168.2 | DNI | DNI | 221.7 |
| 42 | 1388.0 | 185.7 | 108.4 | 844.0 |
| Negative Control | Negligible | Negligible | Negligible | Negligible |
| | Inhibition | Inhibition | Inhibition | Inhibition |

DNI—Did Not Inhibit, where the inhibitory activity of the antibody was typically less than 50% of the maximal response by human NKT cells at 1 μg/mL.

The sequences of the VH and VL domains of antibodies 401.11 and 402.8 are as follows:

| | |
|---|---|
| 401.11 VH | SEQ ID NO 1 |
| 401.11 VL | SEQ ID NO 2 |
| 402.8 VH | SEQ ID NO 3 |
| 402.8 VL | SEQ ID NO 4 |

### Example 7

### 402.8 and 401.11 Share a Common Epitope on CD1d

As shown in the above results, the phage display campaign generated anti-CD1d antibodies 401.11 and 402.8 that showed superior biological potency compared with prior art anti-CD1d antibodies. It was hypothesized that this significantly improved potency was due to recognition of a highly neutralizing epitope that, once bound by the anti-CD1d antibody, prevented the interaction between the CD1d molecule and its cognate receptor, for example the NKT cell receptor present on NKT cells. Blockade of this interaction with CD1d was therefore necessary and sufficient to inhibit downstream biological effects such as activation of NKT cells and the release of pro-inflammatory cytokines. To investigate whether the highly potent anti-CD1d antibodies generated had a different epitope specificity compared with neutralizing anti-CD1d antibodies, a competition binding ELISA was developed.

### Assay Methodology

Anti-CD1d antibody 402.8 was biotinylated using an EZ-link Sulfo-NHS-LC-biotin kit (Pierce) at a 3:1 ratio of biotin: 402.8. Free biotin was removed from the protein preparation by multiple washes with PBS and concentration by centrifugation (3000 rpm) through a centrifugal filter unit with a 30 kDa cutoff (Millipore). Maxisorp® ELISA plates (Nunc) were coated with 0.5 μg/mL human CD1d and allowed to incubate overnight at 4° C. Plates were then washed three times in PBS containing 0.1% Tween® 20, before the plate was blocked in 1% BSA for 1 hr at room temperature. Biotinylated 402.8 was then co-equilibrated for 5 minutes in a 1:1 ratio with non-biotinylated anti-CD1d antibodies (402.8, 401.11, 42 and 51.1). These antibodies were added to the plates for 1 hour at room temperature in decreasing concentrations from 50 μg/mL (i.e. a maximum of 500-fold excess compared with 0.1 μg/mL biotinylated 402.8). Plates were then washed three times in PBS containing 0.1% Tween® 20. Streptavidin horseradish peroxidase conjugate (BD Biosciences) was added to the plates for 1 hour at room temperature in the dark. The plates were washed to remove unbound streptavidin-horseradish peroxidase. The assay signal was developed by incubating with 50 μL 3,3′,5,5′-Tetramethylbenzidine (KPL) and quenched with 50 μL 1 M HCl. Assay signals were read at A450 nm using a microplate reader (FluoStar Galaxy). Results were expressed as the raw A450 nm value and converted to degree of competition (percentage) values by subtracting the readings corresponding with zero percent inhibition from raw data.

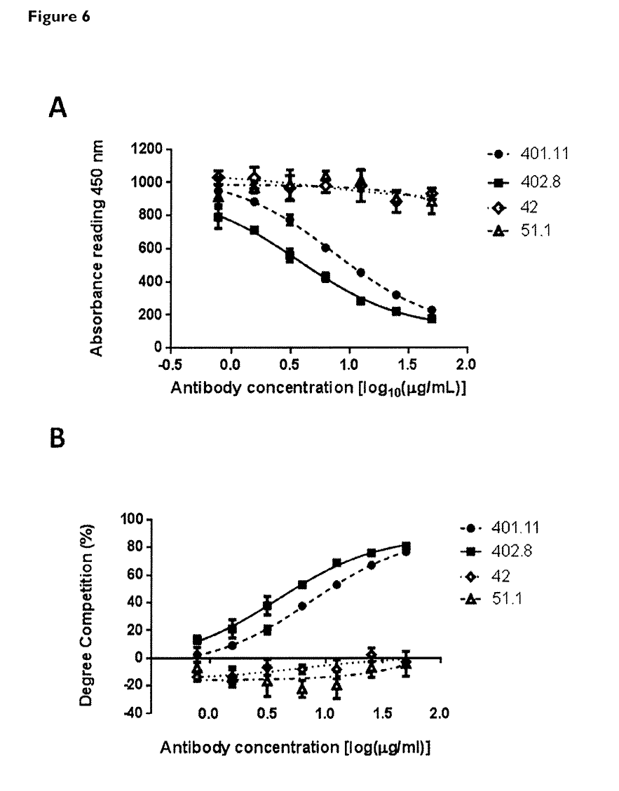

Using the above described method it was demonstrated that 401.11 and 402.8 compete with each other for binding to human CD1d, as shown by absorbance values at 450 nm (FIG. 6A) and degree of competition with 402.8 (FIG. 6B) and therefore share an overlapping or common epitope. In contrast, 402.8 does not share an overlapping or common epitope with either 42 or 51.1. Taken together, these data demonstrate that the highly potent anti-CD1d antibodies 401.11 and 402.8 bind to a similar high affinity neutralizing epitope that is not shared by anti-CD1d antibodies 42 and 51.1.

### Example 8

### Cross-Reactivity with Cynomolgus Macaque CD1d

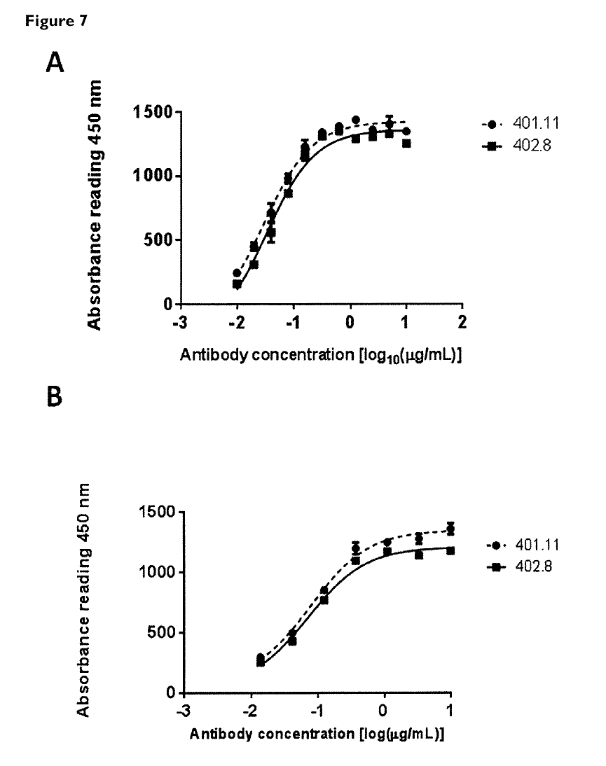

Anti-CD1d antibodies 401.11 and 402.8 were tested for binding to cynomolgus CD1d by ELISA using modified versions of the assays described in Example 1. Maxisorp® ELISA plates (Nunc) were coated with 1 μg/mL human or cynomolgus CD1d and allowed to incubate overnight at 4° C. Plates were then washed three times in PBS containing 0.1% Tween20, before the plate was blocked in 1% BSA for 1 hr at room temperature. Plates were then washed three times in PBS containing 0.1% Tween20. Anti-CD1d antibodies were then added at decreasing concentrations from 10 μg/mL. Plates were then washed three times in PBS containing 0.1% Tween® 20. Detection of the bound antibody was enabled using an HRP-conjugated Fc-specific antibody (Sigma). Plates were then washed three times in PBS containing 0.1% Tween® 20 to remove unbound horseradish peroxidase-conjugated anti-Fc. The assay signal was developed by incubating with 50 μL 3,3′,5,5′-Tetramethylbenzidine (KPL) and quenched with 50 μL 1 M HCl. Assay signals were read at A450 nm using a microplate reader (FluoStar Galaxy). The results indicate that antibodies 401.11 and 402.8, which bind human CD1d (FIG. 7A), are also cross-reactive with cynomolgus CD1d (FIG. 7B). Cross-reactivity with non-human primate CD1d is desirable to allow for testing in non-human primate models of human diseases.

### Example 9

### Cell-Based Cross-Reactivity with Cynomolgus CD1d

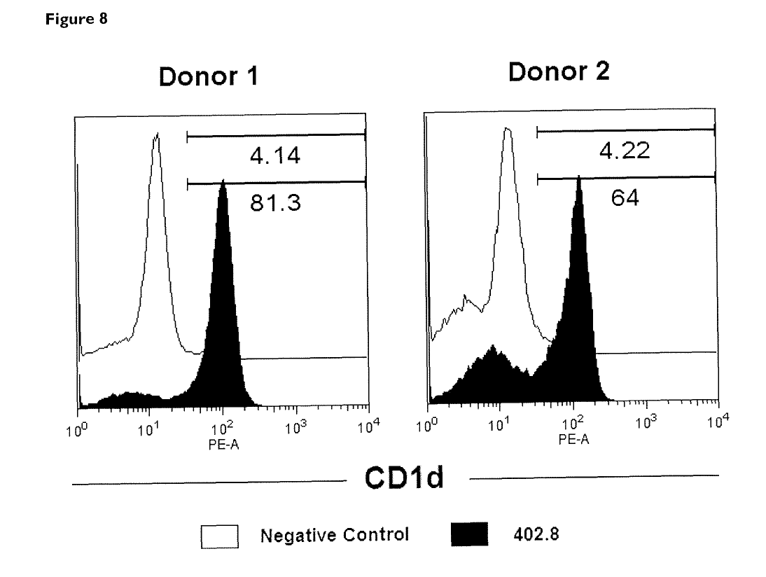

To demonstrate cross-reactivity with non-human primate CD1d in a cell-based format, human and cynomolgus macaque PBMCs were stained using cross-reactive anti-CD1d antibody 402.8. This antibody was biotinylated in a 3:1 fold ratio of biotin to IgG as described in Example 8. A negative control antibody (human IgG1) was biotinylated in a similar manner. Detection of the bound biotinylated anti-CD1d antibody was achieved by incubating the cells with phycoerythrin-conjugated streptavidin. Anti-CD1d antibody bound CD1d-positive primary monocyte-derived DCs in both human (FIG. 3) and cynomolgus macaque species (FIG. 8). These results indicate 402.8 displays human and cynomolgus macaque cross-reactivity in a cell-based context, which is important for testing in non-human primate models of human diseases.

### Example 10

### Cell-Based Functional Inhibition of Cynomolgus CD1d-Mediated Primary NKT Function

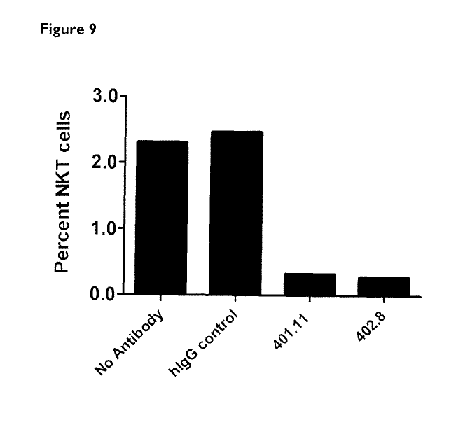

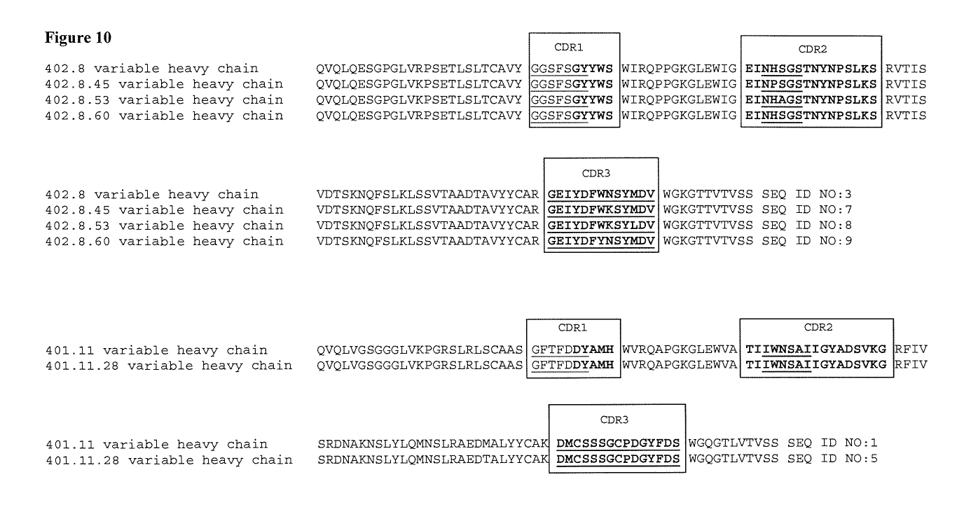

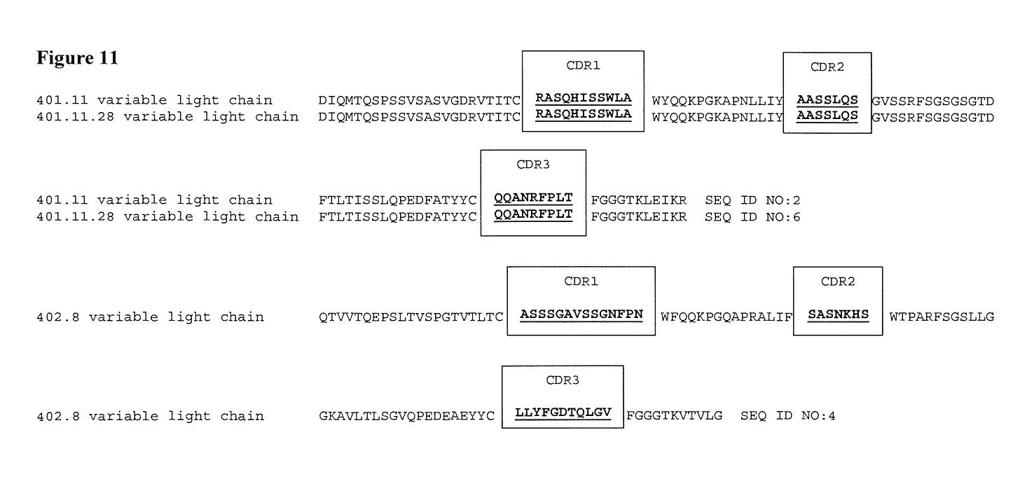

For cell based potency assays, cynomolgus PBMC were loaded on day 0 with αGalCer (100 ng/mL) and with or without anti-CD1d antibodies. The cultures were prepared in 24-well plates in a humidified incubator at 37° C., 5% CO₂. At day 7, IL-2 (10 U/mL) was added on day 7 and the cultures left to incubate at 37° C., 5% CO₂ for a further 96 hours. The final readout was enumeration of NKT cells using anti-CD3 and anti-T cell receptor Va24 antibodies (BD Biosciences). In the absence of anti-CD1d antibody, or with addition of isotype control antibody, NKT cells expanded in the presence of αGalCer by approximately 10-fold. Anti-CD1d antibodies 401.11 and 402.8 potently blocked the αGalCer-mediated expansion of CD1d-restricted cynomolgus NKT cells compared with treatment of cultures with no antibody or human IgG negative control antibody (FIG. 9).

### Example 11

### Optimized Variants of 401.11 and 402.8

The 401.11 and 402.8 antibodies can be further optimized through alterations to the antibody's sequence with the aim of yielding a positive effect on the antibody's biophysical properties whilst having negligible or positive impact on their potency. Firstly, alterations that enhance the expression level of the antibody with concomitantly increased production levels may be desirable. Secondly, removal of potentially undesirable sequence features, such as solvent-exposed cysteine residues or N-linked glycosylation sites through amino acid substitution may reduce potential product heterogeneity, which may further enhance these antibodies. Thirdly, substitution of amino acid residues with the potential to impact the stability of the antibody through oxidation or isomerization during purification or storage may be replaced with amino acids that do not undergo such transitions (Wang et al. 2007 Journal of Pharmaceutical Sciences 96:1-26), which may further improve these antibodies. Lastly, rare or non-germ-line 401.11 and 402.8 amino acid residues which may potentially contribute to immunogenicity may be substituted with other amino acids with the aim of lowering predicted immunogenicity, which may further improve these antibodies. The following describes the implementation of these optimization strategies on the antibodies 401.11 and 402.8.

### Enhancing Antibody 401.11

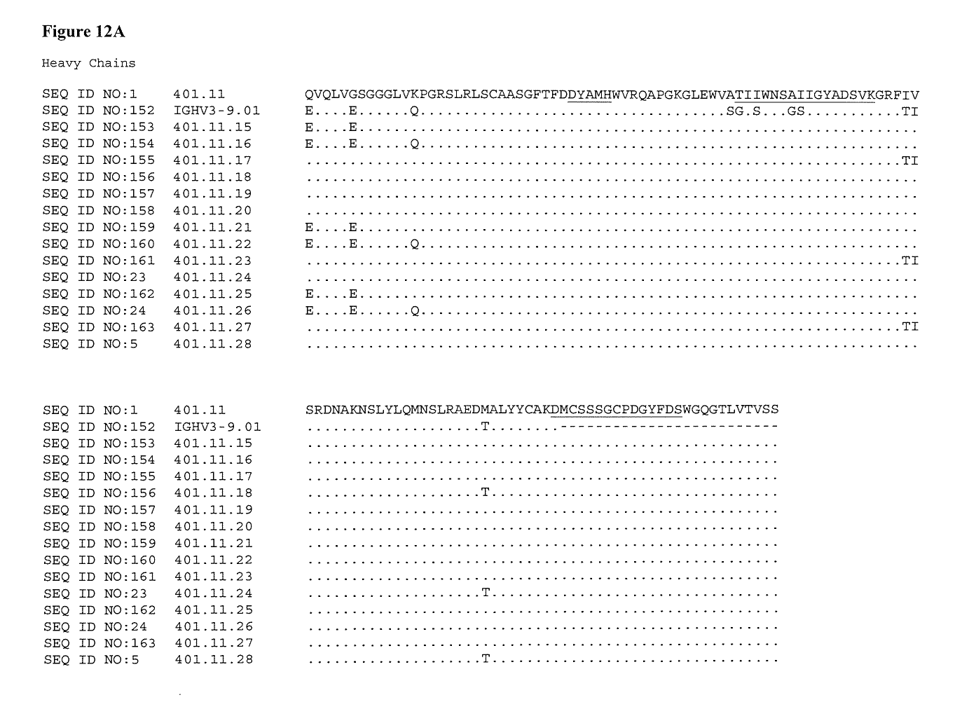

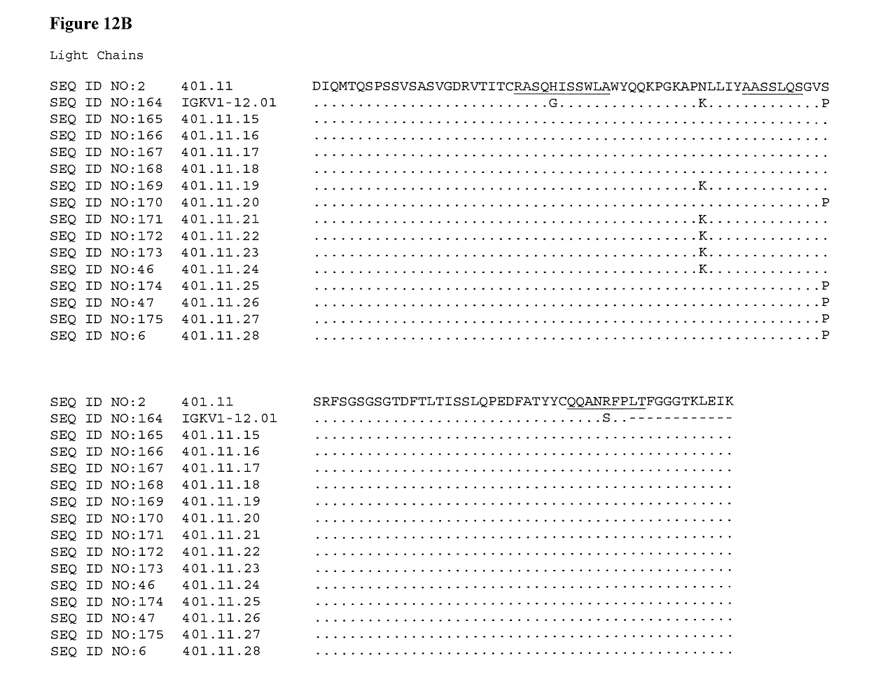

The variable heavy and light chain sequences of 401.11 were compared to corresponding human germline sequences via MegAlign (DNAstar). The most homologous germline heavy chain variable region—IGHV3-9*01—differed from 401.11 by seven framework amino acids. IGKV1-12*01 shared the highest sequence homology with the 401.11 light chain, differing by two framework amino acids (FIGS. 12A and 12B). This information was used to generate a panel of 401.11 variants containing framework residues substituted with the corresponding germline framework residue (FIGS. 12A and 12B).

Antibodies 401.11 and 401.11.15 through 401.11.28 (FIGS. 12A and 12B), were produced by co-transfections of the heavy- and light chains into HEK-293E cells. SPR (BICACORE™) was used to measure the relative expression level of each antibody and its corresponding binding to human CD1d as measured by the equilibrium dissociation constant (KD). The resulting data is presented in Table 6.

TABLE 6

| | Expression | | | |
|---|---|---|---|---|
| | Level | | | |
| | (Protein A | | NKT IFN-γ | NKT IFN-γ |
| | capture) | KD | EC₅₀ (ng/mL) | EC₅₀ (ng/mL) |
| IgG | SPR | (pM) | (THP-1) | (moDC) |
| 401.11 | DNE | N/A | 3.754 | 0.857 |
| 401.11.14 | 144 | 791 | N/D | N/D |
| 401.11.15 | 44 | 507 | N/D | N/D |
| 401.11.16 | 150 | DNB | N/D | N/D |
| 401.11.17 | DNE | N/A | N/D | N/D |
| 401.11.18 | 185 | 9970 | N/D | N/D |
| 401.11.19 | 385 | 667 | N/D | N/D |
| 401.11.20 | 109 | 977 | N/D | N/D |
| 401.11.21 | 534 | 951 | N/D | N/D |
| 401.11.22 | 553 | 754 | N/D | N/D |
| 401.11.23 | 227 | 1980 | N/D | N/D |
| 401.11.24 | 359 | 644 | 4.263 | 0.360 |
| 401.11.25 | 292 | 932 | N/D | N/D |
| 401.11.26 | 358 | 855 | 3.245 | 1.304 |
| 401.11.27 | 23 | DNB | N/D | N/D |
| 401.11.28 | 176 | 722 | 1.417 | 0.294 |

Note:
DNE—Did not express;
DNB—Did not bind;
N/D—not determined;
THP-1 - THP-1 cells used as antigen presenting cells;
moDC - primary monocyte-derived dendritic cells used as antigen presenting cells. Data are representative of 3 independent experiments.

Of the fifteen antibodies tested in this experiment, thirteen had measurable levels of antibody in the supernatant of the transfected HEK-293E cells. Ten of these thirteen antibodies bound to CD1d with an equilibrium dissociation constant (KD) of less than 1 nM (Table 6).

Antibodies 401.11, 401.11.24, 401.11.26 and 401.11.28 were generated and tested for functional inhibition of CD1d mediated NKT cell cytokine release using a cell-based potency assay (Table 6). Antibodies 401.11.24, 401.11.26 and 401.11.28 showed similar or improved potency compared to 401.11 when either THP-1 cells or primary CD14+ dendritic cells were used as CD1d-positive antigen presenting cells (APCs).

Positions 97 through (100B) of CDR3 of 401.11 heavy chain consists of the sequence CSSSGC. To determine the role of the cysteines present in the CDR3 each cysteine was substituted with one of nine amino acids representing the different classes of side chains of amino acids (Rajpal et al PNAS 2005 102: 8466-8471). Following transfection into HEK293E cells no antibody expression was detectable for any of these variants, resulting in no detectable binding to CD1d as measured by SPR. Substitution of both cysteines to serine residues (401.11.164) resulted in an antibody that expressed, but which bound to human CD1d with a lower affinity compared to 401.11 suggesting that the cysteines of CDR3 of the heavy chain of 401.11 are desirable for antibody expression and high affinity binding to CD1d (Table 7).

TABLE 7

| | Substitution | | |
|---|---|---|---|
| | from 401.11 | | |
| | in variable | | |
| IgG | heavy chain | Light Chain | KD (M) |
| 401.11 WT | N/A | 401.11 | 9.97E−10 |
| Mock | N/A | 401.11 | N/D |
| 401.11.51 | C97S | 401.11 | N/D |
| 401.11.52 | C97T | 401.11 | N/D |
| 401.11.53 | C97G | 401.11 | N/D |
| 401.11.54 | C97L | 401.11 | N/D |
| 401.11.55 | C97V | 401.11 | N/D |
| 401.11.56 | C97K | 401.11 | N/D |
| 401.11.57 | C97Y | 401.11 | N/D |
| 401.11.58 | C97R | 401.11 | N/D |
| 401.11.59 | C97H | 401.11 | N/D |
| 401.11.92 | C(100B)S | 401.11 | N/D |
| 401.11.93 | C(100B)T | 401.11 | N/D |
| 401.11.94 | C(100B)G | 401.11 | N/D |
| 401.11.95 | C(100B)L | 401.11 | N/D |
| 401.11.96 | C(100B)V | 401.11 | N/D |
| 401.11.97 | C(100B)K | 401.11 | N/D |
| 401.11.98 | C(100B)Y | 401.11 | N/D |
| 401.11.99 | C(100B)R | 401.11 | N/D |
| 401.11.100 | C(100B)H | 401.11 | N/D |
| 401.11.164 | C97S & | N45K | 6.56E−08 |
| | C(100B)S | | |

N/D—not determined

The heavy chain CDR3 sequence of 401.11 was targeted for further variation to attempt to improve the expression levels and affinity of 401.11. For this analysis, each amino acid in CDR3 of the heavy chain of 401.11 was substituted with one of nine amino acids representing the different classes of side chains of amino acids. The expression levels of each resulting antibody and its binding to CD1d are given in Table 8 and Table 9.

TABLE 8

| | Substitution | Expression | |
|---|---|---|---|
| IgG | VH | Level | KD (M) |
| 401.11 | N/A | 36 | <0.1E−10* |
| Mock | N/A | −8 | DNB |
| 401.11.36 | D95K | DNE | N/A |
| 401.11.37 | D95R | DNE | N/A |
| 401.11.38 | D95S | 105 | DNB |
| 401.11.39 | D95G | 34 | N/A |
| 401.11.40 | D95L | DNE | N/A |
| 401.11.41 | D95Y | DNE | N/A |
| 401.11.42 | D95F | DNE | N/A |
| 401.11.43 | D95Q | DNE | N/A |
| 401.11.44 | M96K | 85 | 4.31E−10 |
| 401.11.45 | M96R | 63 | 2.49E−10 |
| 401.11.46 | M96F | DNE | 3.03E−10 |
| 401.11.47 | M96Y | DNE | N/A |
| 401.11.48 | M96Q | DNE | N/A |
| 401.11.49 | M96S | 47 | 4.01E−10 |
| 401.11.50 | M96G | 29 | 1.87E−10 |
| 401.11.60 | S98D | DNE | N/A |
| 401.11.61 | S98T | 57.85 | 2.42E−18 |
| 401.11.62 | S98W | DNE | N/A |
| 401.11.63 | S98L | 23.76 | 1.89E−10 |
| 401.11.64 | S98V | 84.5 | 2.30E−10 |
| 401.11.65 | S98K | 38.36 | 1.67E−09 |
| 401.11.66 | S98R | 75.87 | 1.56E−10 |
| 401.11.67 | S98Y | DNE | 4.85E−11 |
| 401.11.68 | S98G | 84.81 | 1.17E−09 |
| 401.11.69 | S99D | 57.92 | 1.44E−10 |
| 401.11.70 | S99T | 20.14 | 5.49E−10 |
| 401.11.71 | S99W | 63.04 | <0.1E−10* |
| 401.11.72 | S99L | 11.43 | 6.49E−10 |
| 401.11.73 | S99V | 61.56 | 5.27E−10 |
| 401.11.74 | S99K | 56.91 | 9.33E−11 |
| 401.11.75 | S99R | 14.06 | DNB |
| 401.11.76 | S99Y | DNE | N/A |
| 401.11.77 | S99G | 20.18 | 2.72E−10 |
| 401.11.78 | S99D | DNE | N/A |
| 401.11.79 | S100T | 16.81 | DNB |
| 401.11.80 | S100W | 29.03 | 2.34E−09 |
| 401.11.81 | S100L | 24.65 | 5.52E−10 |
| 401.11.82 | S100V | 20.35 | DNB |
| 401.11.83 | S100K | 45.84 | 1.55E−09 |
| 401.11.84 | S100R | 51.11 | 1.12E−09 |
| 401.11.85 | S100Y | 9.87 | 8.23E−08 |

Residue numbering according to Kabat .<0.1E−10* indicates the KD of the construct was below the limit of detection.
DNE—Did not express (below 10RU);
DNB—Did not bind;
N/A—Not Applicable

TABLE 9

| | Substitution | Expression | |
|---|---|---|---|
| IgG | VH | Level | KD (M) |
| 401.11 | N/A | 103 | 1.75E−10 |
| Mock | N/A | 3 | DNB |
| 401.11.86 | S100G | 337 | 1.39E−10 |
| 401.11.87 | G(100A)S | 24 | 7.13E−09 |
| 401.11.88 | G(100A)A | 103 | 1.89E−09 |
| 401.11.89 | G(100A)D | 40 | 5.30E−09 |
| 401.11.90 | G(100A)K | 21 | <0.1E−10* |
| 401.11.101 | P(100C)S | 95 | <0.1E−10* |
| 401.11.102 | P(100C)T | 130 | 4.57E−10 |
| 401.11.103 | P(100C)G | 115 | <0.1E−10* |
| 401.11.104 | P(100C)L | 44 | 4.87E−10 |
| 401.11.105 | P(100C)F | 34 | 1.52E−03 |
| 401.11.106 | P(100C)K | 64 | 1.93 |
| 401.11.107 | P(100C)Y | 38 | 9.96E−10 |
| 401.11.108 | P(100C)R | 49 | <0.1E−10* |
| 401.11.109 | P(100C)W | 16 | DNB |
| 401.11.110 | D(100D)K | 59 | 2.47E−10 |
| 401.11.111 | D(100D)R | 19 | <0.1E−10* |
| 401.11.112 | D(100D)S | 95 | 2.43E−09 |
| 401.11.113 | D(100D)G | 59 | 6.79E−10 |
| 401.11.114 | D(100D)L | 121 | 1509.04 |
| 401.11.115 | D(100D)Y | 86 | 6.67E−10 |
| 401.11.116 | D(100D)F | 96 | 3.77E−08 |
| 401.11.117 | D(100D)Q | 164 | 1.33E−09 |
| 401.11.118 | G(100E)S | 68 | <0.1E−10* |
| 401.11.119 | G(100E)A | 24 | 8.77E−10 |
| 401.11.120 | G(100E)V | DNE | N/A |
| 401.11.121 | G(100E)D | DNE | N/A |
| 401.11.122 | G(100E)K | 30 | 1.30E−10 |
| 401.11.123 | G(100E)R | 22 | 2.39E−10 |
| 401.11.124 | Y(100F)W | DNE | 1.38E−09 |
| 401.11.125 | Y(100F)F | 33 | 5.35E−10 |
| 401.11.126 | Y(100F)S | 38 | 3.13E−11 |
| 401.11.127 | Y(100F)Q | 26 | 2.70E−09 |
| 401.11.128 | Y(100F)E | DNE | N/A |
| 401.11.129 | Y(100F)R | DNE | N/A |
| 401.11.130 | Y(100F)K | DNE | N/A |
| 401.11.131 | S102D | 38 | 2.00E−10 |
| 401.11.132 | S102T | 55 | 5.69E−10 |
| 401.11.133 | S102W | 53 | 1.67E−10 |
| 401.11.134 | S102L | 73 | 5.32E−10 |
| 401.11.135 | S102V | 85 | 4.21E−10 |
| 401.11.136 | S102K | 22 | <0.1E−10* |
| 401.11.137 | S102R | 49 | 9.38E−10 |
| 401.11.138 | S102Y | 48 | 1.71E−10 |
| 401.11.139 | S102G | 44 | 5.94E−10 |

Residue numbering according to Kabat .<0.1E−10* indicates the KD of the construct was below the limit of detection of the machine.
DNE—Did not express (below 10RU);
DNB—Did not bind;
N/A—Not Applicable

Antibody 401.11.86 expressed at over 3 times the level of 401.11 and had a higher affinity for CD1d compared to 401.11. This antibody was purified and tested for functional inhibition of CD1d mediated NKT cell cytokine release using a cell-based potency assay (Table 10). The assay used primary human monocyte-derived dendritic cells or THP-1 cells as a source of CD1d-positive antigen presenting cells and αGalCer-expanded NKT cells. The protocol was as described in Example 6.

TABLE 10

| | | | NKT | NKT |
|---|---|---|---|---|
| | | | IL-4 EC₅₀ | IL-4 EC₅₀ |
| | NKT IFN-γ EC₅₀ | NKT IFN-γ EC₅₀ | (ng/mL) | (ng/mL) |
| Antibody | (ng/mL) (THP-1) | (ng/mL) (moDC) | (THP-1) | (moDC) |
| 401.11 | 3.754 | 0.857 | 2.885 | 6.862 |
| 401.11.86 | 1.289 | 0.359 | 0.509 | 1.74 |

Note:
THP-1 - THP-1 cells used as antigen presenting cells;
moDC - primary monocyte-derived dendritic cells used as antigen presenting cells. Data are representative of 5 independent experiments.

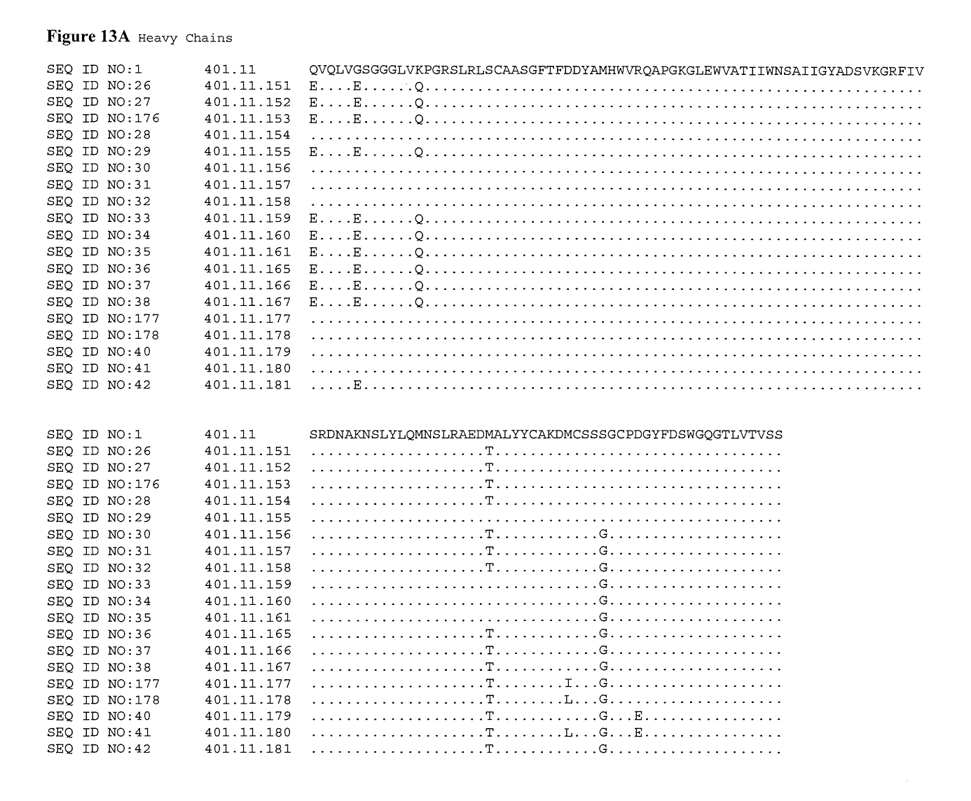

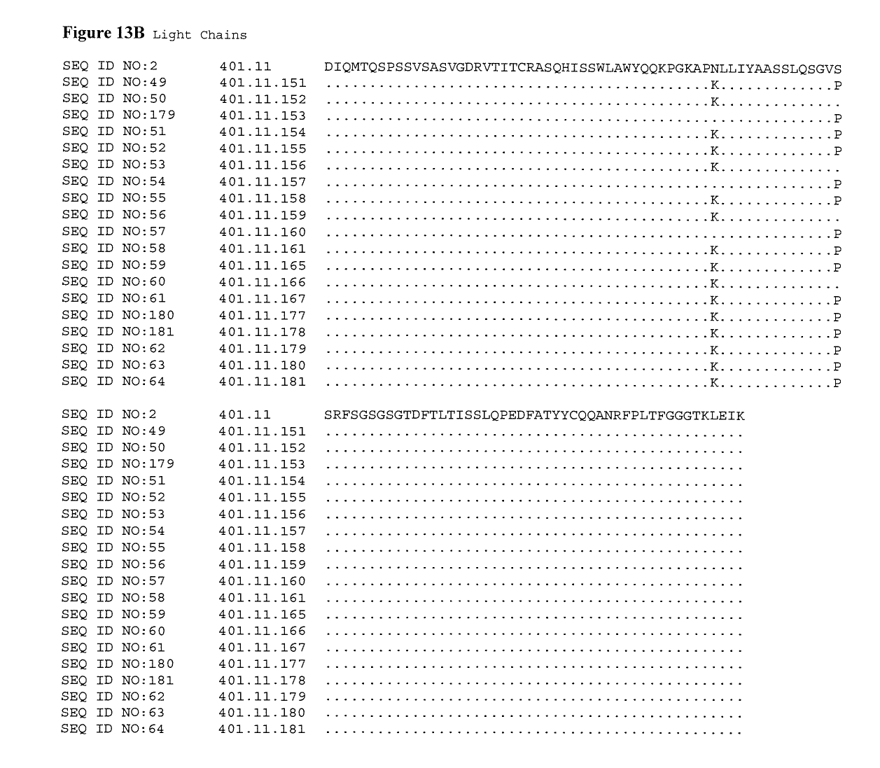

401.11.86, which differed from 401.11 by a Serine to Glycine substitution at position 100, was more potent compared to 401.11. Antibodies were then generated that contained substitutions identified from the most potent antibodies described above (FIGS. 13A and 13B).

Amino acid analysis of the variable heavy chain sequence of 401.11 identified several amino acids that may potentially undergo oxidation or isomerization. Particular emphasis was placed on amino acids present in the CDR sequences of the antibody as any changes to these amino acids may, over time, impact the binding profile of the antibody. In the variable heavy chain, M96 was identified as a potential oxidation site, and D(100D) was identified as a potential isomerization site. Semi conservative or conservative amino acid substitutions were used in attempts to remove these potentially problematic amino acid residues (FIGS. 13A and 13B). The influence of these substitutions on the binding affinity of the resulting antibodies is shown in Table 11.

TABLE 11

| | Affinity K_{D} |
|---|---|
| Antibody | (pM) SPR |
| 401.11 | 752 |
| 401.11.151 | 1750 |
| 401.11.152 | 2830 |
| 401.11.154 | 1770 |
| 401.11.155 | 2420 |
| 401.11.156 | 223 |
| 401.11.157 | 173 |
| 401.11.158 | 189 |
| 401.11.159 | 30 |
| 401.11.160 | 109 |
| 401.11.161 | 255 |
| 401.11.165 | 168 |
| 401.11.166 | 173 |
| 401.11.167 | 170 |
| 401.11.177 | 507 |
| 401.11.178 | 468 |
| 401.11.179 | 274 |
| 401.11.180 | 312 |
| 401.11.181 | 427 |

Many of the antibodies tested had improved affinity compared to the original antibody 401.11. These antibodies were then tested in a cell-based potency assay which measures functional inhibition of CD1d mediated NKT cell cytokine release. The protocol was as described in Example 6. Of the 19 antibodies tested, 14 antibodies consistently demonstrated similar or improved potency compared with the 401.11 antibody. These antibody variants had significantly improved potency compared with anti-CD1d antibodies 42 and 51.1, both of which showed some inhibitory activity at the highest concentration of 10 μg/mL but failed to show inhibition at lower antibody concentrations. The EC₅₀ values from representative experiments are presented in Tables 12-16 below.

TABLE 12

| | NKT | NKT IL-4 | NKT IL-13 | NKT IL-13 |
|---|---|---|---|---|
| | IL-4 EC₅₀ | EC₅₀ | EC₅₀ | EC₅₀ |
| | (ng/mL) | (ng/mL) | (ng/mL) | (ng/mL) |
| Antibody | (moDC) | (moDC) | (moDC) | (moDC) |
| 401.11 | 7.633 | 6.545 | 22.75 | 19.55 |
| 401.11.151 | 12.89 | N/D | 57.49 | N/D |
| 401.11.152 | 10.2 | N/D | 78.49 | N/D |
| 401.11.154 | 4.159 | N/D | 17.73 | N/D |
| 401.11.155 | 27.64 | N/D | 74.72 | N/D |
| 401.11.156 | 1.948 | N/D | 5.217 | N/D |
| 401.11.157 | N/D | 0.799 | N/D | 3.871 |
| 401.11.158 | N/D | 0.58 | N/D | 3.844 |
| 401.11.159 | N/D | 1.694 | N/D | 10.47 |
| 401.11.160 | N/D | 1.564 | N/D | 6.519 |
| 401.11.161 | N/D | 5.244 | N/D | 8.381 |
| Negative Control | DNI | DNI | DNI | DNI |

N/D—not determined;
Negative Control - An IgG1 of irrelevant specificity;
moDC - primary monocyte-derived dendritic cells used as antigen presenting cells;
DNI—Did Not Inhibit, where the inhibitory activity of the antibody was typically less than 50% of the maximal response by human NKT cells at 1 μg/mL.

TABLE 13

| | NKT | | | | |
|---|---|---|---|---|---|
| | IFN-γ | NKT IL-4 | NKT IL-5 | NKT IL-13 | NKT TNF |
| | EC₅₀ | EC₅₀ | EC₅₀ | EC₅₀ | EC₅₀ |
| | (ng/mL) | (ng/mL) | (ng/mL) | (ng/mL) | (ng/mL) |
| Antibody | (moDC) | (moDC) | (moDC) | (moDC) | (moDC) |
| 401.11 | 39.38 | 17.33 | 47.48 | 83.08 | 16.91 |
| 401.11.156 | 7.763 | 2.716 | 5.856 | 9.528 | 4.507 |
| 401.11.157 | 9.112 | 3.437 | 8.492 | 20.6 | 5.589 |
| 401.11.158 | 8.165 | 2.187 | 6.13 | 9.823 | 4.3 |
| 42 | >1000 | 136.9 | 286.5 | DNI | DNI |
| 51.1 | >1000 | DNI | DNI | DNI | DNI |

Note:
moDC—primary monocyte-derived dendritic cells used as antigen presenting cells;
DNI—Did Not Inhibit, where the inhibitory activity of the antibody was typically less than 50% of the maximal response by human NKT cells at 1 μg/mL.

TABLE 14

| | NKT | NKT | | | |
|---|---|---|---|---|---|
| | IFN-γ | IL-4 EC₅₀ | NKT IL-5 | NKT IL-13 | NKT TNF |
| | EC₅₀ | (ng/mL) | EC₅₀ | EC₅₀ | EC₅₀ |
| | (ng/mL) | (THP- | (ng/mL) | (ng/mL) | (ng/mL) |
| Antibody | (THP-1) | 1) | (THP-1) | (THP-1) | (THP-1) |
| 401.11 | 55.16 | 2.977 | 6.961 | 27.25 | 9.179 |
| 401.11.156 | 5.811 | 1.993 | 4.446 | 6.909 | 3.846 |
| 401.11.157 | 5.449 | 1.807 | 4.476 | 6.98 | 3.545 |
| 401.11.158 | 6.404 | 2.662 | 4.502 | 7.776 | 4.141 |
| 401.11.165 | 6.153 | 1.112 | 3.730 | 5.762 | 3.043 |
| 401.11.166 | 6.983 | 2.745 | 5.379 | 7.253 | 4.022 |
| 401.11.167 | 6.510 | 2.889 | 4.979 | 7.465 | 4.000 |
| Isotype | DNI | DNI | DNI | DNI | DNI |
| Control | | | | | |

Note:
Isotype Control - An irrelevant specificity IgG4;
DNI—Did Not Inhibit, where the inhibitory activity of the antibody was typically less than 50% of the maximal response by human NKT cells at 1 μg/mL;
THP-1-THP-1 cells used as antigen presenting cells;
moDC - primary monocyte-derived dendritic cells used as antigen presenting cells.

TABLE 15

| | | | | NKT |
|---|---|---|---|---|
| | | | NKT IL-5 | IL-13 |
| | | | EC50 | EC50 |
| | NKT IFN-γ EC50 | NKT IL-4 EC50 | (ng/mL) | (ng/mL) |
| Antibody | (ng/mL) (moDC) | (ng/mL) (moDC) | (moDC) | (moDC) |
| 401.11 | 12.44 | 1.98 | 2.251 | 2.361 |
| 401.11.158 | 2.797 | 0.4322 | 0.7043 | 0.5981 |
| 401.11.177 | 7.556 | 0.7306 | 1.398 | 1.271 |
| 401.11.178 | 15.06 | 0.9685 | 1.381 | 1.764 |
| 401.11.179 | 30.2 | 0.7301 | 2.097 | 2.431 |
| 401.11.180 | 49.55 | 1.312 | 3.353 | 4.092 |
| 42 | DNI | DNI | DNI | DNI |
| 51.1 | DNI | DNI | DNI | DNI |
| Isotype | DNI | DNI | DNI | DNI |
| Control | | | | |

Note:
Isotype Control - An irrelevant specificity IgG4;
DNI—Did Not Inhibit, where the inhibitory activity of the antibody was typically less than 50% of the maximal response by human NKT cells at 1 μg/mL;
THP-1 - THP-1 cells used as antigen presenting cells;
moDC - primary monocyte-derived dendritic cells used as antigen presenting cells.

TABLE 16

| | NKT | | | | NKT |
|---|---|---|---|---|---|
| | IFN-γ | | NKT IL-4 | NKT IL-4 | IL-13 |
| | EC₅₀ | NKT IFN-γ | EC₅₀ | EC₅₀ | EC₅₀ |
| | (ng/mL) | EC₅₀ (ng/mL) | (ng/mL) | (ng/mL) | (ng/mL) |
| Antibody | (moDC) | (THP-1) | (moDC) | (THP-1) | (THP-1) |
| 401.11 | 23.5 | 2.335 | 12.83 | 1.36 | 3.286 |
| 401.11.158 | 3.004 | 2.602 | 1.160 | 2.203 | 4.354 |
| 401.11.181 | 2.634 | 3.255 | 1.102 | 3.195 | 5.04 |
| 42 | DNI | DNI | DNI | DNI | DNI |
| 51.1 | DNI | DNI | DNI | DNI | DNI |
| Isotype | DNI | DNI | DNI | DNI | DNI |
| Control | | | | | |

Note:
Isotype Control - An irrelevant specificity IgG4;
DNI—Did Not Inhibit, where the inhibitory activity of the antibody was typically less than 50% of the maximal response by human NKT cells at 1 μg/mL;
THP-1 - THP-1 cells used as antigen presenting cells;
moDC - primary monocyte-derived dendritic cells used as antigen presenting cells.

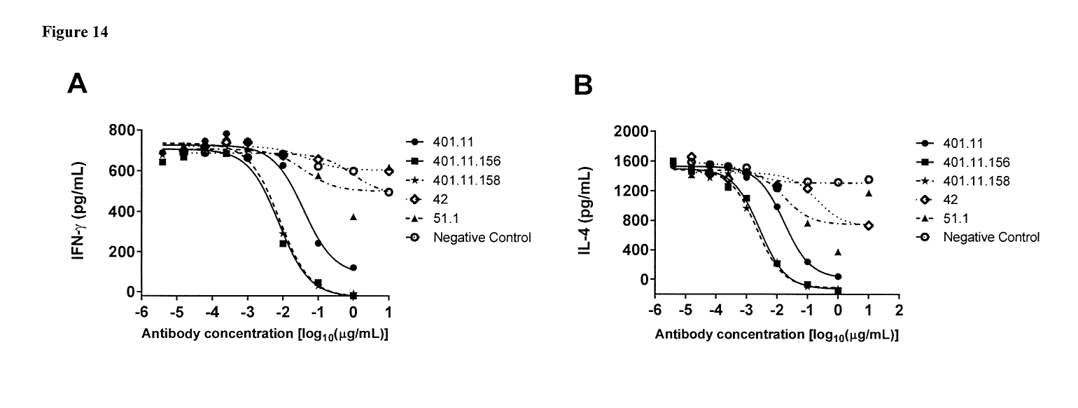

In primary NKT cell-based potency assays using CD14+ monocyte derived dendritic cells as antigen-presenting cells, antibodies derived from 401.11 showed improved inhibitory activity compared with the parental antibody 401.11. This is clearly shown by a left-shift in the inhibition curve, where antibodies derived from 401.11 required lower concentrations to achieve the same inhibition of NKT-cell mediated cytokine release (FIG. 14). For example, antibodies 401.11.156 and 401.11.158, titrated from 1 μg/ml, showed approximately 5.1-fold improvement and 4.8-fold improvement respectively compared with 401.11, titrated from 1 μg/mL (FIG. 14 and Table 13, see IFN-γ EC50 values). In several experiments, anti-CD1d antibodies 42 and 51.1 showed minimal inhibition such that a true EC50 value could not be calculated. In experiments where the EC50 values could be determined, antibodies derived from 401.11, titrated from 1 μg/mL showed significantly improved potency compared with anti-CD1d antibodies 42 and 51.1. These antibodies showed inhibitory activity at the highest concentration of 10 μg/mL, but failed to show inhibition at lower antibody concentrations (FIG. 14 and Table 13); see IFN-γ EC₅₀ values).

In summary, fully human optimized anti-CD1d antibodies derived from 401.11 were identified and demonstrated highly potent inhibition of NKT cell activity in the context of primary human cells that naturally express the CD1d antigen. These antibodies showed significantly improved potency compared with the anti-CD1d antibodies 42 and 51.1.

### Optimization of the 402.8 Antibody

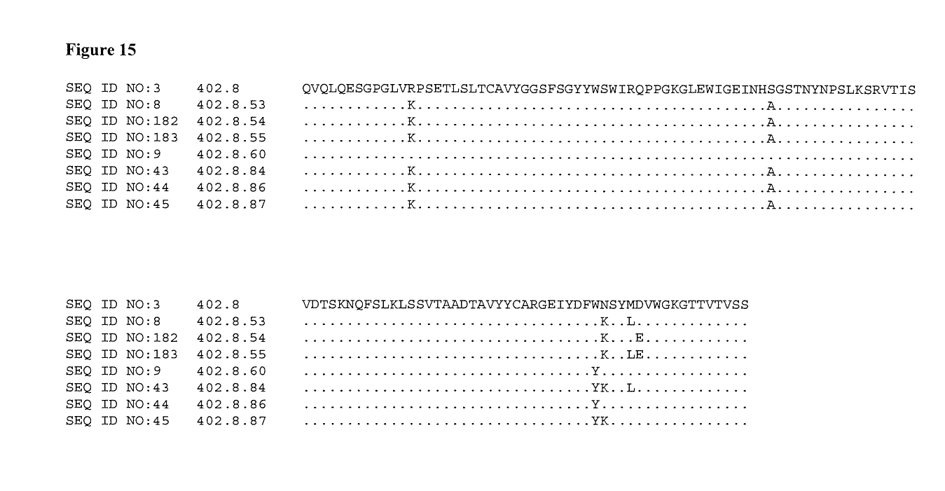

Amino acid analysis of the variable heavy and light chain sequence of 402.8 identified several amino acids that could potentially undergo oxidation, isomerization or deamindation present in the heavy chain (Wang et al. 2007 Journal of Pharmaceutical Sciences 96:1-26). These include a potential deamidation site at N(100B), a potential isomerization site at D101 and potential oxidation sites at W(100A) and M(100E) in the heavy chain. A potential N-linked glycosylation site was identified at N52 in the heavy chain. To remove the potential deamidation, oxidation and isomerization sites the amino acid substitutions were made: W(100A)Y, N(100B)K, M(100E)L, D(101)E. The potential N-linked glycoyslation site was removed by introducing an N54A which disrupts the N-linked glycoyslation motif NX(S/T), where X is any amino acid except proline. Antibodies were made with combinations of these amino acid substitutions in the variable heavy chain as shown in FIG. 15. Each antibody heavy chain was co-transfected with the 402.8 light chain (SEQ ID No: 4) into HEK-293E cells, purified by Protein A chromatography and the affinity of each antibody measured using SPR (Table 17). These antibodies were then tested in a cell-based potency assay using primary human monocyte-derived dendritic cells and autologous αGalCer-expanded NKT cells (Tables 18 and 19).

TABLE 17

| IgG | K_{D} (pM) |
|---|---|
| 402.8 | 145 |
| 402.8.53 | 578 |
| 402.8.54 | 870 |
| 402.8.55 | 878 |
| 402.8.60 | 198 |
| 402.8.84 | 587 |
| 402.8.86 | 357 |
| 402.8.87 | 344 |

TABLE 18

| | NKT IFN-γ | NKT IL-13 EC₅₀ |
|---|---|---|
| | EC₅₀ (ng/mL) | (ng/mL) |
| IgG | (moDC) | (moDC) |
| 402.8 | 0.105 | 0.154 |
| 402.8.53 | 0.658 | 1.524 |
| 402.8.60 | 0.244 | 0.189 |
| 402.8.84 | 0.516 | 0.663 |
| 42 | 116.0 | 17.54 |
| Negative | DNI | DNI |
| Control | | |

Note:
Negative Control - An irrelevant specificity IgG1;
DNI—Did Not Inhibit, where the inhibitory activity of the antibody was typically less than 50% of the maximal response by human NKT cells at 1 μg/mL;
moDC - primary monocyte-derived dendritic cells used as antigen presenting cells.

TABLE 19

| | NKT IFN-γ | NKT IL-4 | | |
|---|---|---|---|---|
| | EC₅₀ | EC₅₀ | NKT IFN-γ | NKT IL-4 EC₅₀ |
| | (ng/mL) | (ng/mL) | EC₅₀ (ng/mL) | (ng/mL) |
| Antibody | (moDC) | (moDC) | (moDC) | (moDC) |
| 402.8 | 14.96 | 22.45 | 4.358 | 0.273 |
| 402.8.53 | 277.2 | 105.4 | ND | ND |
| 402.8.54 | 137.6 | 109.1 | ND | ND |
| 402.8.55 | 444.6 | 175 | ND | ND |
| 402.8.60 | 24.68 | 26.8 | ND | ND |
| 402.8.84 | ND | ND | 30.64 | 0.593 |
| 402.8.86 | ND | ND | 14.31 | 0.721 |
| 402.8.87 | ND | ND | 25.91 | 1.008 |
| 42 | DNI | DNI | DNI | DNI |
| 51.1 | DNI | DNI | DNI | DNI |
| Negative | DNI | DNI | ND | ND |
| Control | | | | |

Note:
Negative Control - An irrelevant specificity IgG1;
DNI—Did Not Inhibit, where the inhibitory activity of the antibody was typically less than 50% of the maximal response by human NKT cells at 1 μg/mL;
moDC - primary monocyte-derived dendritic cells used as antigen presenting cells.

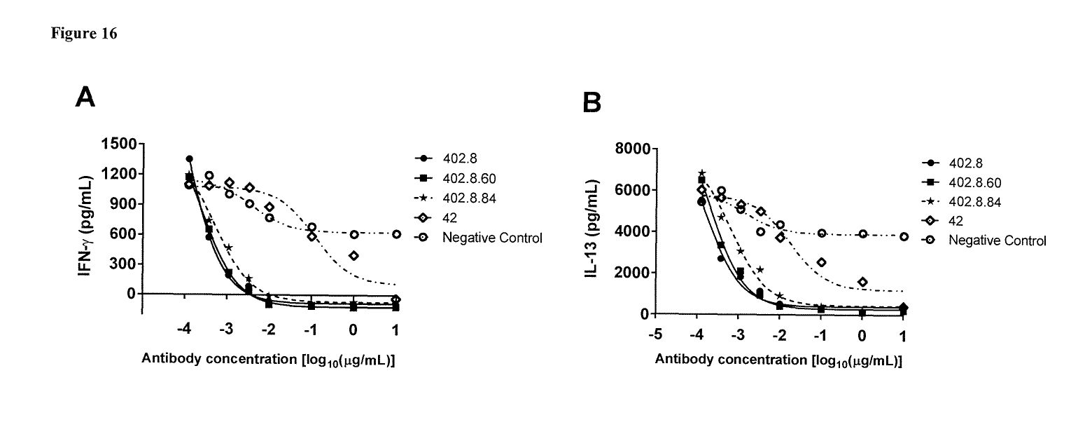

These variant antibodies derived from 402.8 demonstrated strong inhibition of αGalCer-mediated cytokine release by primary NKT cells. Some of the variant antibodies showed reduced potency compared with 402.8 whereas other antibodies retained similar potency as determined by dose-dependent inhibition of NKT cell-driven cytokine release. In several experiments, anti-CD1d antibodies 42 and 51.1 showed minimal inhibition such that a true EC50 value could not be calculated (Table 19). In experiments where the EC50 values could be determined, antibodies derived from 402.8 were of significantly improved potency compared with anti-CD1d antibody 42, which showed some inhibitory activity at the highest concentration of 10 μg/mL, but did not retain inhibitory activity at lower concentrations of antibody (FIG. 16 and Table 18). This result therefore demonstrates that the optimized anti-CD1d antibodies based on parental antibody 402.8 showed significant improvements in potent neutralizing activity compared with anti-CD1d antibodies 42 and 51.1 and similar neutralizing activity compared with 402.8 in the context of primary human cells that naturally express the CD1d antigen.

### Example 12

### Testing the Efficacy of Anti-CD1d Antibodies in Primary NKT Cell-Based Assays Using an Alternative Antigen to α-Galactosylceramide

The cell-based potency assays described in Example 6 employed αGalCer as the glycolipid antigen. Demonstrating that the anti-CD1d antibodies possess inhibitory activity in the context of an alternative glycolipid antigen to αGalCer supports the concept that such highly potent neutralizing anti-CD1d antibodies bind CD1d at a location away from the regions where lipid and/or glycolipids may be presented. The CD1d-restricted lipid and glycolipid antigens found in nature may differ from αGalCer in terms of chemical structure, and consequently it may be useful to demonstrate that the anti-CD1d antibodies described in the invention retain inhibitory activity in the context of a glycolipid antigen with a different chemical structure. In addition, it would be useful to characterize the inhibitory activity of potential self antigens, i.e., those found in a mammalian context.

Only a few glycolipid antigens with activity for human NKT cells have been characterized. These include iGb3 and lysophosphatidylcholine, however such antigens have only weak activation of NKT cells. The C24:1 N-acyl variant of an endogenous lipid, β-D-glucopyranosylceramide (hereafter known as C24:1 β-GluCer) was described to have activity for human NKT cells. A cell-based potency assay was developed to characterize the inhibitory activity of proprietary anti-CD1d antibodies in the context of this alternative antigen to αGalCer (Brennan, P. J., et al., 2011 Nat Immunol 12:1202-1211).

### Assay Methods and Results

The C24:1 N-acyl variant of β-D-glucopyranosylceramide (Avanti; D-glucosyl-β-1,1′ N-(15Z-tetracosenoyl)-D-erythro-sphingosine N-(15Z-tetracosenoyl)-1-β-glucosyl-sphing-4-ene) was solubilized in DMSO at 5 mg/mL at 37° C. for 2 hours before storing in small aliquots.

To perform the cell-based potency assays using C24:1 βGluCer, NKT cells were expanded with αGalCer as described in Example 6. Despite being stimulated in the presence of αGalCer, these NKT cells retained functional activity to C24:1 βGluCer, indicating that the TCR specificity of the NKT cell lines generated was also permissive to C24:1 β-GluCer recognition in the context of human CD1d. NKT cells were phenotyped by flow cytometry and only used in cell-based potency assays if the purity of the NKT cells exceeded 70%.

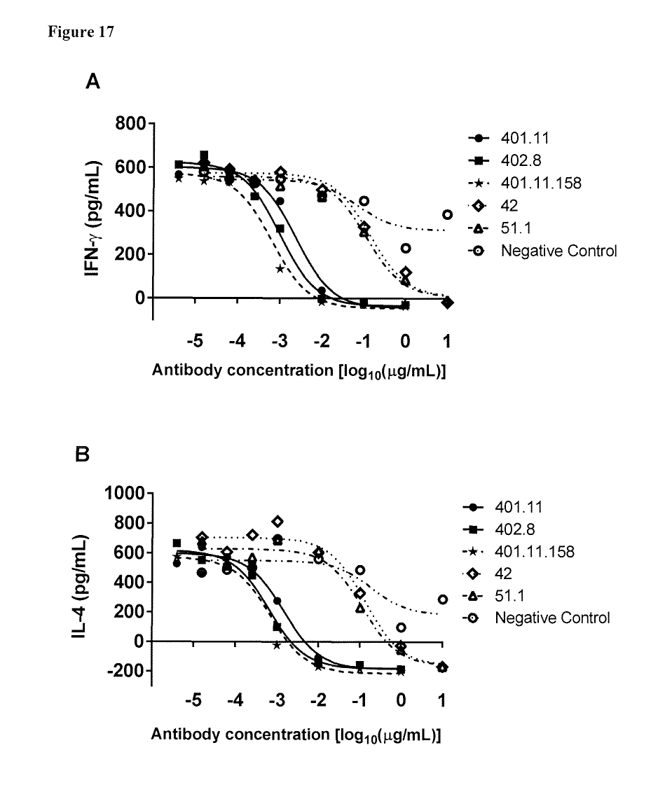

Monocyte-derived dendritic cells were generated as described in Example 6. These cells were cultured in 96-well flat bottom plates at 2×10⁴ cells per well and loaded with C24:1 βGluCer at 10 μg/mL for 24 hours. Inhibitory anti-CD1d antibodies 401.11.158, 401.11 and 402.8 were prepared in decreasing concentration from 1 μg/mL and 42, 51.1 and negative control antibodies were prepared in decreasing concentration from 10 μg/mL and then added to the C24:1 βGluCer-loaded dendritic cell cultures for 1 hr. Thereafter, NKT cells were added in a 1:1 ratio with the dendritic cells. Twenty-four hours later, cell-free supernatants were assayed for IFN-γ, IL-4, IL-5 IL-13 and TNF release. As an example, 401.11, 401.11.158 and 402.8 were tested in this assay. An irrelevant specificity negative control antibody was used a negative control and anti-CD1d antibodies 42 (BD Biosciences) and 51.1 (eBioscience) used as positive controls. The 42 and 51.1 antibodies and antibodies 401.11, 401.11.158 and 402.8 demonstrated inhibition of C24:1 βGluCer-induced cytokine release by primary human NKT cells in this primary cell-based assay (IFN-γ and IL-4 curves are shown in FIG. 17). In comparison, the negative control antibody demonstrated negligible inhibition of cytokine release by NKT cells.

The antibodies 42 and 51.1 showed some inhibition of cytokine release by NKT cells at high doses (10 μg/mL) but this effect was not sustained at lower doses. Compared with antibody 42, antibodies 401.11.158, 401.11, and 402.8 demonstrated up to 216-fold, up to 58-fold and up to 139-fold improved potency respectively (FIG. 17 and Table 20). Compared with antibody 51.1, antibodies 401.11.158, 401.11, and 402.8 demonstrated up to 175-fold, up to 47-fold and up to 112-fold improved potency respectively (FIG. 17 and Table 20; see IFN-γ assay EC₅₀ values).

TABLE 20

| | | | | IL-13 | TNF-α |
|---|---|---|---|---|---|
| | IFN-γ EC50 | IL-4 EC₅₀ | IL-5 EC₅₀ | EC₅₀ | EC₅₀ |
| | (ng/mL) | (ng/mL) | (ng/mL) | (ng/mL) | (ng/mL) |
| 401.11 | 2.376 | 1.452 | 1.231 | 5.549 | 1.793 |
| 402.8 | 0.9945 | 0.6364 | 0.9292 | 2.938 | 1.507 |
| 401.11.158 | 0.6397 | 0.6822 | 0.8679 | 1.439 | 0.8422 |
| 42 | 138.5 | 133.1 | 81.89 | 52.33 | 5.692 |
| 51.1 | 111.8 | 113.1 | 86.43 | 66.77 | 29.72 |
| Negative | DNI | DNI | DNI | DNI | DNI |
| Control | | | | | |

Negative Control: An antibody of an IgG4 isotype directed to a target other than CD1d;
DNI—Did Not Inhibit, where the inhibitory activity of the antibody was typically less than 50% of the maximal response by human NKT cells at 1 μg/mL.

### Example 13

### Antibodies Derived from 402.8 and 401.11 Share a Common Epitope on CD1d, which is not Shared by Anti-CD1d Antibodies

Variants of 402.8 and 401.11, and commercially sourced anti-CD1d antibodies were tested in a competition-based ELISA based on Example 7. First, it was shown that a biotinylated version of 402.8 competed with non-biotinylated antibody 401.11 but not with antibodies 42 and 51.1. This work is described below.

### Assay Methodology

Anti-CD1d antibody 402.8 was biotinylated using an EZ-link Sulfo-NHS-LC-biotin kit (Pierce) at a 3:1 ratio of biotin: 402.8. Free biotin was removed from the protein preparation by multiple washes with PBS and concentration by centrifugation (3000 rpm) through a centrifugal filter unit with a 30 kDa cutoff (Millipore). Maxisorp® ELISA plates (Nunc) were coated with 1.0 μg/mL human CD1d and incubated overnight at 4° C. Plates were then washed three times in PBS containing 0.1% Tween® 20, before the plate was blocked in 1% BSA for 1 hr at room temperature. Biotinylated 402.8 was then co-equilibrated for 5 minutes in a 1:1 ratio with non-biotinylated anti-CD1d antibodies. These antibodies were added to the plates for 1 hour at room temperature in two-fold decreasing concentrations from 40 μg/mL (i.e., a maximum of 200-fold excess compared with 0.2 μg/mL biotinylated 402.8), with a blank well at the final dilution (i.e., containing only biotinylated 402.8 antibody). Plates were then washed three times in PBS containing 0.1% Tween® 20. Streptavidin horseradish peroxidase conjugate (BD Biosciences) was added to the plates for 1 hour at room temperature in the dark. The plates were washed to remove unbound streptavidin-horseradish peroxidase. The assay signal was developed by incubating with 50 μL 3,3′,5,5′-Tetramethylbenzidine (KPL) and quenched with 50 μL 1 M HCl. Assay signals were read at A450 nm using a microplate reader (FluoStar Galaxy). Results were expressed as the raw A450 nm value and converted to degree of competition (percentage) values by subtracting the readings corresponding with zero percent inhibition from raw data.

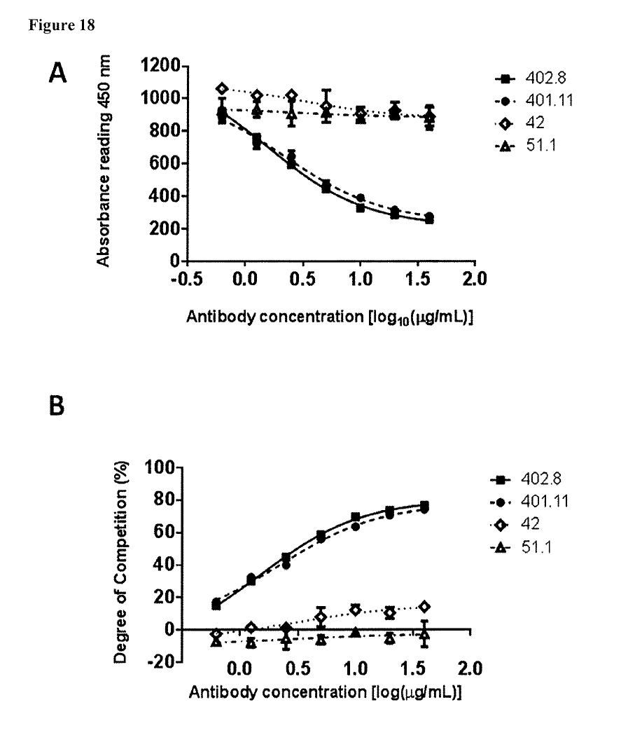

To establish that this method generated similar results as described in Example 7, biotinylated antibody 402.8 was competed with 401.11, and anti-CD1d antibodies 42 and 51.1 for binding to human CD1d. Under these assay conditions 402.8 and 401.11 competed for binding to human CD1d, as shown by absorbance values at 450 nm (FIG. 18A) and degree of competition with 402.8 (FIG. 18B), and consequently it was apparent that these antibodies shared an overlapping or common epitope on hCD1d. In contrast, 402.8 did not share an overlapping or common epitope with either 42 or 51.1. Taken together, this assay demonstrated that the highly potent anti-CD1d antibodies 402.8 and 401.11 bind to a similar high affinity neutralizing epitope on CD1d that is not shared by antibodies 42 and 51.1.

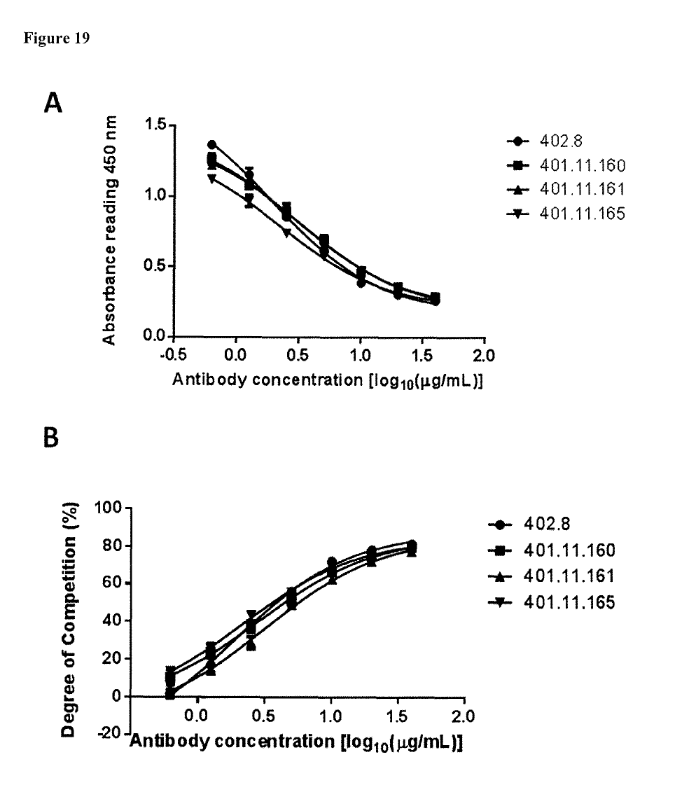

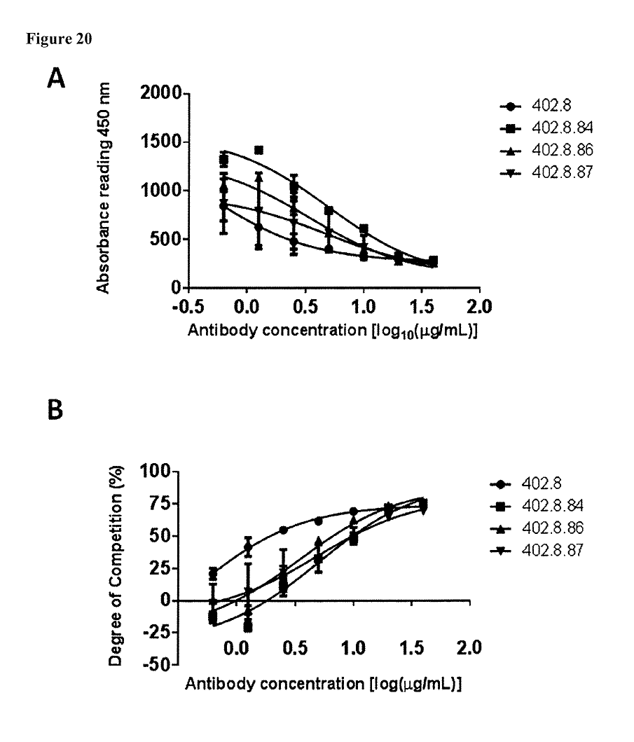

Using this modified assay, biotinylated antibody 402.8 was competed with the following antibodies (described in Example 11) for binding to recombinant human CD1d: 401.11.24, 401.11.26, 401.11.28, 401.11.86, 401.11.151, 401.11.152, 401.11.154, 401.11.155, 401.11.156, 401.11.157, 401.11.158, 401.11.159, 401.11.160, 401.11.161, 401.11.165, 401.11.166, 401.11.167, 401.11.179, 401.11.180, 401.11.181, 402.8.45, 402.8.53, 402.8.60, 402.8.84, 402.8.86 and 402.8.87. All of these antibodies competed with biotinylated 402.8 for binding to human CD1d, as shown by absorbance values at 450 nm and converted percentage competition with 402.8. In particular, antibodies 401.11.160, 401.11.161 and 401.11.165 strongly competed with biotinylated 402.8 as evidenced by Fig. demonstrating absorbance values at 450 nm (FIG. 19A) and degree of competition (FIG. 19B), as did antibodies 402.8.84, 402.8.86 and 402.8.87, as evidenced by figures demonstrating absorbance values at 450 nm (FIG. 20A) and degree of competition (FIG. 20B).

Testing Competition with Additional Anti-CD1d Antibodies

To investigate whether antibodies derived from 402.8 or 401.11 competed with other anti-CD1d antibodies, a total of 23 commercially-sourced anti-CD1d antibodies were tested. These antibodies included anti-human CD1d monoclonal antibodies, anti-mouse CD1d monoclonal antibodies, and polyclonal anti-human CD1d antibodies. The details of these antibodies are described in Table 21. Rat anti-mouse antibody hybridomas HB-321, HB-322, HB-323, HB-326 and HB-327 were sourced from American Type Culture Collection and passaged according to the supplier's instructions. Antibodies derived from these cell lines were purified by Protein G affinity chromatography and verified for binding to mouse CD1d (not shown).

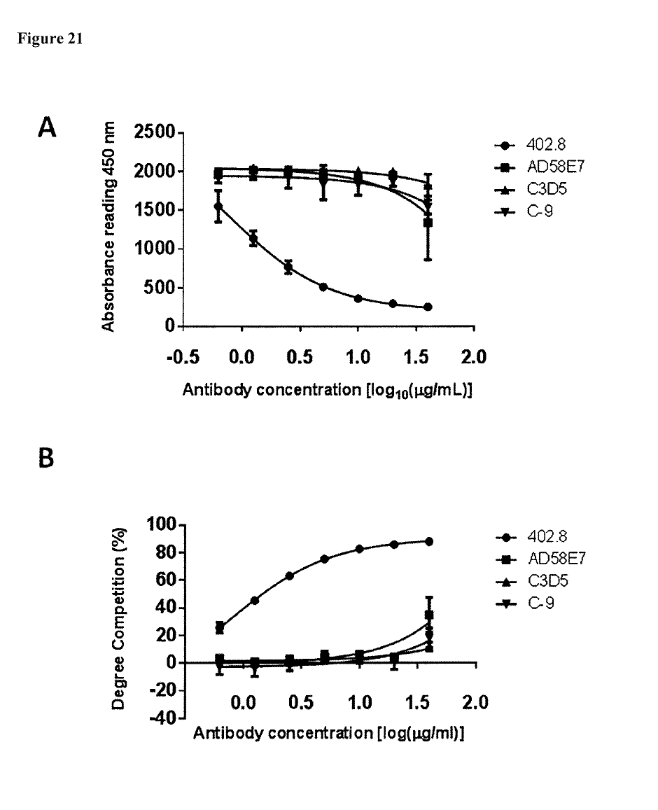

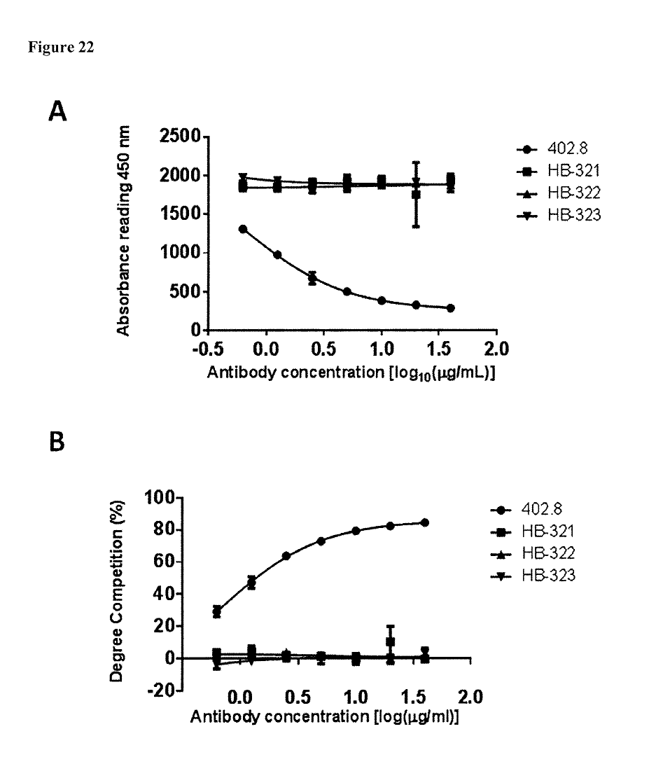

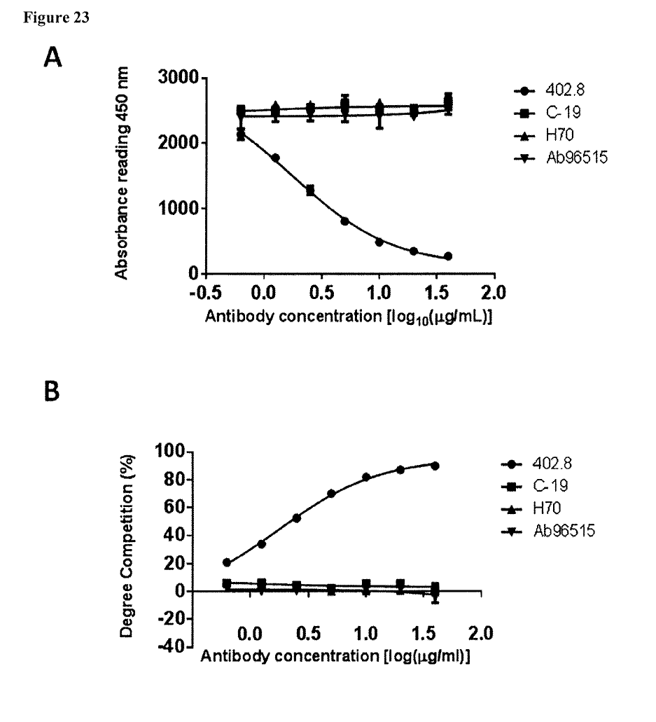

None of the 23 anti-CD1d antibodies described in Table 21 competed with 402.8 for binding to human CD1d, as shown by absorbance values at 450 nm and percentage competition with 402.8. Examples of these results are presented in FIG. 21-23. The anti-human CD1d antibodies AD58E7, C3D5 and C-9 did not compete with 402.8 for binding to human CD1d, as shown by absorbance readings at 450 nm (FIG. 21A) and converted degree of competition values (FIG. 21B). As another example of these results, anti-mouse CD1d antibodies HB-321, HB-322 and HB-323 did not compete with 402.8 for binding to human CD1d, as demonstrated by absorbance readings at 450 nm (FIG. 22A) and converted degree of competition values (FIG. 22B). Similarly, as an example the polyclonal anti-human CD1d antibodies C-19, H70 and Ab96515 did not compete with 402.8 for binding to human CD1d, as shown by absorbance readings at 450 nm (FIG. 23A) and converted degree of competition values (FIG. 23B). It is notable that none of the polyclonal anti-CD1d antibodies tested competed with 402.8 for binding to human CD1d. These data demonstrate that the highly potent anti-CD1d antibody 402.8, as an example of the highly potent novel anti-CD1d antibodies tested, bound to a similar high affinity neutralizing epitope that is not shared by the extensive list of anti-CD1d antibodies tested.

TABLE 21

| | Clone or | | | |
|---|---|---|---|---|
| Antibody | Catalog | | | |
| Number | Number | Isotype | Specificity | Source |
| 1 | NOR3.2 | Mouse | Human CD1d | Pierce |
| | | monoclonal | | |
| 2 | AD5-8E7 | Mouse | Human CD1d | Miltenyi Biotec |
| | | monoclonal | | |
| 3 | C3D5 | Mouse | Human CD1d | Santa Cruz Biotechnology |
| | | monoclonal | | |
| 4 | C-9 | Mouse | Human CD1d | Santa Cruz Biotechnology |
| | | monoclonal | | |
| 5 | G10 | Mouse | Human CD1d | Santa Cruz Biotechnology |
| | | monoclonal | | |
| 6 | 3H649 | Mouse | Human CD1d | Santa Cruz Biotechnology |
| | | monoclonal | | |
| 7 | LS-C122839 | Mouse | Human CD1d | Lifespan Biosciences, Inc. |
| | | monoclonal | | |
| 8 | LS-C4448 | Mouse | Human CD1d | Lifespan Biosciences, Inc. |
| | | monoclonal | | |
| 9 | LS-C4449 | Mouse | Human CD1d | Lifespan Biosciences, Inc. |
| | | monoclonal | | |
| 10 | LS-C122840 | Rat | Mouse CD1d | LifeSpan Biosciences, Inc. |
| | | monoclonal | | |
| 11 | 1B1 | Rat | Mouse CD1d | BD Biosciences |
| | | monoclonal | | |
| 12 | HB-321 | Rat | Mouse CD1d | American Type Culture |
| | (clone 19F8) | monoclonal | | Collection |
| 13 | HB-322 | Rat | Mouse CD1d | American Type Culture |
| | (clone 15F7) | monoclonal | | Collection |
| 14 | HB323 (clone | Rat | Mouse CD1d | American Type Culture |
| | 20H2) | monoclonal | | Collection |
| 15 | HB-326 | Rat | Mouse CD1d | American Type Culture |
| | (clone 15C6) | monoclonal | | Collection |
| 16 | HB-327 | Rat | Mouse CD1d | American Type Culture |
| | (clone 4C4) | monoclonal | | Collection |
| 17 | 3C11 | Rat | Mouse CD1d | BD Biosciences |
| | | monoclonal | | |
| 18 | K253 | Mouse | Mouse CD1d | Biolegend |
| | | monoclonal | | |
| 19 | C-19 | Goat | Human CD1a, | Santa Cruz Biotechnology |
| | | Polyclonal | CD1b, CD1d | |
| 20 | H70 | Rabbit | Human CD1d | Santa Cruz Biotechnology |
| | | Polyclonal | | |
| 21 | Ab96515 | Rabbit | Human CD1d | AbCam |
| | | Polyclonal | | |
| 22 | GTX104898 | Rabbit | Human CD1d | GeneTex |
| | | Polyclonal | | |
| 23 | 1401052 | Mouse | Human CD1d | Sigma Aldrich |
| | | Polyclonal | | |

As an adjunct to the above examples, extended experiments were conducted using a biotinylated version of an antibody derived from 401.11. This experiment was done to demonstrate that an antibody which competes with 402.8 for binding to human CD1d could also be used as a biotinylated reagent in an amended assay, and show the same result. In the following description, the assay was amended such that 401.11.158, an example of the 401.11-derived antibodies, was biotinylated and competed with 402.8 and other anti-CD1d antibodies 42 and 51.1.

### Assay Methodology

Anti-CD1d antibody 401.11.158 was biotinylated using an EZ-link Sulfo-NHS-LC-biotin kit (Pierce) at a 3:1 ratio of biotin: 401.11.158. Free biotin was removed from the protein preparation by multiple washes with PBS and concentration by centrifugation (3000 rpm) through a centrifugal filter unit with a 30 kDa cutoff (Millipore). Anti-CD1d antibody Maxisorp® ELISA plates (Nunc) were coated with 1.0 μg/mL human CD1d and allowed to incubate overnight at 4° C. Plates were then washed three times in PBS containing 0.1% Tween® 20, before the plate was blocked in 1% BSA for 1 hr at room temperature. Biotinylated 401.11.158 was then co-equilibrated for 5 minutes in a 1:1 ratio with non-biotinylated anti-CD1d antibodies (401.11.158, 402.8, 401.11, 42 and 51.1). These antibodies were added to the plates for 1 hour at room temperature in decreasing concentrations from 40 μg/mL (i.e. a maximum of 200-fold excess compared with 0.2 μg/mL biotinylated 401.11.158). Plates were then washed three times in PBS containing 0.1% Tween20. Streptavidin horseradish peroxidase conjugate (BD Biosciences) was added to the plates for 1 hour at room temperature in the dark. The plates were washed to remove unbound streptavidin-horseradish peroxidase. The assay signal was developed by incubating with 50 μL 3,3′,5,5′-Tetramethylbenzidine (KPL) and quenched with 50 μL 1 M HCl. Assay signals were read at A450 nm using a microplate reader (FluoStar Galaxy). Results were expressed as the raw A450 nm value and converted to degree of competition (percentage) values by subtracting the readings corresponding with zero percent inhibition from raw data.

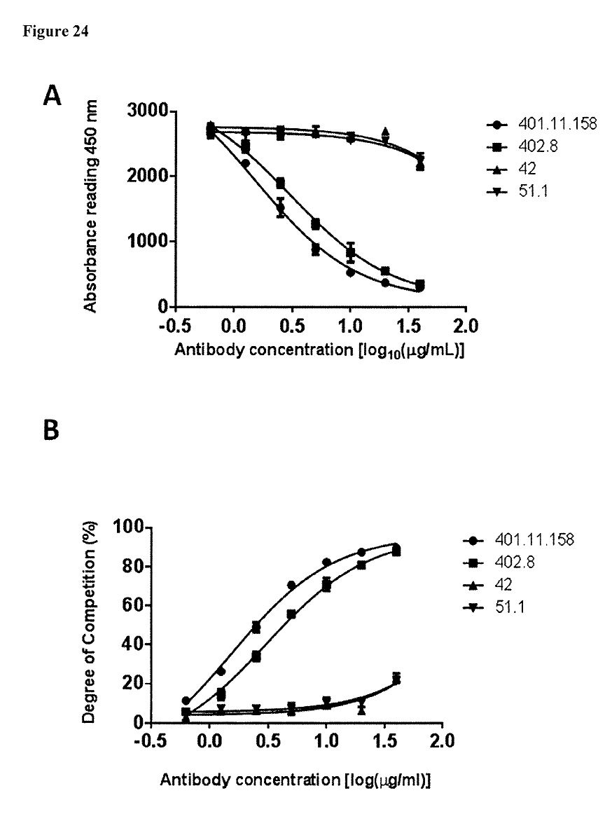

Using the above described method, 401.11.158 and 402.8 competed for binding to human CD1d, as shown by absorbance values at 450 nm (FIG. 24A) and degree of competition with 401.11.158 (FIG. 24B) and therefore share an overlapping or common epitope. In contrast, 401.11.158 does not share an overlapping or common epitope with either 42 or 51.1. Taken together, these data demonstrate that the highly potent anti-CD1d antibodies 401.11.158 and 402.8 may bind to a similar high affinity neutralizing epitope that is not shared by prior art antibodies 42 and 51.1 with lesser potency.

### Example 14

### Epitope Mapping Experiments

### Methods

### Preparation of FAbs

FAbs of anti-CD1d antibodies, 402.8 and 401.11.165 were prepared by Papain digest using the FAb Preparation Kit (Pierce) according to the manufacturer's instructions. The intact FAb was removed from Fc (Fragment crystallisable) containing protein by running the sample over a Protein A column equilibrated with Phosphate buffered saline (1×PBS) pH 7.0 and collecting the flow-through. The FAbs were then analysed by size exclusion chromatography (SEC) using a TSK gel G3000SW×1 column (TOSOH) at 0.5 ml/min with 1×PBS as a running buffer. The results indicated that the FAbs were >95% pure.

### FAb-CD1d Binding ELISA

To confirm binding of the FAbs to human CD1d, an ELISA was performed in which human CD1d was coated at 1 μg/mL in PBS onto a Maxisorp® plate (NUNC) overnight at 4° C. The wells were then washed with 3 separate washes with 1×PBS with 0.05% Tween-20 (Sigma). The wells were blocked with 1% BSA in PBS for 1 hour at room temperature. The wells were then washed with 1×PBS as described above. A titration of FAb or full-length antibody was then performed starting from a concentration of 10 μg/mL and dilutions performed at 1:4 across the plate. A PBS only control was included. The plate was incubated for 1 hour at room temperature and then the well were washed as described previous. 100 μL per well of secondary antibody (Goat Anti-human Kappa F+B HRPO Conjugate, Invitrogen) was added at a dilution of 1:2000 in 1×PBS and incubated for 1 hour at room temperature, and then the wells were washed as described previously. 50 μL of TMB (Sigma) was added and the plate was incubated until colour development. The reaction was stopped by adding 50 μL of 1M HCl to each well. The absorbance was read at 450 nm (referenced at 620 nm).

### H/D Exchange Experiments

Human CD1d was diluted in 1×PBS (pH 7) to a concentration of 12.8 μM. It was then mixed with a FAb fragment of 402.8 or 401.11.165 at a concentration of 14.1 μM. 14 μL of this solution was mixed with 264 of 50 mM Phosphate pH 7 in D2O (where D is deuterium). Separate solutions were prepared and incubated for 30, 100, 300 or 1000s at 23° C. At the end of the incubation period 20 μL of 2 M urea, 1M TCEP pH 3.0 was added to each solution. The sample was then passed over an immobilized pepsin column at 200 μL/min in 0.05% trifluoracetic acid (TFA) in H₂O. Peptic fragments were loaded onto a reversed-phase trap column and desalted with 0.05% TFA in H2O for 3 mins. Peptides were separated by a C18 column with a linear gradient of 13% to 35% of buffer comprising 95% acetonitrile, 5% H2O, 0.0025% TFA) over 23 mins. The peptides were then analysed by mass spectrometry in profile mode. Fully deuterated control samples were prepared by mixing 32.2 μL of 0.615 mg/mL CD1d with 59.8 μL of 100 mM TCEP in D2O and incubate at 60° C. for 3 hours.

### CD1d Mutein ELISA

All solutions were added in 50 μL/well. CD1d (wild-type) and related constructs (such as muteins or CD1d with amino acid substitutions) were coated on 96-well Maxisorp® ELISA Plates (NUNC) at 1 μg/mL in PBS overnight at 4° C. The wells were then washed with wash buffer (PBS+0.05% Tween-20) 3 times. The plates were blocked for 1 hr at room temperature in blocking buffer (PBS+1% BSA) before being washed 3 times. Antibody in antibody diluent (PBS+1% BSA+0.05% Tween-20) was added to the wells in a half log titration starting from 10 μg/ml, and no antibody (0 μg/mL) was included as a negative control. The plate was then incubated at room temperature for 1 hour. The plate was then washed as described above. Secondary antibodies (HRP-goat anti human IgG (H+L), Invitrogen) was added at 1:2000 dilution in antibody diluent and incubated for 1 hour at room temperature. After washing the plate, 50 μL of TMB (Sigma) was added to each well. 50 μL of 1M HCl was added after colour development to stop the reaction. The absorbance of each well of the plate was read at 450 nm (referenced at 620 nm).

### Results:

### Production of FAbs of 402.8 and 401.11.165

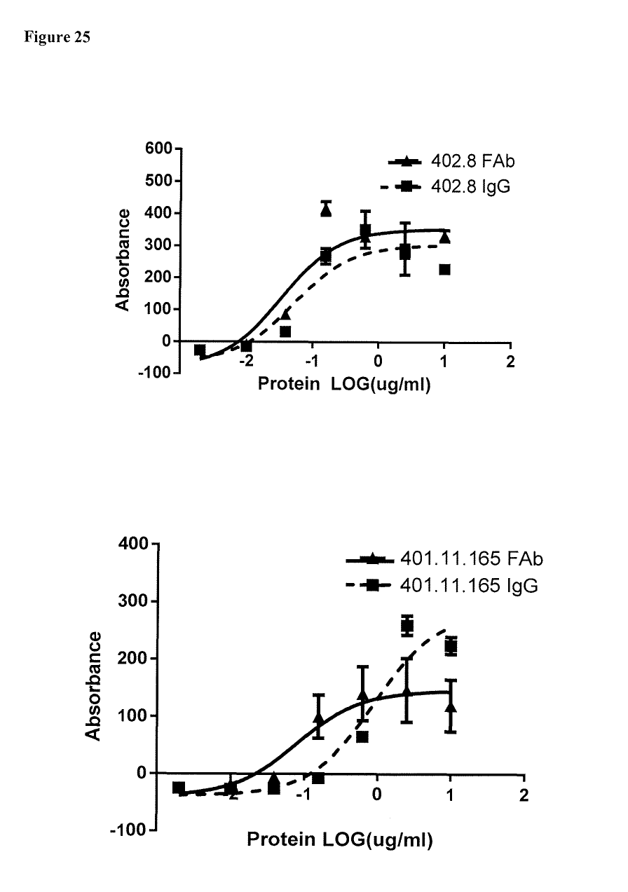

Using papain digest, FAbs of 402.8 and 401.11.165 were isolated. As measured by size exclusion chromatography (SEC) the constructs had a purity of >95%. When tested in an ELISA the FAbs retained binding to human CD1d (FIG. 25).

### H/D Exchange Experiments

Using the method for H/D exchange described above, antibodies 402.8 and 401.11.165, as FAbs, were complexed to human CD1d, then incubated in D2O. This exchanges hydrogen for deuterium on the CD1d molecule except in the locations in which the antibody has bound to the CD1d and to which the D2O has no access. Separately human CD1d was incubated with D2O in the absence of antibody. CD1d from both samples underwent trypic digest and was then analysed via mass spectrometry. The difference in deuteration levels in each segment of CD1d on exchange experiments with and without FAbs 402.8 and 401.11.165 are shown in the following Tables 22 and 23 respectively. The sequence of the extracellular domain of CD1d used in these experiments is listed below:

(SEQ ID No: 19)
EVPQRLFPLRCLQISSFANSSWTRTDGLAWLGELQTHSWSNDSDTVRSLK
PWSQGTFSDQQWETLQHIFRVYRSSFTRDVKEFAKMLRLSYPLELQVSAG
CEVHPGNASNNFFHVAFQGKDILSFQGTSWEPTQEAPLWVNLAIQVLNQD
KWTRETVQWLLNGTCPQFVSGLLESGKSELKKQVKPKAWLSRGPSPGPGR
LLLVCHVSGFYPKPVWVKWMRGEQEQQGTQPGDILPNADETWYLRATLDV
VAGEAAGLSCRVKHSSLEGQDIVLYWGGSYTSGSLVPRGSGSKRVHHHHH
HHH

TABLE 22
Difference in deuteration levels in each segment of CD1d on exchange
experiments with and without FAb 402.8

| | |
|---|---|
| Peptide | Incubation Time (s) |

| Start | End | 30 | 100 | 300 | 1,000 | Avg |
|---|---|---|---|---|---|---|
| 3 | 11 | −3% | −1% | −2% | 0% | −2% |
| 3 | 16 | −3% | −3% | 0% | −3% | −2% |
| 12 | 16 | −2% | −6% | 4% | −8% | −3% |
| 16 | 18 | 5% | −12% | −18% | −9% | −9% |
| 16 | 28 | −3% | −9% | −15% | −10% | −9% |
| 19 | 28 | −6% | −8% | −13% | −10% | −9% |
| 31 | 34 | −2% | 0% | −1% | 2% | 0% |
| 31 | 36 | 1% | −4% | −1% | 1% | −1% |
| 35 | 36 | 7% | −12% | 0% | −1% | −2% |
| 48 | 61 | −2% | 1% | −1% | 2% | 0% |
| 48 | 62 | −1% | 2% | 2% | 2% | 1% |
| 64 | 76 | 3% | −2% | 2% | −1% | 1% |
| 65 | 76 | 4% | −2% | 0% | −1% | 0% |
| 79 | 86 | −1% | −6% | −14% | −22% | −11% |
| 89 | 94 | −50% | −46% | −44% | −44% | 46% |
| 89 | 95 | −58% | −61% | −58% | −52% | −57% |
| 114 | 119 | 0% | −9% | −19% | −16% | −11% |
| 114 | 123 | −3% | −6% | −12% | −10% | −8% |
| 115 | 123 | −5% | −11% | −15% | −12% | −11% |
| 120 | 123 | −7% | −2% | −1% | −2% | −3% |
| 126 | 139 | −7% | −3% | −3% | −3% | −4% |
| 126 | 142 | −13% | −9% | −10% | −9% | −10% |
| 141 | 142 | −43% | −45% | −36% | −25% | −37% |
| 145 | 155 | −7% | −8% | −9% | −10% | −9% |
| 146 | 155 | −7% | −5% | −16% | −13% | −10% |
| 158 | 172 | −3% | −5% | −6% | −7% | −5% |
| 175 | 188 | 2% | 1% | −2% | −1% | 0% |
| 175 | 190 | 2% | 2% | −3% | 1% | 0% |
| 191 | 201 | −1% | 1% | −3% | 1% | −1% |
| 193 | 201 | 2% | −1% | −4% | 3% | 0% |
| 204 | 216 | 1% | 0% | 2% | 0% | 1% |
| 206 | 216 | 2% | 1% | 4% | 0% | 2% |
| 219 | 224 | 1% | 1% | 1% | 2% | 1% |
| 219 | 239 | 1% | 2% | −2% | 1% | 0% |
| 219 | 242 | 0% | 1% | −1% | 1% | 0% |
| 225 | 242 | 0% | 1% | −2% | 0% | 0% |
| 244 | 248 | 1% | 0% | −2% | 0% | 0% |
| 245 | 248 | 1% | 1% | −1% | 0% | 0% |
| 251 | 259 | 3% | −1% | 0% | 4% | 1% |
| 251 | 271 | 0% | 3% | −2% | −1% | 0% |
| 261 | 271 | −3% | −4% | −2% | 2% | −2% |
| 274 | 303 | −3% | 0% | −2% | 0% | −1% |
| 277 | 303 | −5% | 0% | −2% | 0% | −2% |
| 326 | 332 | −2% | 1% | −2% | 0% | −1% |
| 326 | 345 | 0% | 0% | −2% | 0% | −1% |
| 335 | 345 | −1% | 0% | −2% | 1% | −1% |
| 348 | 359 | 0% | −1% | 0% | −1% | −1% |
| 351 | 359 | 1% | −2% | 0% | 1% | 0% |
| 360 | 362 | 1% | 4% | 1% | 1% | 1% |
| 360 | 377 | 0% | 1% | −4% | −3% | −1% |
| 365 | 377 | 0% | 0% | −2% | 2% | 0% |
| 380 | 385 | −3% | −4% | −4% | −1% | −3% |
| 382 | 385 | −5% | −3% | −3% | −2% | −3% |
| 388 | 389 | 0% | 0% | 0% | −1% | 0% |
| 388 | 390 | 0% | 0% | −1% | −1% | 0% |
| 392 | 401 | 3% | 1% | −2% | 1% | 1% |
| 392 | 402 | 3% | 1% | −3% | 0% | 0% |
| 404 | 422 | −1% | 0% | −2% | 0% | −1% |
| 406 | 422 | 0% | 0% | −3% | −2% | −1% |

The mean±standard deviation (S.D.) % deuterium difference was calculated across 50% of the peptides with the lowest % deuterium difference. A value lower then the mean−3 S.D. was considered significant. The mean (50%)−3 S.D. % deuterium difference across the data set was 0%. The CD1d sequences that had the greatest protected regions upon complexing with 402.8 were:

89-95 of SEQ ID NO: 116 - LSYPLEL
141-142 of SEQ ID NO: 116 - NL

TABLE 23
Difference in deuteration levels in each segment of CD1d on exchange
experiments with and without FAb 401.11.165

| | |
|---|---|
| Peptide | Incubation Time (s) |

| Start | End | 30 | 100 | 300 | 1,000 | Avg |
|---|---|---|---|---|---|---|
| 3 | 11 | 1% | −1% | −1% | −1% | 0% |
| 3 | 16 | 2% | −3% | 2% | −2% | 0% |
| 12 | 16 | 3% | −6% | 5% | −3% | 0% |
| 16 | 18 | 3% | −1% | 4% | 3% | 2% |
| 16 | 28 | 1% | 0% | 0% | 0% | 0% |
| 19 | 28 | 1% | 0% | −1% | −1% | 0% |
| 31 | 34 | 1% | 0% | 0% | 1% | 1% |
| 31 | 36 | 1% | 0% | 1% | −1% | 0% |
| 35 | 36 | 1% | −1% | 1% | −6% | −1% |
| 48 | 61 | — | — | — | — | — |
| 48 | 62 | 4% | 0% | 0% | −4% | 0% |
| 64 | 76 | 6% | 2% | 5% | 2% | 4% |
| 65 | 76 | 4% | 2% | 3% | −1% | 2% |
| 79 | 86 | 2% | 2% | −3% | −5% | −1% |
| 89 | 94 | −14% | −10% | −12% | −19% | −14% |
| 89 | 95 | — | — | — | — | — |
| 114 | 119 | — | — | — | — | — |
| 114 | 123 | 6% | 9% | 6% | 6% | 7% |
| 115 | 123 | 11% | 10% | 9% | 10% | 10% |
| 120 | 123 | — | — | — | — | — |
| 126 | 139 | −13% | −14% | −10% | −10% | −12% |
| 126 | 142 | −23% | −19% | −19% | −22% | −20% |
| 141 | 142 | −69% | −70% | −61% | −67% | −67% |
| 145 | 155 | 0% | 4% | −2% | −6% | −1% |
| 146 | 155 | — | — | — | — | — |
| 158 | 172 | 4% | 6% | 5% | 1% | 4% |
| 175 | 188 | 1% | 3% | 1% | 0% | 1% |
| 175 | 190 | 2% | 1% | −3% | −2% | 0% |
| 191 | 201 | 1% | 1% | −1% | 0% | 0% |
| 193 | 201 | 4% | 2% | −4% | −2% | 0% |
| 204 | 216 | 1% | 2% | 1% | 0% | 1% |
| 206 | 216 | 3% | 2% | 2% | −1% | 2% |
| 219 | 224 | 2% | 2% | 2% | 2% | 2% |
| 219 | 239 | −1% | 2% | 0% | −1% | 0% |
| 219 | 242 | 2% | 1% | 0% | −1% | 0% |
| 225 | 242 | 2% | 0% | −1% | −2% | 0% |
| 244 | 248 | 1% | 0% | −1% | 0% | 0% |
| 245 | 248 | 2% | 1% | −1% | −1% | 0% |
| 251 | 259 | 6% | 0% | 2% | −1% | 2% |
| 251 | 271 | 0% | 4% | 0% | −4% | 0% |
| 261 | 271 | 1% | −2% | 3% | 4% | 1% |
| 274 | 303 | 1% | 0% | 0% | −1% | 0% |
| 277 | 303 | 0% | 0% | −1% | −1% | 0% |
| 326 | 332 | 2% | 1% | −1% | −2% | 0% |
| 326 | 345 | 2% | 0% | −1% | −1% | 0% |
| 335 | 345 | 2% | −1% | −1% | −1% | 0% |
| 348 | 359 | 3% | −1% | 1% | −3% | 0% |
| 351 | 359 | 2% | 0% | 1% | −2% | 0% |
| 360 | 362 | 2% | 4% | 2% | −1% | 2% |
| 360 | 377 | 2% | 1% | 0% | −1% | 1% |
| 365 | 377 | 3% | 0% | −1% | 0% | 1% |
| 380 | 385 | −2% | −5% | −4% | −4% | −4% |
| 382 | 385 | −3% | −3% | −3% | −3% | −3% |
| 388 | 389 | 0% | 0% | 0% | −1% | 0% |
| 388 | 390 | 1% | 0% | 0% | −1% | 0% |
| 392 | 401 | 5% | 1% | −1% | −1% | 1% |
| 392 | 402 | 4% | 1% | −3% | −1% | 0% |
| 404 | 422 | 2% | 0% | 0% | −1% | 0% |
| 406 | 422 | 3% | 1% | −1% | −1% | 0% |

Similarly for 401.11.165, the mean±standard deviation (S.D.) % deuterium difference was calculated across 50% of the peptides with the lowest % deuterium difference. A value lower then the mean−3 S.D. was considered significant. The mean (50%)−3 S.D. % deuterium difference across the data set was −5%. The CD1d sequences that had the greatest protected regions upon complexing with 401.11.165 were:

89-94 of SEQ ID NO 116 - LSYPLE
126-142 of SEQ ID NO 116 - QGTSWEPTQEAPLWVNL

Though having different primary amino acid sequences, both 401.11.165 and 402.8, when bound to human CD1d, protect similar regions of the molecule. These regions, collectively known as the epitope, include the region of CD1d around the sequence LSYPLE (89-94 of SEQ ID NO 116). The region QGTSWEPTQEAPLWVNL (126-142 of SEQ ID NO 116) is also protected in the above H/D exchange experiments and several amino acids, NL (141-142 of SEQ ID NO 116), within this larger region are highly protected.

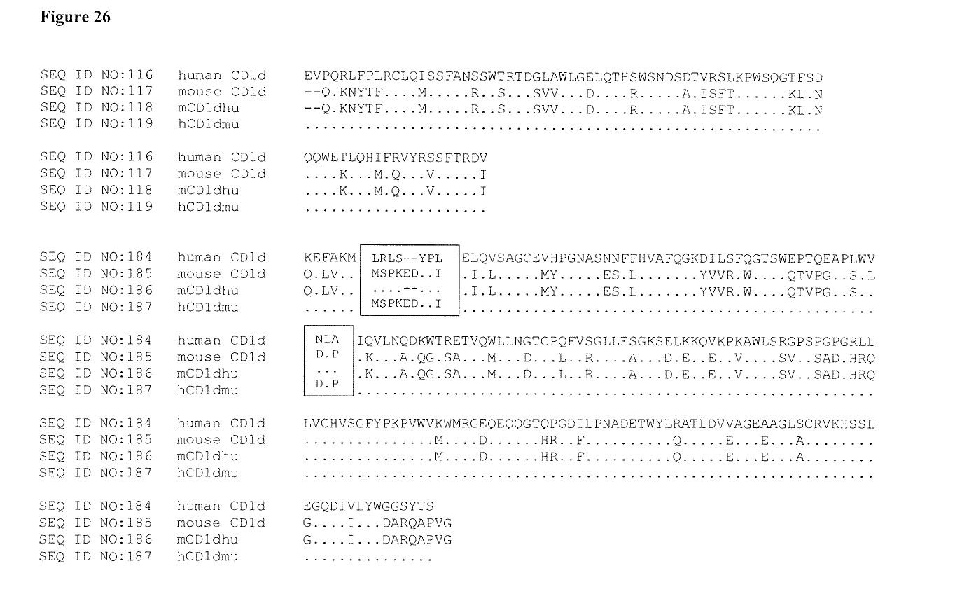

In order to confirm if the sequence LSYPLE (89-94 of SEQ ID NO 116) and NL (141-142 of SEQ ID NO 116) on human CD1d were important for the binding of the anti-CD1d antibodies described within, a series of CD1d constructs were produced. These constructs are listed in FIG. 26 and are described below:
hCD1dmu (SEQ ID NO:119)—Human CD1d in which amino acids located between positions 87 to 93 (LRLSYPL) and 141 to 143 (NLA) have been replaced with murine CD1d sequences (MSPKEDYPI and DLP respectively) with positions numbered according to human CD1d (SEQ ID NO:116).
mCD1dhu (SEQ ID NO:118)—Murine CD1d in which the amino acids located between positions 85 to 93 (MSPKEDYPI) and 141 to 143 (DLP) have been replaced with human CD1d sequence (LRLSYPL and NLA respectively) with positions numbered according to murine CD1d (SEQ ID NO:117).

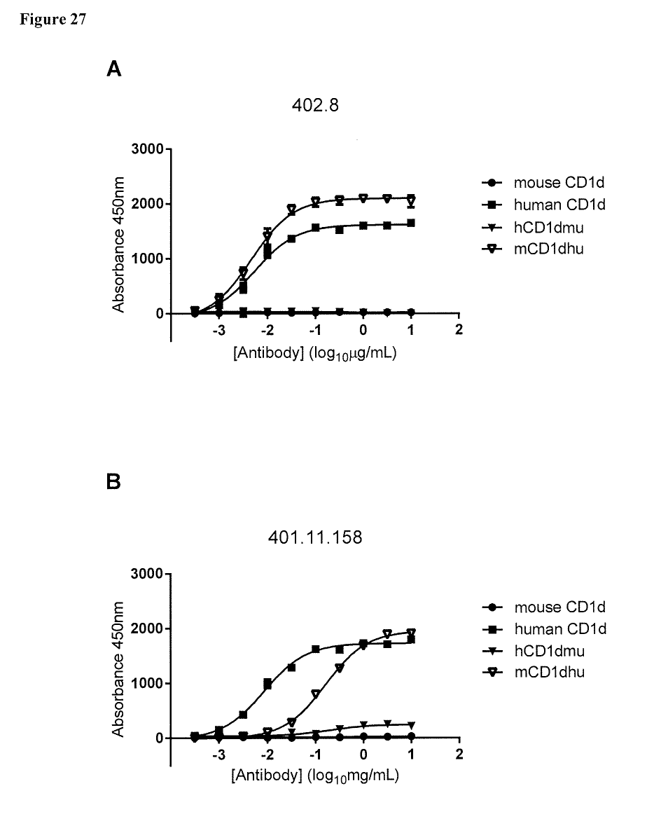

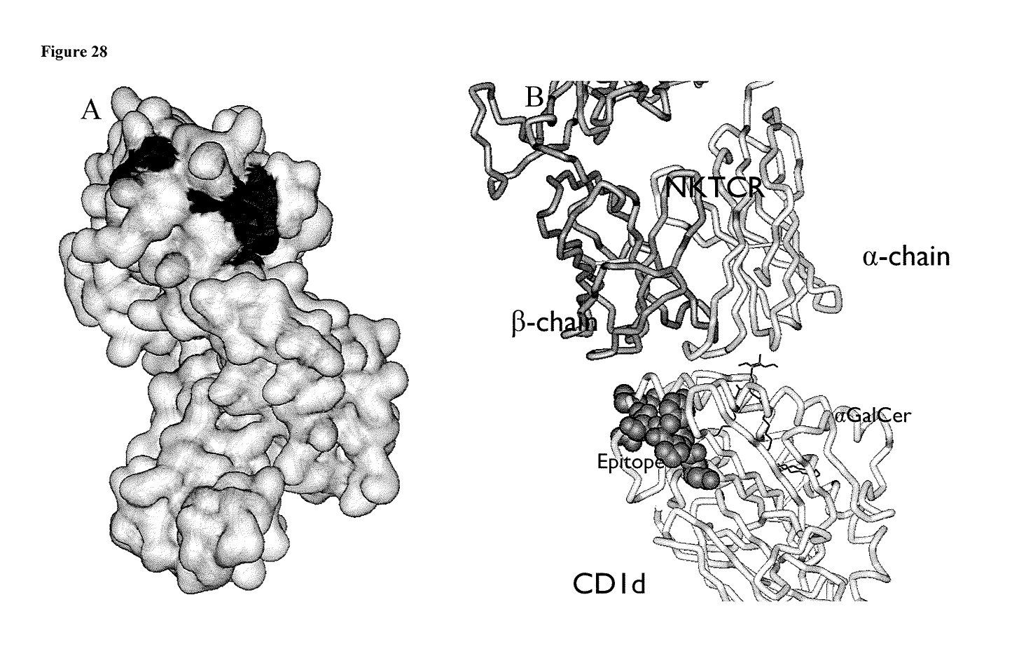

All CD1d constructs were expressed in HEK-293E cells and were detected by polyclonal antibodies against human or mouse CD1d in an ELISA format. Human CD1d, mouse CD1d, mCD1dhu and hCD1dmu were coated onto an ELISA plate and the binding of antibodies 402.8 and 401.11.158 to these CD1d constructs was determined. Both antibodies bound to human CD1d but did not bind to mouse CD1d (FIG. 27). They both bound to mouse CD1d into which the human sequence had been introduced (mCD1dhu) indicating these human sequence amino acids are crucial for the binding of the antibodies to human CD1d (FIG. 27). Likewise, when these amino acids on human CD1d were replaced by mouse sequence at the corresponding locations (hCD1dmu), the antibodies no longer bound this CD1d construct (FIG. 27). Taken together these results indicate that the sequence of human CD1d between 89-95 and 141-143 are within the epitope to which these anti-CD1d antibodies bind.

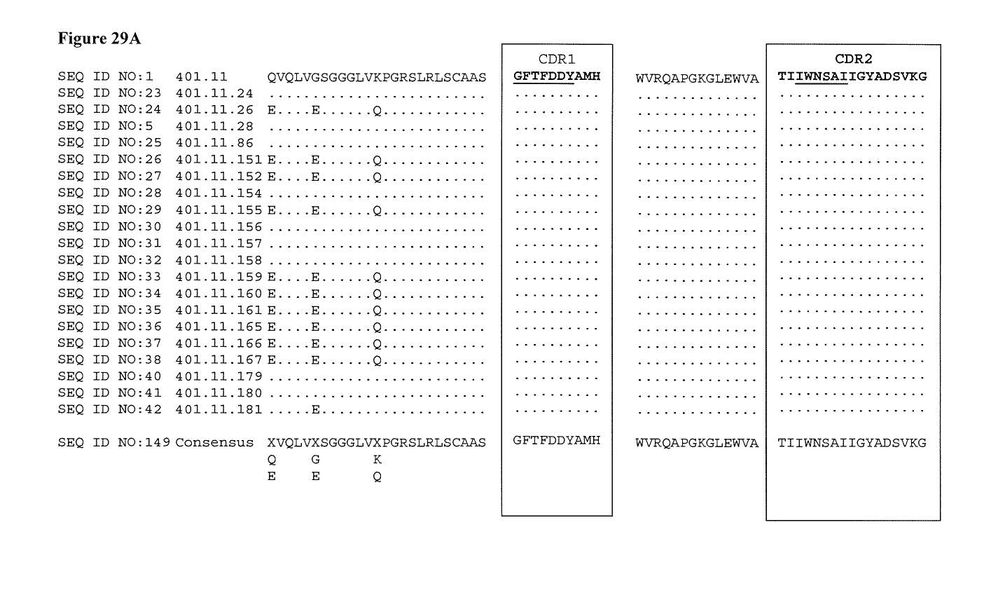

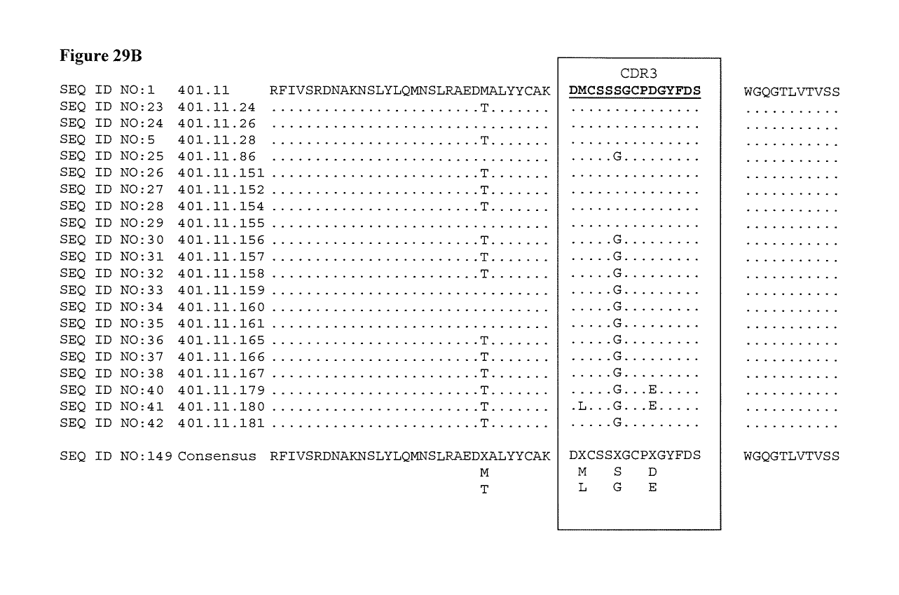

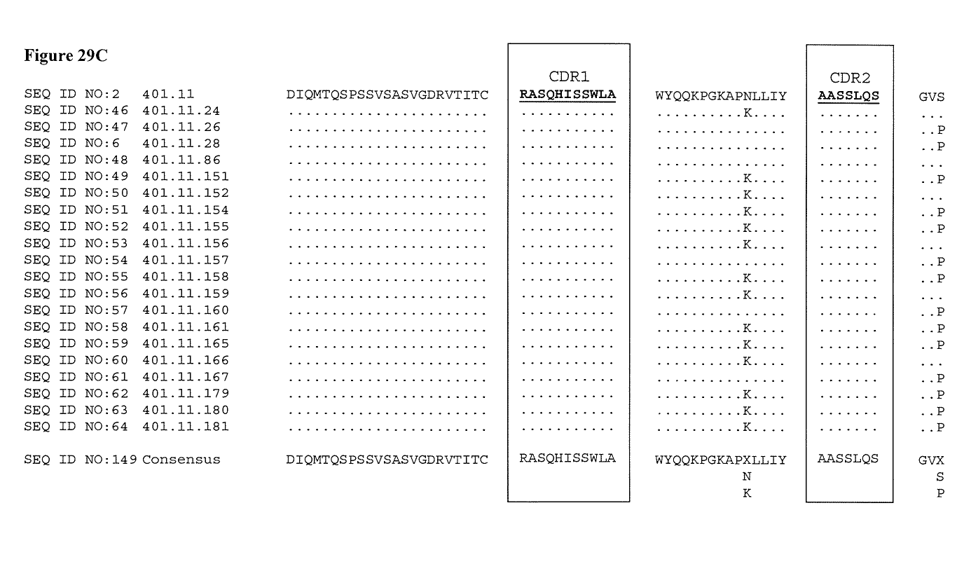

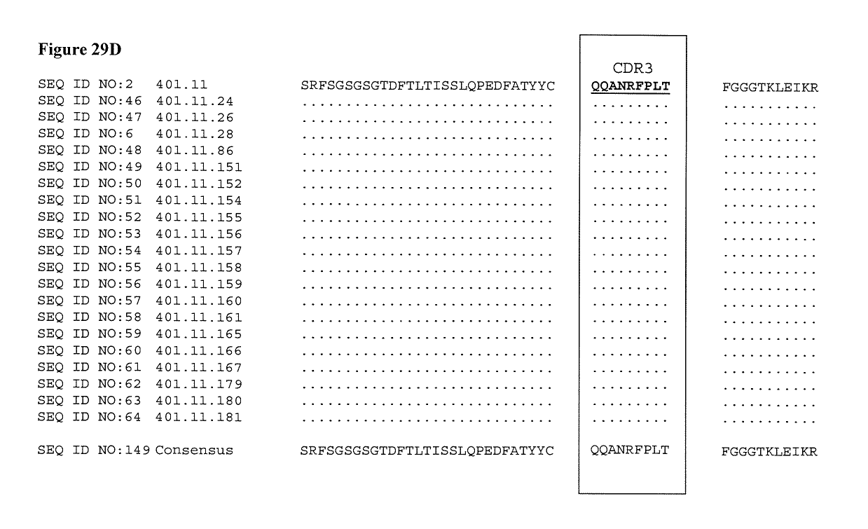

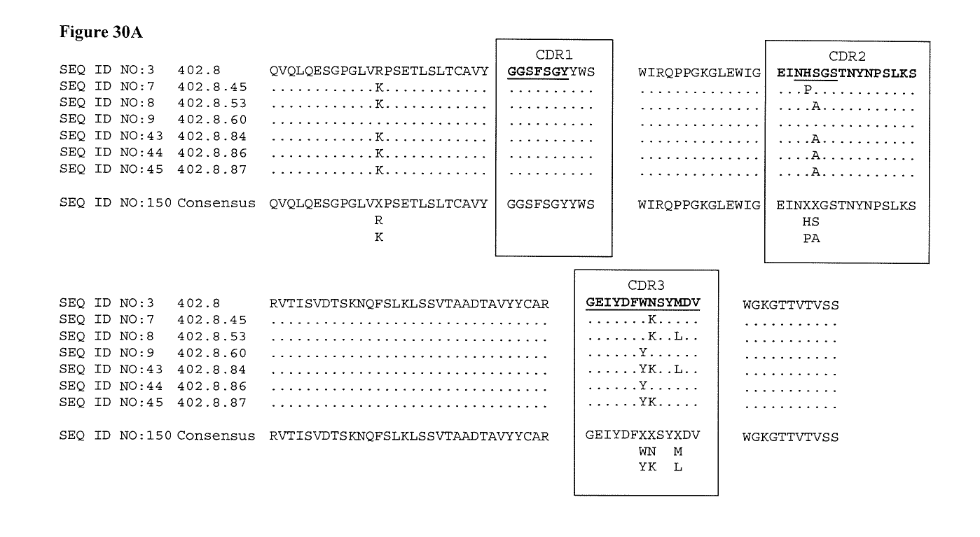

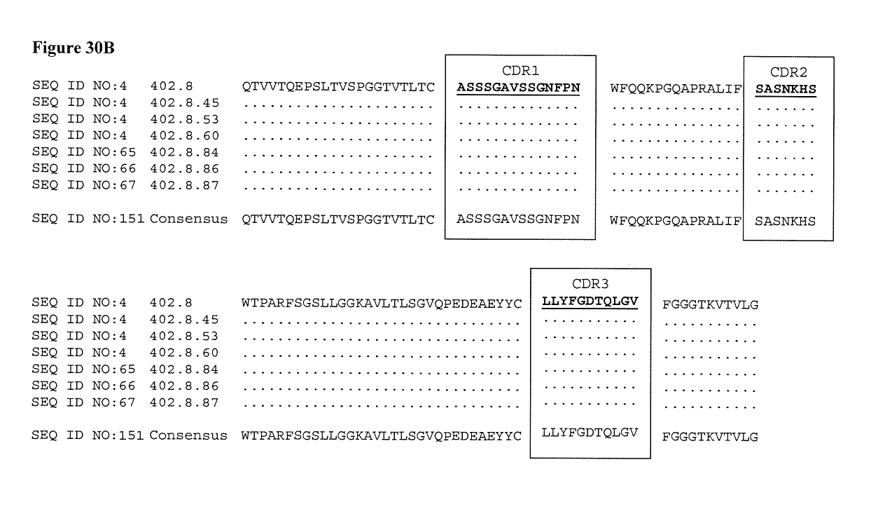

Collectively these regions form a possible binding site, or epitope, to the anti-CD1d antibodies bind to human CD1d. When this regions are analysed on a X-ray crystal structure of human CD1d (such as 3HUJ: PDB database) it can be seen that although these individual regions are distant in terms of the primary amino acid sequence they are located within close proximity to each other in the protein's tertiary structure (FIG. 28A). Both LSYPLE (89-94) and NL (141-142) are located on or about the alpha helices that are present within CD1d and which facilitate the presentation of lipids to the NKT cell receptor. This epitope is located within close proximity to the binding site of the NKT cell receptor β-chain on human CD1d (FIG. 28B). An antibody that binds to this location on CD1d would be capable of competing and inhibiting CD1d-NKT cell receptor β-chain interaction. Such inhibition would prevent formation of the CD1d and the NKT cell receptor complex and thus prevent downstream activation of the NKT cell.

### Example 15

### Ascaris suum Model of Asthma in Cynomolgus Monkeys

The efficacy of anti-CD1d antibodies is examined in a cynomolgus macaque model of asthma. In this protocol, animals sensitized to the nematode parasite Ascaris suum are challenged with aerosolized A. suum extract to induce acute bronchoconstriction followed by pulmonary eosinophilic inflammation and airway hyperactivity in a manner that closely mimics the aetiology of acute asthma exacerbations in humans. Such protocols have been described (Hart, T. et al., (2001) J Allergy Clin Immunol 108:250-257). This model is characterised by many features of chronic human asthma, including mucous cell hyperplasia, subepithelial fibrosis, basement cell membrane thickening, and persistent baseline hyperreactivity to methacholine. Antibody treatment is given prior to challenge with A. suum extract and effect of treatment on airway resistance and compliance, PC50, PCO2 and O2 levels, serum IgE and broncho-alveolar lavage (BAL) measurements, such as IL-4, IL-5 & IL-13 concentrations and relative frequencies of leukocyte subsets such as neutrophils, eosinosphils, mast cells basophils and lymphocytes, can be assessed.

### Example 16

### Alternative Animal Models of Pulmonary Inflammation

### Rhesus Macaque Model of Airway Hyper-Reactivity

The efficacy and safety of anti-CD1d antibodies may be tested in models of airway hyper-reactivity. For example, airway hyper-reactivity can be induced in rhesus macaques (Macaca mulatta) by sequential challenges with house dust mite antigen (from Dermatophagoides farninae). (Seshasayee, D., et al., 20071 Clin Invest 117, 3868-78). Asthma exacerbations, characterised by cough, rapid shallow breathing and increased airway resistance, are induced by challenge with house dust mite extract. Symptoms can be reversed by pharmacological intervention, such as albuterol aerosol treatment. Pulmonary function, e.g. airways resistance and dynamic compliance in response to methacholine challenge, can be measured. Antibody treatment is given prior to re-challenge with house dust mite antigen and pulmonary function as determined by methocholine challenge is assessed.

### Evaluation of Anti-CD1d Antibodies in a Cynomolgus Macaque Model of Airway Hyper-Reactivity Induced by α-GalCer

The efficacy and safety of anti-CD1d antibodies may be tested using a disclosed cynomolgus macaque model of airway hyper-reactivity (Matangkasombut, P., et al., 20081 Allergy Clin Immunol, 121, 1287-9). In this model, cynomolgus macaques that have been sensitized to Ascaris suum are dosed with the specific NKT cell agonist α-galactosylceramide, (α-GalCer), via pulmonary nebuliser. The α-GalCer treatment induces airway hyper-reactivity, as determined by methacholine challenge as described above.

Antibody treatment is given prior to re-challenge with α-GalCer and pulmonary function assessed by methacholine challenge. In addition, bronchoalveolar lavage (BAL) fluid samples is analysed for the presence of mediators associated with airway inflammation, e.g. the concentrations of IL-4, IL-5 and IL-13 in the BAL may be evaluated. Moreover, the cellular infiltrate in the BAL is examined by standard clinical pathology techniques, such as cellular analysis using an automated haematology analyser (for example, Advia systems) to determine the relative frequencies of major leukocyte subsets, such as neutrophils, lymphocytes, eosinophils, mast cells and basophils.

### Example 17

### Alternative Animal Models of Inflammation Driven by NKT Cells

### Ulcerative Colitis

The efficacy and safety of anti-CD1d antibodies are tested in various disclosed models of human inflammatory bowel disease (Wirtz et al., Nat Protoc 2: 541-546, 2007). For example, intrarectal administration of oxazolone induces localized ulceration and inflammation that histologically resembles human ulcerative colitis and is also characterised by diarrhoea, occult blood, weight loss and occasionally rectal prolapse. The pathology of oxazolone-induced colitis may be mediated by NKT cells, particularly via NKT-cell driven secretion of IL-13, and thus disease may be ameliorated by treatment with an anti-CD1d antibody (Heller, F., et al. 2002 Immunity 17, 629-638). In this model, antibody treatment is commenced at the start of colitis induction and effects on weight, stool consistency, occult blood and colon architecture are assessed.

Additionally, anti-CD1d antibodies are tested in a model of murine colitis induced by adoptive transfer of activated (CD4+CD45RBhigh) T cells (Ostanin, D. V., et al (2009) Am J Physiol Gastrointest Liver Physiol 296:G135-G146). In this model, CD4+CD45RBhigh T cells are transferred into immunodeficient mice, resulting in weight loss and diarrhoea, generalised colonic infiltration and inflammation, loss of goblet cells and hyperplasia of colonic epithelium. Antibodies are tested for their ability to reduce weight loss and diarrhoea and to ameliorate colon inflammation and histological changes.

Additionally, anti-CD1d antibodies are tested in a murine model of colitis induced by administration of dextran sulphate sodium (DSS) in drinking water (Wirtz et al., Nat Protoc 2: 541-546, 2007). DSS administration induces robust generalized inflammation of the intestinal tract characterized by erosive lesions and inflammatory infiltrate. Symptoms usually include diarrhoea, occult blood, weight loss and occasionally rectal prolapse. DSS-induced colitis models may be either acute, involving administration of DSS for 7 days, or chronic, involving administration of DSS for longer periods of time.

Antibodies are tested either prophylactically or therapeutically. In a prophylactic model, antibody treatment is commenced at the start of administration of DSS. In a therapeutic model, antibody treatment is commenced several days after commencement of induction. The effect of the treatment on weight and stool consistency, as well as microscopic effects on epithelial integrity and degree of inflammatory infiltrate is determined.

### Non Alcoholic Steatohepatitis

Anti-CD1d antibodies are additionally tested in rodent models of Non-Alcoholic Steatohepatitis (NASH), described for example in Takahashi et al (2012) World Journal of Gastroenterology 18(19): 2300-2308. For example, in C57BL/6 mice, neonatal delivery of streptozotocin (STZ), a hepatotoxic compound, followed by a high-fat diet leads to the development of cardinal features of NASH. As another example, the provision of a high fat diet in C57BL/6 or ob/ob mice can lead to induction and maintenance of NASH. In yet another example, NASH may be generated in rats by a high fat diet plus recurrent and intermittent hypoxemic stress. In one or several of these models, the efficacy of the anti-CD1d antibodies in treated rodents is determined by the effects on total liver weight, serum aminotransferase levels, serum triglyceride levels, blood glucose levels, improvements in liver histopathology and alterations to gene expression patterns.

### Autoimmune Liver Diseases

Anti-CD1d antibodies are additionally tested in animal models of autoimmune liver disease. For example, in mice, hepatitis induced by intravenous injection with concanavalin A (conA) has been described (Takeda, K. et al. (2000) Proc. Natl. Acad. Sci. USA 97(10):5498-5503). ConA is injected into mice through the tail vein. Serum samples are obtained 20 h after Con A injection. Serum aminotransferase [alanine aminotransferase (ALT) and aspartate aminotransferase (AST)] levels are measured using standard photometric techniques. Additionally, liver pathology is assessed by macroscopic and microscopic examination of liver morphology. Antibodies are assessed for effects on serum ALT and AST, and liver histopathology.

### Example 18

### Methods for Generating Binding Proteins

Selection from an Antibody Library Using the 402.8 or 401.11 Antibody

A phage display protocol is used where a first panning round is conducted using an antigen density (i.e., biotinlyated CD1d) of about 100 pmol and a TEA-based elution step as described previously. The second and third rounds use a reduced antigen density (e.g., about 50 pmol). Phage are eluted by adding the 402.8 or 401.11 (or related antibodies) IgG at a 10-fold molar excess and incubating the reactions at room temperature for 2, 5, 10 or 20 mins. The IgG is expected to specifically displace and elute phage expressing FAbs that bound to the 402.8/401.11 epitope. Non-specific binders and phage bound to other regions on the CD1d surface are less likely to elute under these conditions.

The washing regimen comprises six washes with M-PBS for round 1 and 2. For round 3 the washes are three washes with PBST and then three washes with PBS.

Eluted phage are used to infect TG1 E. coli for phagemid rescue or generation of colonies for screening as described for other phage display experiments.

### Selection/Production of Antibodies Using Synthetic CD1d Constructs

Using synthetic CD1d constructs such as hCD1dmu or mCD1dhu as panning reagents for phage display, antibodies that recognize an epitope similar to that of 402.8 and 401.11 may be obtained. A phage display library may be depleted of antibodies that recognize CD1d with a construct like hCD1dmu (SEQ ID NO: 119) (human CD1d in which the key amino acids comprising the epitope are replaced by their corresponding murine amino acid). The library may then be panned against human CD1d. The resultant isolated antibodies will likely bind to amino acids between 89 to 94 and 141 and 142 of human CD1d (SEQ ID NO 116).

### Immunization Approach

Peptide or protein mimics of the 402.8/401.11 antibody epitope on CD1d are used as antigen in place of CD1d in library display technologies or immunization/hybridoma approaches. For example, a chimeric CD1d molecule is constructed to contain the 402.8/401.11 epitope of human CD1d in a framework which is otherwise mouse CD1d (for example mCD1dhu (SEQ ID NO: 118). When such a construct is used as an immunogen in mice, the immune response is expected to be focused towards the non-murine sequence.

### Antibody Sequence ID Concordance

| | | | VL | VL |
|---|---|---|---|---|
| Antibody | VH amino acid | VH nucleotide | amino acid | nucleotide |
| 401.11 | 1 | 10 | 2 | 11 |
| 401.11.24 | 23 | 68 | 46 | 91 |
| 401.11.26 | 24 | 69 | 47 | 92 |
| 401.11.28 | 5 | 14 | 6 | 15 |
| 401.11.86 | 25 | 70 | 48 | 93 |
| 401.11.151 | 26 | 71 | 49 | 94 |
| 401.11.152 | 27 | 72 | 50 | 95 |
| 401.11.154 | 28 | 73 | 51 | 96 |
| 401.11.155 | 29 | 74 | 52 | 97 |
| 401.11.156 | 30 | 75 | 53 | 98 |
| 401.11.157 | 31 | 76 | 54 | 99 |
| 401.11.158 | 32 | 77 | 55 | 100 |
| 401.11.159 | 33 | 78 | 56 | 101 |
| 401.11.160 | 34 | 79 | 57 | 102 |
| 401.11.161 | 35 | 80 | 58 | 103 |
| 401.11.165 | 36 | 81 | 59 | 104 |
| 401.11.166 | 37 | 82 | 60 | 105 |
| 401.11.167 | 38 | 83 | 61 | 106 |
| 401.11.179 | 40 | 85 | 62 | 107 |
| 401.11.180 | 41 | 86 | 63 | 108 |
| 401.11.181 | 42 | 87 | 64 | 109 |
| 402.8 | 3 | 12 | 4 | 13 |
| 402.8.45 | 7 | 16 | 4 | 110 |
| 402.8.53 | 8 | 17 | 4 | 111 |
| 402.8.60 | 9 | 18 | 4 | 112 |
| 402.8.84 | 43 | 88 | 65 | 113 |
| 402.8.86 | 44 | 89 | 66 | 114 |
| 402.8.87 | 45 | 90 | 67 | 115 |

### Other Sequence Descriptions

| SEQ ID NO | Description |
|---|---|
| 19 | human CD1d synthetic construct |
| 20 | human beta-2-microglobulin |
| 21 | TCR alpha chain clone J3N.5 |
| 22 | TCR beta chain clone J3N.5 |
| 117 | murine CD1d extracellular domain construct |
| 118 | mCD1dhu CD1d synthetic construct |
| 119 | hCD1dmu CD1d synthetic construct |
| 120 | Forward primer |
| 121 | Reverse primer |
| 122 | 89-94 of CD1d |
| 123 | 126-142 of CD1d |
| 124 | VH CDR1 (401.11) |
| 125 | VH CDR1 (402.8) |
| 126 | VH CDR3 (401.11) |
| 127 | VH CDR3 (402.8) |
| 128 | VH CDR3 (402.8) |
| 129 | VH CDR3 (402.8) |
| 130 | VH CDR3 (402.8) |
| 131 | VH CDR2 (401.11) |
| 132 | VH CDR2 (402.8) |
| 133 | VH CDR2 (402.8) |
| 134 | VH CDR2 (402.8) |
| 135 | VH CDR1 (401.11) |
| 136 | VH CDR1 (402.8) |
| 137 | VH CDR2 (401.11) |
| 138 | VH CDR2 (402.8) |
| 139 | VH CDR2 (402.8) |
| 140 | VH CDR2 (402.8) |
| 141 | VL CDR1 (401.11) |
| 142 | VL CDR1 (402.8) |
| 143 | VL CDR3 (401.11) |
| 144 | VL CDR3 (402.8) |
| 145 | VL CDR2 (401.11) |
| 146 | VL CDR2 (402.8) |
| 147 | 89-95 of CD1d |
| 148 | 401.11 VH consensus sequence |
| 149 | 401.11VL consensus sequence |
| 150 | 402.8 VH consensus sequence |
| 151 | 402.8 VL consensus sequence |
| 152 | VH CDR3 (401.11) |
| 153 | VH CDR3 (401.11) |
| 154 | VH CDR3 (401.11) |
| 155 | VH CDR3 (402.8) |
| 156 | VH CDR3 (402.8) |
| 157 | Human CD1d |
| 158 | Human heavy chain IgG1 constant domain |
| 159 | Human heavy chain IgG4 constant domain |
| 160 | Human heavy chain IgG4 constant domain (S228P) |
| 161 | Human light chain kappa constant domain |
| 162 | Human light chain lambda constant domain |

We claim:

1. An isolated antibody or antigen binding portion thereof which binds to human CD1d, comprising a V_{H} domain having an amino acid sequence selected from the group consisting of SEQ ID NOs 1, 3, 5, 7, 8, 9, 24, 25, 26, 30, 33, 36, 40, 41, 42, 43, 44 and 45 and sequences at least 95% identical thereto.

2. The isolated antibody or antigen binding portion thereof of claim 1, wherein the amino acid sequence of the VH domain is SEQ ID NO: 148 or SEQ ID NO: 150.

3. An isolated antibody or antigen binding portion thereof which binds to human CD1d, comprising a VL domain having an amino acid sequence selected from the group consisting of SEQ ID NOs 2, 4, 6, 46, 49 and 62 and sequences at least 95% identical thereto.

4. The isolated antibody or antigen binding portion thereof of claim 3, wherein the amino acid sequence of the VL domain is SEQ ID NO: 149 or SEQ ID NO: 4.

5. The isolated antibody or antigen binding portion thereof of claim 1, wherein the amino acid sequence of the VH CDR1 is DYAMH (SEQ ID NO: 124) or GYYWS (SEQ ID NO: 125).

6. The isolated antibody or antigen binding portion thereof of claim 5, wherein the amino acid sequence of the VH CDR3 is DMCSSSGCPDGYFDS (SEQ ID NO: 126), DLCSSGGCPEGYFDS (SEQ ID NO: 152), DMCSSGGCPDGYFDS (SEQ ID NO: 153), DMCSSGGCPEGYFDS (SEQ ID NO: 154), GEIYDFWNSYMDV (SEQ ID NO: 127), GEIYDFWKSYMDV (SEQ ID NO: 128), GEIYDFYKSYLDV (SEQ ID NO: 155), GEIYDFYKSYMDV (SEQ ID NO: 156), GEIYDFWKSYLDV (SEQ ID NO: 129) or GEIYDFYNSYMDV (SEQ ID NO: 130).

7. The isolated antibody or antigen binding portion thereof of claim 5, wherein the amino acid sequence of the VH CDR2 is TIIWNSAIIGYADSVKG (SEQ ID NO: 131), EINHSGSTNYNPSLKS (SEQ ID NO: 132), EINPSGSTNYNPSLKS (SEQ ID NO: 133) or EINHAGSTNYNPSLKS (SEQ ID NO: 134).

8. The isolated antibody or antigen binding portion thereof of claim 1, wherein the amino acid sequence of the VH CDR1 is GFTFDDY (SEQ ID NO: 135) or GGSFSGY (SEQ ID NO: 136).

9. The isolated antibody or antigen binding portion thereof of claim 8, wherein the amino acid sequence of the VH CDR3 is DMCSSSGCPDGYFDS (SEQ ID NO: 126), DLCSSGGCPEGYFDS (SEQ ID NO: 152), DMCSSGGCPDGYFDS (SEQ ID NO: 153), DMCSSGGCPEGYFDS (SEQ ID NO: 154), GEIYDFWNSYMDV (SEQ ID NO: 127), GEIYDFWKSYMDV (SEQ ID NO: 128), GEIYDFYKSYLDV (SEQ ID NO: 155), GEIYDFYKSYMDV (SEQ ID NO: 156), GEIYDFWKSYLDV (SEQ ID NO: 129) or GEIYDFYNSYMDV (SEQ ID NO: 130).

10. The isolated antibody or antigen binding portion thereof of claim 8, wherein the amino acid sequence of the VH CDR2 is IWNSAI (SEQ ID NO: 137), NHSGS (SEQ ID NO: 138), NPSGS (SEQ ID NO: 139) or NHAGS (SEQ ID NO: 140).

11. The isolated antibody or antigen binding portion thereof of claim 3, wherein the amino acid sequence of the VL CDR1 is RASQHISSWLA (SEQ ID NO: 141) or ASSSGAVSSGNFPN (SEQ ID NO: 142).

12. The isolated antibody or antigen binding portion thereof of claim 11, wherein the amino acid sequence of the VL CDR3 is QQANRFPLT (SEQ ID NO: 143) or LLYFGDTQLGV (SEQ ID NO: 144).

13. The isolated antibody or antigen binding portion thereof of claim 11, wherein the amino acid sequence of the VL CDR2 is AASSLQS (SEQ ID NO: 145) or SASNKHS (SEQ ID NO: 146).

14. The isolated antibody or antigen binding portion thereof of claim 1, wherein the isolated antibody or antigen binding portion thereof comprises a VH and VL amino acid sequence pair selected from the group consisting of SEQ ID NO: 1 and SEQ ID NO: 2, SEQ ID NO: 23 and SEQ ID NO: 46, SEQ ID NO: 24 and SEQ ID NO: 47, SEQ ID NO: 5 and SEQ ID NO: 6, SEQ ID NO: 25 and SEQ ID NO: 48, SEQ ID NO: 26 and SEQ ID NO: 49, SEQ ID NO: 27 and SEQ ID NO: 50, SEQ ID NO: 28 and SEQ ID NO: 51, SEQ ID NO: 29 and SEQ ID NO: 52, SEQ ID NO: 30 and SEQ ID NO: 53, SEQ ID NO: 31 and SEQ ID NO: 54, SEQ ID NO: 32 and SEQ ID NO: 55, SEQ ID NO: 33 and SEQ ID NO: 56, SEQ ID NO: 34 and SEQ ID NO: 57, SEQ ID NO: 35 and SEQ ID NO: 58, SEQ ID NO: 36 and SEQ ID NO: 59, SEQ ID NO: 37 and SEQ ID NO: 60, SEQ ID NO: 38 and SEQ ID NO: 61, SEQ ID NO: 40 and SEQ ID NO: 62, SEQ ID NO: 41 and SEQ ID NO: 63, SEQ ID NO: 42 and SEQ ID NO: 64, SEQ ID NO: 3 and SEQ ID NO: 4, SEQ ID NO: 7 and SEQ ID NO: 4, SEQ ID NO: 8 and SEQ ID NO: 4, SEQ ID NO: 9 and SEQ ID NO: 4, SEQ ID NO: 43 and SEQ ID NO: 65, SEQ ID NO: 44 and SEQ ID NO: 66, and SEQ ID NO: 45 and SEQ ID NO: 67.

15. The isolated antibody or antigen binding portion thereof of claim 1, wherein the isolated antibody or antigen binding portion thereof binds to CD1d with an EC₅₀ of from 0.5 ng/ml to 20 ng/ml as measured using a cell based potency assay.

16. The isolated antibody or antigen binding portion thereof of claim 1, wherein the isolated antibody or antigen binding portion thereof comprises a human kappa chain constant region.

17. The isolated antibody or antigen binding portion thereof of claim 3, wherein the isolated antibody or antigen binding portion thereof comprises a human lambda chain constant region.

18. The isolated antibody or antigen binding portion thereof of claim 1, wherein the isolated antibody or antigen binding portion thereof comprises an IgG1 or IgG4 constant region.

19. The isolated antibody or antigen binding portion thereof of claim 18, wherein the isolated antibody or antigen binding portion thereof comprises an IgG4 constant region which includes an S228P mutation.

20. A method of treating a condition involving NKT cell effector function in a human subject, comprising administering to the subject the isolated antibody or antigen binding portion thereof of claim 1, wherein the condition is selected from the group consisting of psoriasis, ulcerative colitis, primary biliary cirrhosis, atherosclerosis, non-alcoholic steatohepatitis, autoimmune hepatitis, ischemia-reperfusion injury, pulmonary inflammation or dysfunction associated with sickle cell disease, and asthma.


SEQUENCE LISTING (187 sequences)

SEQ ID NO: 1 - protein, 124 aa, Homo sapiens
  misc_feature: 401.11 variable heavy chain
QVQLVGSGGGLVKPGRSLRLSCAASGFTFDDYAMHWVRQAPGKGLEWVATIIWNSAIIGYADSVKGRFIVSRDNAKNSLYLQMNSLRAEDMALYYCAKDMCSSSGCPDGYFDSWGQGTLVTVSS

SEQ ID NO: 2 - protein, 108 aa, Homo sapiens
  misc_feature: 401.11 variable light chain
DIQMTQSPSSVSASVGDRVTITCRASQHISSWLAWYQQKPGKAPNLLIYAASSLQSGVSSRFSGSGSGTDFTLTISSLQPEDFATYYCQQANRFPLTFGGGTKLEIKR

SEQ ID NO: 3 - protein, 121 aa, Homo sapiens
  misc_feature: 402.8 variable heavy chain
QVQLQESGPGLVRPSETLSLTCAVYGGSFSGYYWSWIRQPPGKGLEWIGEINHSGSTNYNPSLKSRVTISVDTSKNQFSLKLSSVTAADTAVYYCARGEIYDFWNSYMDVWGKGTTVTVSS

SEQ ID NO: 4 - protein, 112 aa, Homo sapiens
  misc_feature: 402.8 variable light chain
QTVVTQEPSLTVSPGGTVTLTCASSSGAVSSGNFPNWFQQKPGQAPRALIFSASNKHSWTPARFSGSLLGGKAVLTLSGVQPEDEAEYYCLLYFGDTQLGVFGGGTKVTVLG

SEQ ID NO: 5 - protein, 124 aa, Homo sapiens
  misc_feature: 401.11.28 variable heavy chain
QVQLVGSGGGLVKPGRSLRLSCAASGFTFDDYAMHWVRQAPGKGLEWVATIIWNSAIIGYADSVKGRFIVSRDNAKNSLYLQMNSLRAEDTALYYCAKDMCSSSGCPDGYFDSWGQGTLVTVSS

SEQ ID NO: 6 - protein, 108 aa, Homo sapiens
  misc_feature: 401.11.28 variable light chain
DIQMTQSPSSVSASVGDRVTITCRASQHISSWLAWYQQKPGKAPNLLIYAASSLQSGVPSRFSGSGSGTDFTLTISSLQPEDFATYYCQQANRFPLTFGGGTKLEIKR

SEQ ID NO: 7 - protein, 121 aa, Homo sapiens
  misc_feature: 402.8.45 variable heavy chain
QVQLQESGPGLVKPSETLSLTCAVYGGSFSGYYWSWIRQPPGKGLEWIGEINPSGSTNYNPSLKSRVTISVDTSKNQFSLKLSSVTAADTAVYYCARGEIYDFWKSYMDVWGKGTTVTVSS

SEQ ID NO: 8 - protein, 121 aa, Homo sapiens
QVQLQESGPGLVKPSETLSLTCAVYGGSFSGYYWSWIRQPPGKGLEWIGEINHAGSTNYNPSLKSRVTISVDTSKNQFSLKLSSVTAADTAVYYCARGEIYDFWKSYLDVWGKGTTVTVSS

SEQ ID NO: 9 - protein, 121 aa, Homo sapiens
  misc_feature: 402.8.60 variable heavy chain
QVQLQESGPGLVRPSETLSLTCAVYGGSFSGYYWSWIRQPPGKGLEWIGEINHSGSTNYNPSLKSRVTISVDTSKNQFSLKLSSVTAADTAVYYCARGEIYDFYNSYMDVWGKGTTVTVSS

SEQ ID NO: 10 - DNA, 372 bp, Homo sapiens
  misc_feature: 401.11 variable heavy chain
caggtgcagctggtggggtctgggggaggcttggtcaagcctggcaggtccctgagactctcctgtgcagcctctggattcacctttgatgattatgccatgcactgggtccggcaagctccagggaagggcctggagtgggtcgcaactattatttggaatagtgctatcataggctatgcggactctgtgaagggccgattcatcgtctccagagacaacgccaagaactccctgtatctgcaaatgaacagtctgagagcagaggacatggccttgtattactgtgcaaaagatatgtgtagtagtagtggttgccctgatggctactttgactcctggggccagggaaccctggtgacagtgtcctca

SEQ ID NO: 11 - DNA, 324 bp, Homo sapiens
  misc_feature: 401.11 variable light chain
gacatccagatgacccagtctccatcttccgtgtctgcatctgtaggagacagagtcaccatcacttgtcgggcgagtcagcatattagcagctggttagcctggtatcagcagaaaccagggaaagcccctaatctcctgatctatgctgcatccagtttgcaaagtggggtctcatcaaggttcagcggcagtggatctgggacggatttcactctcaccatcagcagcctgcagcctgaagattttgcaacttactattgtcaacaggctaacaggttcccgctcactttcggcggagggaccaagctggagatcaaacgg

SEQ ID NO: 12 - DNA, 363 bp, Homo sapiens
  misc_feature: 402.8 variable heavy chain
caggtgcagctgcaggagtcgggcccaggactggtgaggccttcggagaccctgtccctcacctgcgctgtctatggtgggtccttcagtggttactactggagctggattcgccagcccccagggaaggggctggagtggattggggaaatcaatcatagtggaagcaccaactacaacccgtccctcaagagtcgagtcaccatatcagtagacacgtccaagaaccagttctccctgaagctgagctctgtgaccgccgcggacacggctgtgtattactgtgcgagaggtgagatttacgatttttggaactcctacatggacgtctggggcaaagggaccacggtgacagtgtcctca

SEQ ID NO: 13 - DNA, 336 bp, Homo sapiens
  misc_feature: 402.8 variable light chain
cagactgtggtgactcaggagccctcactgactgtgtccccaggagggacagtcactctcacctgtgcttccagctctggagcagtcagcagtggtaactttccaaactggttccagcagaaacctggacaagcaccccgggcactaatttttagtgcgagcaacaaacattcctggacccctgcccggttttcaggctccctccttgggggcaaagctgtcctgacactgtcaggtgtgcagccagaggacgaggctgaatattactgcctgctctactttggtgatactcaactaggggtgttcggcggagggaccaaggtgaccgtcctaggt

SEQ ID NO: 14 - DNA, 372 bp, Homo sapiens
  misc_feature: 401.11.28 variable heavy chain
caggtgcagctcgtgggctctggcggcggactggtcaagcctggcagaagcctgcggctgagctgtgccgccagcggcttcaccttcgacgactacgccatgcactgggtccgacaggcccctggcaagggcctggaatgggtggccaccatcatctggaacagcgccatcatcggctacgccgacagcgtgaagggccggttcatcgtgtcccgggataacgccaagaacagcctgtacctgcagatgaacagcctgcgggccgaggacaccgccctgtactactgcgccaaggacatgtgcagcagctctggctgccccgacggctacttcgattcttggggccagggcaccctggtcaccgtgtcctca

SEQ ID NO: 15 - DNA, 324 bp, Homo sapiens
  misc_feature: 401.11.28 variable light chain
gacatccagatgacccagagccccagcagcgtgtccgccagcgtgggcgacagagtgaccatcacctgtcgggccagccagcacatcagcagctggctggcctggtatcagcagaagcccggcaaggcccccaacctgctgatctacgccgccagcagcctgcagagcggcgtgccaagcagattcagcggcagcggctccggcaccgacttcaccctgaccatcagctccctgcagcccgaggacttcgccacctactactgccagcaggccaaccggttccccctgacctttggcggaggcaccaagctggaaatcaagcgt

SEQ ID NO: 16 - DNA, 363 bp, Homo sapiens
  misc_feature: 402.8.45 variable heavy chain
caggtgcagctgcaggagtcgggcccaggactggtgaagccttcggagaccctgtccctcacctgcgctgtctatggtgggtccttcagtggttactactggagctggattcgccagcccccagggaaggggctggagtggattggggaaatcaatcccagtggaagcaccaactacaacccgtccctcaagagtcgagtcaccatatcagtagacacgtccaagaaccagttctccctgaagctgagctctgtgaccgccgcggacacggctgtgtattactgtgcgagaggtgagatttacgatttttggaagtcctacatggacgtctggggcaaagggaccacggtgacagtgtcctca

SEQ ID NO: 17 - DNA, 362 bp, Homo sapiens
  misc_feature: 402.8.53 variable heavy chain
caggtgcagctgcaggagtcgggcccaggactggtgaagccttcggagaccctgtccctcacctgcgctgtctatggtgggtccttcagtggttactactggagctggattcgccagcccccagggaaggggctggagtggattggggaaatcaatcatgccggaagcaccaactacaacccgtccctcaagagtcgagtcaccatatcagtagacacgtccaagaaccagttctccctgaagctgagctctgtgaccgccgcggacacggctgtgtattactgtgcgagaggtgagatttacgatttttggaagtcctacctggacgtctggggcaaagggaccacggtgacaggtcctca

SEQ ID NO: 18 - DNA, 362 bp, Homo sapiens
  misc_feature: 402.8.60 variable heavy chain
caggtgcagctgcaggagtcgggcccaggactggtgaggccttcggagaccctgtccctcacctgcgctgtctatggtgggtccttcagtggttactactggagctggattcgccagccccagggaaggggctggagtggattggggaaatcaatcatagtggaagcaccaactacaacccgtccctcaagagtcgagtcaccatatcagtagacacgtccaagaaccagttctccctgaagctgagctctgtgaccgccgcggacacggctgtgtattactgtgcgagaggtgagatttacgatttttacaactcctacatggacgtctggggcaaagggaccacggtgacagtgtcctca

SEQ ID NO: 19 - protein, 322 aa, Homo sapiens
  misc_feature: human CD1d synthetic construct
MGCLLFLLLWALLQAWGSAEVPQRLFPLRCLQISSFANSSWTRTDGLAWLGELQTHSWSNDSDTVRSLKPWSQGTFSDQQWETLQHIFRVYRSSFTRDVKEFAKMLRLSYPLELQVSAGCEVHPGNASNNFFHVAFQGKDILSFQGTSWEPTQEAPLWVNLAIQVLNQDKWTRETVQWLLNGTCPQFVSGLLESGKSELKKQVKPKAWLSRGPSPGPGRLLLVCHVSGFYPKPVWVKWMRGEQEQQGTQPGDILPNADETWYLRATLDVVAGEAAGLSCRVKHSSLEGQDIVLYWGGSYTSGSLVPRGSGSKRVHHHHHHHH

SEQ ID NO: 20 - protein, 119 aa, Homo sapiens
  misc_feature: human beta-2-microglobulin
MSRSVALAVLALLSLSGLEAIQRTPKIQVYSRHPAENGKSNFLNCYVSGFHPSDIEVDLLKNGERIEKVEHSDLSFSKDWSFYLLYYTEFTPTEKDEYACRVNHVTLSQPKIVKWDRDM

SEQ ID NO: 21 - protein, 276 aa, Homo sapiens
  misc_feature: TCR alpha chain clone J3N.5
MKKHLTTFLVILWLYFYRGNGKNQVEQSPQSLIILEGKNCTLQCNYTVSPFSNLRWYKQDTGRGPVSLTIMTFSENTKSNGRYTATLDADTKQSSLHITASQLSDSASYICVVSDRGSTLGRLYFGRGTQLTVWPDIQNPDPAVYQLRDSKSSDKSVCLFTDFDSQTNVSQSKDSDVYITDKTVLDMRSMDFKSNSAVAWSNKSDFACANAFNNSIIPEDTFFPSPESSCDVKLVEKSFETDTNLNFQNLSVIGFRILLLKVAGFNLLMTLRLWSS

SEQ ID NO: 22 - protein, 315 aa, Homo sapiens [sequence omitted]

SEQ ID NO: 23 - protein, 124 aa, Homo sapiens; identical to SEQ ID NO: 5
  misc_feature: 401.11.24 variable heavy chain protein sequence

SEQ ID NO: 24 - protein, 124 aa, Homo sapiens
  misc_feature: 401.11.26 variable heavy chain protein sequence
EVQLVESGGGLVQPGRSLRLSCAASGFTFDDYAMHWVRQAPGKGLEWVATIIWNSAIIGYADSVKGRFIVSRDNAKNSLYLQMNSLRAEDMALYYCAKDMCSSSGCPDGYFDSWGQGTLVTVSS

SEQ ID NO: 25 - protein, 124 aa, Homo sapiens
  misc_feature: 401.11.86 variable heavy chain protein sequence
QVQLVGSGGGLVKPGRSLRLSCAASGFTFDDYAMHWVRQAPGKGLEWVATIIWNSAIIGYADSVKGRFIVSRDNAKNSLYLQMNSLRAEDMALYYCAKDMCSSGGCPDGYFDSWGQGTLVTVSS

SEQ ID NO: 26 - protein, 124 aa, Homo sapiens
  misc_feature: 401.11.151 variable heavy chain protein sequence
EVQLVESGGGLVQPGRSLRLSCAASGFTFDDYAMHWVRQAPGKGLEWVATIIWNSAIIGYADSVKGRFIVSRDNAKNSLYLQMNSLRAEDTALYYCAKDMCSSSGCPDGYFDSWGQGTLVTVSS

SEQ ID NO: 27 - protein, 124 aa, Homo sapiens; identical to SEQ ID NO: 26
  misc_feature: 401.11.152 variable heavy chain protein sequence

SEQ ID NO: 28 - protein, 124 aa, Homo sapiens; identical to SEQ ID NO: 5
  misc_feature: 401.11.154 variable heavy chain protein sequence

SEQ ID NO: 29 - protein, 124 aa, Homo sapiens; identical to SEQ ID NO: 24
  misc_feature: 401.11.155 variable heavy chain protein sequence

SEQ ID NO: 30 - protein, 124 aa, Homo sapiens
  misc_feature: 401.11.156 variable heavy chain protein sequence
QVQLVGSGGGLVKPGRSLRLSCAASGFTFDDYAMHWVRQAPGKGLEWVATIIWNSAIIGYADSVKGRFIVSRDNAKNSLYLQMNSLRAEDTALYYCAKDMCSSGGCPDGYFDSWGQGTLVTVSS

SEQ ID NO: 31 - protein, 124 aa, Homo sapiens; identical to SEQ ID NO: 30
  misc_feature: 401.11.157 variable heavy chain protein sequence

SEQ ID NO: 32 - protein, 124 aa, Homo sapiens; identical to SEQ ID NO: 30
  misc_feature: 401.11.158 variable heavy chain protein sequence

SEQ ID NO: 33 - protein, 124 aa, Homo sapiens;
  misc_feature: 401.11.159 variable heavy chain protein sequence
EVQLVESGGGLVQPGRSLRLSCAASGFTFDDYAMHWVRQAPGKGLEWVATIIWNSAIIGYADSVKGRFIVSRDNAKNSLYLQMNSLRAEDMALYYCAKDMCSSGGCPDGYFDSWGQGTLVTVSS

SEQ ID NO: 34 - protein, 124 aa, Homo sapiens;; identical to SEQ ID NO: 33
  misc_feature: 401.11.160 variable heavy chain protein sequence

SEQ ID NO: 35 - protein, 124 aa, Homo sapiens; identical to SEQ ID NO: 33
  misc_feature: 401.11.161 variable heavy chain protein sequence

SEQ ID NO: 36 - protein, 124 aa, Homo sapiens
  misc_feature: 401.11.165 variable heavy chain protein sequence
EVQLVESGGGLVQPGRSLRLSCAASGFTFDDYAMHWVRQAPGKGLEWVATIIWNSAIIGYADSVKGRFIVSRDNAKNSLYLQMNSLRAEDTALYYCAKDMCSSGGCPDGYFDSWGQGTLVTVSS

SEQ ID NO: 37 - protein, 124 aa, Homo sapiens; identical to SEQ ID NO: 36
  misc_feature: 401.11.166 variable heavy chain protein sequence

SEQ ID NO: 38 - protein, 124 aa, Homo sapiens; identical to SEQ ID NO: 36
  misc_feature: 401.11.167 variable heavy chain protein sequence

SEQ ID NO: 39 - protein, 124 aa, Homo sapiens; identical to SEQ ID NO: 30
  misc_feature: 401.11.158 variable heavy chain protein sequence

SEQ ID NO: 40 - protein, 124 aa, Homo sapiens
  misc_feature: 401.11.179 variable heavy chain protein sequence
QVQLVGSGGGLVKPGRSLRLSCAASGFTFDDYAMHWVRQAPGKGLEWVATIIWNSAIIGYADSVKGRFIVSRDNAKNSLYLQMNSLRAEDTALYYCAKDMCSSGGCPEGYFDSWGQGTLVTVSS

SEQ ID NO: 41 - protein, 124 aa, Homo sapiens
  misc_feature: 401.11.180 variable heavy chain protein sequence
QVQLVGSGGGLVKPGRSLRLSCAASGFTFDDYAMHWVRQAPGKGLEWVATIIWNSAIIGYADSVKGRFIVSRDNAKNSLYLQMNSLRAEDTALYYCAKDLCSSGGCPEGYFDSWGQGTLVTVSS

SEQ ID NO: 42 - protein, 124 aa, Homo sapiens
  misc_feature: 401.11.181 variable heavy chain protein sequence
QVQLVESGGGLVKPGRSLRLSCAASGFTFDDYAMHWVRQAPGKGLEWVATIIWNSAIIGYADSVKGRFIVSRDNAKNSLYLQMNSLRAEDTALYYCAKDMCSSGGCPDGYFDSWGQGTLVTVSS

SEQ ID NO: 43 - protein, 121 aa, Homo sapiens
  misc_feature: 402.8.84 variable heavy chain protein sequence
QVQLQESGPGLVKPSETLSLTCAVYGGSFSGYYWSWIRQPPGKGLEWIGEINHAGSTNYNPSLKSRVTISVDTSKNQFSLKLSSVTAADTAVYYCARGEIYDFYKSYLDVWGKGTTVTVSS

SEQ ID NO: 44 - protein, 121 aa, Homo sapiens
  misc_feature: 402.8.86 variable heavy chain protein sequence
QVQLQESGPGLVKPSETLSLTCAVYGGSFSGYYWSWIRQPPGKGLEWIGEINHAGSTNYNPSLKSRVTISVDTSKNQFSLKLSSVTAADTAVYYCARGEIYDFYNSYMDVWGKGTTVTVSS

SEQ ID NO: 45 - protein, 121 aa, Homo sapiens
  misc_feature: 402.8.87 variable heavy chain protein sequence
QVQLQESGPGLVKPSETLSLTCAVYGGSFSGYYWSWIRQPPGKGLEWIGEINHAGSTNYNPSLKSRVTISVDTSKNQFSLKLSSVTAADTAVYYCARGEIYDFYKSYMDVWGKGTTVTVSS

SEQ ID NO: 46 - protein, 108 aa, Homo sapiens
  misc_feature: 401.11.24 variable light chain protein sequence
DIQMTQSPSSVSASVGDRVTITCRASQHISSWLAWYQQKPGKAPKLLIYAASSLQSGVSSRFSGSGSGTDFTLTISSLQPEDFATYYCQQANRFPLTFGGGTKLEIKR

SEQ ID NO: 47 - protein, 108 aa, Homo sapiens; identical to SEQ ID NO: 6
  misc_feature: 401.11.26 variable light chain protein sequence

SEQ ID NO: 48 - protein, 108 aa, Homo sapiens; identical to SEQ ID NO: 2
  misc_feature: 401.11.86 variable light chain protein sequence

SEQ ID NO: 49 - protein, 108 aa, Homo sapiens
  misc_feature: 401.11.151 variable light chain protein sequence
DIQMTQSPSSVSASVGDRVTITCRASQHISSWLAWYQQKPGKAPKLLIYAASSLQSGVPSRFSGSGSGTDFTLTISSLQPEDFATYYCQQANRFPLTFGGGTKLEIKR

SEQ ID NO: 50 - protein, 108 aa, Homo sapiens; identical to SEQ ID NO: 46
  misc_feature: 401.11.152 variable light chain protein sequence

SEQ ID NO: 51 - protein, 108 aa, Homo sapiens; identical to SEQ ID NO: 49
  misc_feature: 401.11.154 variable light chain protein sequence

SEQ ID NO: 52 - protein, 108 aa, Homo sapiens; identical to SEQ ID NO: 49
  misc_feature: 401.11.155 variable light chain protein sequence

SEQ ID NO: 53 - protein, 108 aa, Homo sapiens; identical to SEQ ID NO: 46
  misc_feature: 401.11.156 variable light chain protein sequence

SEQ ID NO: 54 - protein, 108 aa, Homo sapiens; identical to SEQ ID NO: 6
  misc_feature: 401.11.157 variable light chain protein sequence

SEQ ID NO: 55 - protein, 108 aa, Homo sapiens; identical to SEQ ID NO: 49
  misc_feature: 401.11.158 variable light chain protein sequence

SEQ ID NO: 56 - protein, 108 aa, Homo sapiens; identical to SEQ ID NO: 46
  misc_feature: 401.11.159 variable light chain protein sequence

SEQ ID NO: 57 - protein, 108 aa, Homo sapiens; identical to SEQ ID NO: 6
  misc_feature: 401.11.160 variable light chain protein sequence

SEQ ID NO: 58 - protein, 108 aa, Homo sapiens; identical to SEQ ID NO: 49
  misc_feature: 401.11.161 variable light chain protein sequence

SEQ ID NO: 59 - protein, 108 aa, Homo sapiens; identical to SEQ ID NO: 49
  misc_feature: 401.11.165 variable light chain protein sequence

SEQ ID NO: 60 - protein, 108 aa, Homo sapiens; identical to SEQ ID NO: 46
  misc_feature: 401.11.166 variable light chain protein sequence

SEQ ID NO: 61 - protein, 108 aa, Homo sapiens; identical to SEQ ID NO: 6
  misc_feature: 401.11.167 variable light chain protein sequence

SEQ ID NO: 62 - protein, 108 aa, Homo sapiens; identical to SEQ ID NO: 49
  misc_feature: 401.11.179 variable light chain protein sequence

SEQ ID NO: 63 - protein, 108 aa, Homo sapiens; identical to SEQ ID NO: 49
  misc_feature: 401.11.180 variable light chain protein sequence

SEQ ID NO: 64 - protein, 108 aa, Homo sapiens; identical to SEQ ID NO: 49
  misc_feature: 401.11.181 variable light chain protein sequence

SEQ ID NO: 65 - protein, 112 aa, Homo sapiens; identical to SEQ ID NO: 4
  misc_feature: 402.8.84 variable light chain protein sequence

SEQ ID NO: 66 - protein, 112 aa, Homo sapiens; identical to SEQ ID NO: 4
  misc_feature: 402.8.86 variable light chain protein sequence

SEQ ID NO: 67 - protein, 112 aa, Homo sapiens; identical to SEQ ID NO: 4
  misc_feature: 402.8.87 variable light chain protein sequence

SEQ ID NO: 68 - DNA, 372 bp, Homo sapiens; identical to SEQ ID NO: 14
  misc_feature: 401.11.24 variable heavy chain nucleotide

SEQ ID NO: 69 - DNA, 372 bp, Homo sapiens
  misc_feature: 401.11.26 variable heavy chain nucleotide
gaggtgcagctggtggaatctggcggcggactggtgcagcccggcagaagcctgagactgagctgtgccgccagcggcttcaccttcgacgactacgccatgcactgggtccgacaggcccctggcaagggcctggaatgggtggccaccatcatctggaacagcgccatcatcggctacgccgacagcgtgaagggccggttcatcgtgtcccgggataacgccaagaacagcctgtacctgcagatgaacagcctgcgggccgaggacatggccctgtactactgcgccaaggacatgtgcagcagcagcggctgccccgacggctacttcgattcttggggccagggcaccctggtcaccgtgtcctca

SEQ ID NO: 70 - DNA, 372 bp, Homo sapiens
  misc_feature: 401.11.86 variable heavy chain nucleotide
caggtgcagctcgtgggctctggcggcggactggtcaagcctggcagaagcctgcggctgagctgtgccgccagcggcttcaccttcgacgactacgccatgcactgggtccgacaggcccctggcaagggcctggaatgggtggccaccatcatctggaacagcgccatcatcggctacgccgacagcgtgaagggccggttcatcgtgtcccgggataacgccaagaacagcctgtacctgcagatgaacagcctgcgggccgaggacatggccctgtactactgcgccaaggacatgtgcagcagcggcggctgccccgacggctacttcgattcttggggccagggcaccctggtcaccgtgtcctca

SEQ ID NO: 71 - DNA, 372 bp, Homo sapiens
  misc_feature: 401.11.151 variable heavy chain nucleotide
gaggtgcagctggtggaatctggcggcggactggtgcagcccggcagaagcctgagactgagctgtgccgccagcggcttcaccttcgacgactacgccatgcactgggtccgacaggcccctggcaagggcctggaatgggtggccaccatcatctggaacagcgccatcatcggctacgccgacagcgtgaagggccggttcatcgtgtcccgggataacgccaagaacagcctgtacctgcagatgaacagcctgcgggccgaggacaccgccctgtactactgcgccaaggacatgtgcagcagcagcggctgccccgacggctacttcgattcttggggccagggcaccctggtcaccgtgtcctca

SEQ ID NO: 72 - DNA, 372 bp, Homo sapiens; identical to SEQ ID NO: 71
  misc_feature: 401.11.152 variable heavy chain nucleotide

SEQ ID NO: 73 - DNA, 372 bp, Homo sapiens; identical to SEQ ID NO: 14
  misc_feature: 401.11.154 variable heavy chain nucleotide

SEQ ID NO: 74 - DNA, 372 bp, Homo sapiens; identical to SEQ ID NO: 69
  misc_feature: 401.11.155 variable heavy nucleotide

SEQ ID NO: 75 - DNA, 372 bp, Homo sapiens
  misc_feature: 401.11.156 variable heavy chain nucleotide
caggtgcagctcgtgggctctggcggcggactggtcaagcctggcagaagcctgcggctgagctgtgccgccagcggcttcaccttcgacgactacgccatgcactgggtccgacaggcccctggcaagggcctggaatgggtggccaccatcatctggaacagcgccatcatcggctacgccgacagcgtgaagggccggttcatcgtgtcccgggataacgccaagaacagcctgtacctgcagatgaacagcctgcgggccgaggacaccgccctgtactactgcgccaaggacatgtgcagcagcggtggctgccccgacggctacttcgattcttggggccagggcaccctggtcaccgtgtcctca

SEQ ID NO: 76 - DNA, 372 bp, Homo sapiens; identical to SEQ ID NO: 75
  misc_feature: 401.11.157 variable heavy chain nucleotide

SEQ ID NO: 77 - DNA, 372 bp, Homo sapiens; identical to SEQ ID NO: 75
  misc_feature: 401.11.158 variable heavy chain nucleotide

SEQ ID NO: 78 - DNA, 372 bp, Homo sapiens
  misc_feature: 401.11.159 variable heavy chain nucleotide
gaggtgcagctggtggaatctggcggcggactggtgcagcccggcagaagcctgagactgagctgtgccgccagcggcttcaccttcgacgactacgccatgcactgggtccgacaggcccctggcaagggcctggaatgggtggccaccatcatctggaacagcgccatcatcggctacgccgacagcgtgaagggccggttcatcgtgtcccgggataacgccaagaacagcctgtacctgcagatgaacagcctgcgggccgaggacatggccctgtactactgcgccaaggacatgtgcagcagcggtggctgccccgacggctacttcgattcttggggccagggcaccctggtcaccgtgtcctca

SEQ ID NO: 79 - DNA, 372 bp, Homo sapiens; identical to SEQ ID NO: 78
  misc_feature: 401.11.160 variable heavy chain nucleotide

SEQ ID NO: 80 - DNA, 372 bp, Homo sapiens; identical to SEQ ID NO: 78
  misc_feature: 401.11.161 variable heavy chain nucleotide

SEQ ID NO: 81 - DNA, 372 bp, Homo sapiens;
  misc_feature: 401.11.165 variable heavy chain nucleotide
gaggtgcagctggtggaatctggcggcggactggtgcagcccggcagaagcctgagactgagctgtgccgccagcggcttcaccttcgacgactacgccatgcactgggtccgacaggcccctggcaagggcctggaatgggtggccaccatcatctggaacagcgccatcatcggctacgccgacagcgtgaagggccggttcatcgtgtcccgggataacgccaagaacagcctgtacctgcagatgaacagcctgcgggccgaggacaccgccctgtactactgcgccaaggacatgtgcagcagcggtggctgccccgacggctacttcgattcttggggccagggcaccctggtcaccgtgtcctca

SEQ ID NO: 82 - DNA, 372 bp, Homo sapiens; identical to SEQ ID NO: 81
  misc_feature: 401.11.166 variable heavy chain nucleotide

SEQ ID NO: 83 - DNA, 372 bp, Homo sapiens; identical to SEQ ID NO: 81
  misc_feature: 401.11.167 variable heavy chain nucleotide

SEQ ID NO: 84 - DNA, 372 bp, Homo sapiens
  misc_feature: 401.11.158 variable heavy chain nucleotide
caggtgcagctggtgggctctggcggcggactggtgaaacccggcagaagcctgagactgagctgcgccgccagcggcttcaccttcgacgactacgccatgcactgggtccgccaggcccctggcaagggactggaatgggtggcaaccatcatctggaacagcgccatcatcggctacgccgacagcgtgaagggccggttcatcgtgtccagagacaacgccaagaacagcctgtacctgcagatgaacagcctgcgggccgaggacaccgccctgtactactgcgccaaggacatgtgcagcagcgggggctgccccgacggctactttgatagctggggccagggcaccctggtgacagtgtcctca

SEQ ID NO: 85 - DNA, 372 bp, Homo sapiens
  misc_feature: 401.11.179 variable heavy chain nucleotide
caggtgcagctggtgggctctggcggcggactggtgaaacccggcagaagcctgagactgagctgcgccgccagcggcttcaccttcgacgactacgccatgcactgggtccgccaggcccctggcaagggactggaatgggtggcaaccatcatctggaacagcgccatcatcggctacgccgacagcgtgaagggccggttcatcgtgtccagagacaacgccaagaacagcctgtacctgcagatgaacagcctgcgggccgaggacaccgccctgtactactgcgccaaggacatgtgcagcagcgggggctgccccgaaggctactttgatagctggggccagggcaccctggtgacagtgtcctca

SEQ ID NO: 86 - DNA, 372 bp, Homo sapiens
  misc_feature: 401.11.180 variable heavy chain nucleotide
caggtgcagctggtgggctctggcggcggactggtgaaacccggcagaagcctgagactgagctgcgccgccagcggcttcaccttcgacgactacgccatgcactgggtccgccaggcccctggcaagggactggaatgggtggcaaccatcatctggaacagcgccatcatcggctacgccgacagcgtgaagggccggttcatcgtgtccagagacaacgccaagaacagcctgtacctgcagatgaacagcctgcgggccgaggacaccgccctgtactactgcgccaaggacctgtgcagcagcgggggctgccccgaaggctactttgatagctggggccagggcaccctggtgacagtgtcctca

SEQ ID NO: 87 - DNA, 372 bp, Homo sapiens
  misc_feature: 401.11.181 variable heavy chain nucleotide
caggtgcagctggtggaatctggcggcggactggtgaaacccggcagaagcctgagactgagctgcgccgccagcggcttcaccttcgacgactacgccatgcactgggtccgccaggcccctggcaagggactggaatgggtggcaaccatcatctggaacagcgccatcatcggctacgccgacagcgtgaagggccggttcatcgtgtccagagacaacgccaagaacagcctgtacctgcagatgaacagcctgcgggccgaggacaccgccctgtactactgcgccaaggacatgtgcagcagcgggggctgccccgacggctactttgatagctggggccagggcaccctggtgacagtgtcctca

SEQ ID NO: 88 - DNA, 363 bp, Homo sapiens
  misc_feature: 402.8.84 variable heavy chain nucleotide
caggtgcagctgcaggagtcgggcccaggactggtgaagccttcggagaccctgtccctcacctgcgctgtctatggtgggtccttcagtggttactactggagctggattcgccagcccccagggaaggggctggagtggattggggaaatcaatcatgccggaagcaccaactacaacccgtccctcaagagtcgagtcaccatatcagtagacacgtccaagaaccagttctccctgaagctgagctctgtgaccgccgcggacacggctgtgtattactgtgcgagaggtgagatttacgatttttacaagtcctacctggacgtctggggcaaagggaccacggtgacagtgtcctca

SEQ ID NO: 89 - DNA, 363 bp, Homo sapiens
  misc_feature: 402.8.86 variable heavy chain nucleotide
caggtgcagctgcaggagtcgggcccaggactggtgaagccttcggagaccctgtccctcacctgcgctgtctatggtgggtccttcagtggttactactggagctggattcgccagcccccagggaaggggctggagtggattggggaaatcaatcatgccggaagcaccaactacaacccgtccctcaagagtcgagtcaccatatcagtagacacgtccaagaaccagttctccctgaagctgagctctgtgaccgccgcggacacggctgtgtattactgtgcgagaggtgagatttacgatttttacaactcctacatggacgtctggggcaaagggaccacggtgacagtgtcctca

SEQ ID NO: 90 - DNA, 363 bp, Homo sapiens
  misc_feature: 402.8.87 variable heavy chain nucleotide
caggtgcagctgcaggagtcgggcccaggactggtgaagccttcggagaccctgtccctcacctgcgctgtctatggtgggtccttcagtggttactactggagctggattcgccagcccccagggaaggggctggagtggattggggaaatcaatcatgccggaagcaccaactacaacccgtccctcaagagtcgagtcaccatatcagtagacacgtccaagaaccagttctccctgaagctgagctctgtgaccgccgcggacacggctgtgtattactgtgcgagaggtgagatttacgatttttacaagtcctacatggacgtctggggcaaagggaccacggtgacagtgtcctca

SEQ ID NO: 91 - DNA, 324 bp, Homo sapiens
  misc_feature: 401.11.24 variable light chain nucleotide
gacatccagatgacccagagccccagcagcgtgtccgccagcgtgggcgacagagtgaccatcacctgtcgggccagccagcacatcagcagctggctggcctggtatcagcagaagcccggcaaggcccccaagctgctgatctacgccgccagcagcctgcagagcggcgtgtccagcagattcagcggcagcggctccggcaccgacttcaccctgaccatcagctccctgcagcccgaggacttcgccacctactactgccagcaggccaaccggttccccctgacctttggcggaggcaccaagctggaaatcaagcgt

SEQ ID NO: 92 - DNA, 324 bp, Homo sapiens; identical to SEQ ID NO: 15
  misc_feature: 401.11.26 variable light chain nucleotide

SEQ ID NO: 93 - DNA, 324 bp, Homo sapiens
  misc_feature: 401.11.86 variable light chain nucleotide
gacatccagatgacccagtctccatcttccgtgtctgcatctgtaggagacagagtcaccatcacttgtcgggcgagtcagcatattagcagctggttagcctggtatcagcagaaaccagggaaagcccctaatctcctgatctatgctgcatccagtttgcaaagtggggtctcatcaaggttcagcggcagtggatctgggacggatttcactctcaccatcagcagcctgcagcctgaagattttgcaacttactattgtcaacaggctaacaggttcccgctcactttcggcggagggaccaagctggagatcaaacgt

SEQ ID NO: 94 - DNA, 324 bp, Homo sapiens
  misc_feature: 401.11.151 variable light chain nucleotide
gacatccagatgacccagagccccagcagcgtgtccgccagcgtgggcgacagagtgaccatcacctgtcgggccagccagcacatcagcagctggctggcctggtatcagcagaagcccggcaaggcccccaagctgctgatctacgccgccagcagcctgcagagcggcgtgcccagcagattcagcggcagcggctccggcaccgacttcaccctgaccatcagctccctgcagcccgaggacttcgccacctactactgccagcaggccaaccggttccccctgacctttggcggaggcaccaagctggaaatcaagcgt

SEQ ID NO: 95 - DNA, 324 bp, Homo sapiens; identical to SEQ ID NO: 91
  misc_feature: 401.11.152 variable light chain nucleotide

SEQ ID NO: 96 - DNA, 324 bp, Homo sapiens; identical to SEQ ID NO: 94
  misc_feature: 401.11.154 variable light chain nucleotide

SEQ ID NO: 97 - DNA, 324 bp, Homo sapiens; identical to SEQ ID NO: 94
  misc_feature: 401.11.155 variable light chain nucleotide

SEQ ID NO: 98 - DNA, 324 bp, Homo sapiens;; identical to SEQ ID NO: 91
  misc_feature: 401.11.156 variable light chain nucleotide

SEQ ID NO: 99 - DNA, 324 bp, Homo sapiens; identical to SEQ ID NO: 15
  misc_feature: 401.11.157 variable light chain nucleotide

SEQ ID NO: 100 - DNA, 324 bp, Homo sapiens; identical to SEQ ID NO: 94
  misc_feature: 401.11.158 variable light chain nucleotide

SEQ ID NO: 101 - DNA, 324 bp, Homo sapiens; identical to SEQ ID NO: 91
  misc_feature: 401.11.159 variable light chain nucleotide

SEQ ID NO: 102 - DNA, 324 bp, Homo sapiens; identical to SEQ ID NO: 15
  misc_feature: 401.11.160 variable light chain nucleotide

SEQ ID NO: 103 - DNA, 324 bp, Homo sapiens; identical to SEQ ID NO: 94
  misc_feature: 401.11.161 variable light chain nucleotide

SEQ ID NO: 104 - DNA, 324 bp, Homo sapiens; identical to SEQ ID NO: 94
  misc_feature: 401.11.165 variable light chain nucleotide

SEQ ID NO: 105 - DNA, 324 bp, Homo sapiens; identical to SEQ ID NO: 91
  misc_feature: 401.11.166 variable light chain nucleotide

SEQ ID NO: 106 - DNA, 324 bp, Homo sapiens; identical to SEQ ID NO: 15
  misc_feature: 401.11.167 variable light chain nucleotide

SEQ ID NO: 107 - DNA, 324 bp, Homo sapiens
  misc_feature: 401.11.179 variable light chain nucleotide
gacatccagatgacccagagccccagcagcgtgtccgccagcgtgggcgacagagtgaccatcacctgtcgggccagccagcacatcagcagctggctggcctggtatcagcagaagcccggcaaggcccccaagctcctgatctacgccgccagctctctgcagtctggcgtgccaagcagattcagcggcagcggctccggcaccgacttcaccctgaccatcagctccctgcagcccgaggacttcgccacctactactgccagcaggccaaccggttccccctgaccttcggcggaggcaccaagctggaaatcaagcgg

SEQ ID NO: 108 - DNA, 324 bp, Homo sapiens; identical to SEQ ID NO: 107
  misc_feature: 401.11.180 variable light chain nucleotide

SEQ ID NO: 109 - DNA, 324 bp, Homo sapiens; identical to SEQ ID NO: 107
  misc_feature: 401.11.181 variable light chain nucleotide

SEQ ID NO: 110 - DNA, 336 bp, Homo sapiens
  misc_feature: 402.8.45 variable light chain nucleotide
cagactgtggtgactcaggagccctcactgactgtgtccccaggagggacagtcactctcacctgtgcttccagctctggagcagtcagcagtggtaactttccaaactggttccagcagaaacctggacaagcaccccgggcactaatttttagtgcgagcaacaaacattcctggacccctgcccggttttcaggctccctccttgggggcaaagctgtcctgacactgtcaggtgtgcagcctgaggacgaggctgaatattactgcctgctctactttggtgatactcaactaggggtgttcggcggagggaccaaggtgaccgtcctaggt

SEQ ID NO: 111 - DNA, 336 bp, Homo sapiens; identical to SEQ ID NO: 110
  misc_feature: 402.8.53 variable light chain nucleotide

SEQ ID NO: 112 - DNA, 336 bp, Homo sapiens; identical to SEQ ID NO: 110
  misc_feature: 402.8.60 variable light chain nucleotide

SEQ ID NO: 113 - DNA, 336 bp, Homo sapiens; identical to SEQ ID NO: 110
  misc_feature: 402.8.84 variable light chain nucleotide

SEQ ID NO: 114 - DNA, 336 bp, Homo sapiens; identical to SEQ ID NO: 110
  misc_feature: 402.8.86 variable light chain nucleotide

SEQ ID NO: 115 - DNA, 336 bp, Homo sapiens; identical to SEQ ID NO: 110
  misc_feature: 402.8.87 variable light chain nucleotide

SEQ ID NO: 116 - protein, 303 aa, Homo sapiens
  misc_feature: human CD1d extracellular domain construct
EVPQRLFPLRCLQISSFANSSWTRTDGLAWLGELQTHSWSNDSDTVRSLKPWSQGTFSDQQWETLQHIFRVYRSSFTRDVKEFAKMLRLSYPLELQVSAGCEVHPGNASNNFFHVAFQGKDILSFQGTSWEPTQEAPLWVNLAIQVLNQDKWTRETVQWLLNGTCPQFVSGLLESGKSELKKQVKPKAWLSRGPSPGPGRLLLVCHVSGFYPKPVWVKWMRGEQEQQGTQPGDILPNADETWYLRATLDVVAGEAAGLSCRVKHSSLEGQDIVLYWGGSYTSGSLVPRGSGSKRVHHHHHHHH

SEQ ID NO: 117 - protein, 305 aa, Homo sapiens
  misc_feature: murine CD1d extracellular domain construct
QQKNYTFRCLQMSSFANRSWSRTDSVVWLGDLQTHRWSNDSATISFTKPWSQGKLSNQQWEKLQHMFQVYRVSFTRDIQELVKMMSPKEDYPIEIQLSAGCEMYPGNASESFLHVAFQGKYVVRFWGTSWQTVPGAPSWLDLPIKVLNADQGTSATVQMLLNDTCPLFVRGLLEAGKSDLEKQEKPVAWLSSVPSSADGHRQLVCHVSGFYPKPVWVMWMRGDQEQQGTHRGDFLPNADETWYLQATLDVEAGEEAGLACRVKHSSLGGQDIILYWDARQAPVGGSLVPRGSGSKRVHHHHHHHH

SEQ ID NO: 118 - protein, 303 aa, Homo sapiens
  misc_feature: mCD1dhu CD1d synthetic construct
QQKNYTFRCLQMSSFANRSWSRTDSVVWLGDLQTHRWSNDSATISFTKPWSQGKLSNQQWEKLQHMFQVYRVSFTRDIQELVKMLRLSYPLEIQLSAGCEMYPGNASESFLHVAFQGKYVVRFWGTSWQTVPGAPSWVNLAIKVLNADQGTSATVQMLLNDTCPLFVRGLLEAGKSDLEKQEKPVAWLSSVPSSADGHRQLVCHVSGFYPKPVWVMWMRGDQEQQGTHRGDFLPNADETWYLQATLDVEAGEEAGLACRVKHSSLGGQDIILYWDARQAPVGGSLVPRGSGSKRVHHHHHHHH

SEQ ID NO: 119 - protein, 305 aa, Homo sapiens
  misc_feature: hCD1dmu CD1d synthetic construct
EVPQRLFPLRCLQISSFANSSWTRTDGLAWLGELQTHSWSNDSDTVRSLKPWSQGTFSDQQWETLQHIFRVYRSSFTRDVKEFAKMMSPKEDYPIELQVSAGCEVHPGNASNNFFHVAFQGKDILSFQGTSWEPTQEAPLWVDLPIQVLNQDKWTRETVQWLLNGTCPQFVSGLLESGKSELKKQVKPKAWLSRGPSPGPGRLLLVCHVSGFYPKPVWVKWMRGEQEQQGTQPGDILPNADETWYLRATLDVVAGEAAGLSCRVKHSSLEGQDIVLYWGGSYTSGSLVPRGSGSKRVHHHHHHHH

SEQ ID NO: 120 - DNA, 19 bp, Homo sapiens
  misc_feature: F1
gtgcctgctgtttctgctg

SEQ ID NO: 121 - DNA, 20 bp, Homo sapiens
  misc_feature: R1
tgccctgataggaagtttgc

SEQ ID NO: 122 - protein, 6 aa, Homo sapiens
LSYPLE

SEQ ID NO: 123 - protein, 17 aa, Homo sapiens
QGTSWEPTQEAPLWVNL

SEQ ID NO: 124 - protein, 5 aa, Homo sapiens
DYAMH

SEQ ID NO: 125 - protein, 5 aa, Homo sapiens
GYYWS

SEQ ID NO: 126 - protein, 15 aa, Homo sapiens
DMCSSSGCPDGYFDS

SEQ ID NO: 127 - protein, 13 aa, Homo sapiens
GEIYDFWNSYMDV

SEQ ID NO: 128 - protein, 13 aa, Homo sapiens
GEIYDFWKSYMDV

SEQ ID NO: 129 - protein, 13 aa, Homo sapiens
GEIYDFWKSYLDV

SEQ ID NO: 130 - protein, 13 aa, Homo sapiens
GEIYDFYNSYMDV

SEQ ID NO: 131 - protein, 17 aa, Homo sapiens
TIIWNSAIIGYADSVKG

SEQ ID NO: 132 - protein, 16 aa, Homo sapiens
EINHSGSTNYNPSLKS

SEQ ID NO: 133 - protein, 16 aa, Homo sapiens
EINPSGSTNYNPSLKS

SEQ ID NO: 134 - protein, 16 aa, Homo sapiens
EINHAGSTNYNPSLKS

SEQ ID NO: 135 - protein, 7 aa, Homo sapiens
GFTFDDY

SEQ ID NO: 136 - protein, 7 aa, Homo sapiens
GGSFSGY

SEQ ID NO: 137 - protein, 6 aa, Homo sapiens
IWNSAI

SEQ ID NO: 138 - protein, 5 aa, Homo sapiens
NHSGS

SEQ ID NO: 139 - protein, 5 aa, Homo sapiens
NPSGS

SEQ ID NO: 140 - protein, 5 aa, Homo sapiens
NHAGS

SEQ ID NO: 141 - protein, 11 aa, Homo sapiens
RASQHISSWLA

SEQ ID NO: 142 - protein, 14 aa, Homo sapiens
ASSSGAVSSGNFPN

SEQ ID NO: 143 - protein, 9 aa, Homo sapiens
QQANRFPLT

SEQ ID NO: 144 - protein, 11 aa, Homo sapiens
LLYFGDTQLGV

SEQ ID NO: 145 - protein, 7 aa, Homo sapiens
AASSLQS

SEQ ID NO: 146 - protein, 7 aa, Homo sapiens
SASNKHS

SEQ ID NO: 147 - protein, 7 aa, Homo sapiens
LSYPLEL

SEQ ID NO: 148 - protein, 124 aa, Homo sapiens
  misc_feature (1)..(1): X is Q or E
XVQLVXSGGGLVXPGRSLRLSCAASGFTFDDYAMHWVRQAPGKGLEWVATIIWNSAIIGYADSVKGRFIVSRDNAKNSLYLQMNSLRAEDXALYYCAKDXCSSXGCPXGYFDSWGQGTLVTVSS

SEQ ID NO: 149 - protein, 108 aa, Homo sapiens
  misc_feature (45)..(45): X is N or K
DIQMTQSPSSVSASVGDRVTITCRASQHISSWLAWYQQKPGKAPXLLIYAASSLQSGVXSRFSGSGSGTDFTLTISSLQPEDFATYYCQQANRFPLTFGGGTKLEIKR

SEQ ID NO: 150 - protein, 121 aa, Homo sapiens
  misc_feature (13)..(13): X is R or K
QVQLQESGPGLVXPSETLSLTCAVYGGSFSGYYWSWIRQPPGKGLEWIGEINXXGSTNYNPSLKSRVTISVDTSKNQFSLKLSSVTAADTAVYYCARGEIYDFXXSYXDVWGKGTTVTVSS

SEQ ID NO: 151 - protein, 112 aa, Homo sapiens; identical to SEQ ID NO: 4

SEQ ID NO: 152 - protein, 99 aa, Homo sapiens
EVQLVESGGGLVQPGRSLRLSCAASGFTFDDYAMHWVRQAPGKGLEWVSGISWNSGSIGYADSVKGRFTISRDNAKNSLYLQMNSLRAEDTALYYCAKD

SEQ ID NO: 153 - protein, 124 aa, Homo sapiens
EVQLVESGGGLVKPGRSLRLSCAASGFTFDDYAMHWVRQAPGKGLEWVATIIWNSAIIGYADSVKGRFIVSRDNAKNSLYLQMNSLRAEDMALYYCAKDMCSSSGCPDGYFDSWGQGTLVTVSS

SEQ ID NO: 154 - protein, 124 aa, Homo sapiens; identical to SEQ ID NO: 24

SEQ ID NO: 155 - protein, 124 aa, Homo sapiens
QVQLVGSGGGLVKPGRSLRLSCAASGFTFDDYAMHWVRQAPGKGLEWVATIIWNSAIIGYADSVKGRFTISRDNAKNSLYLQMNSLRAEDMALYYCAKDMCSSSGCPDGYFDSWGQGTLVTVSS

SEQ ID NO: 156 - protein, 124 aa, Homo sapiens; identical to SEQ ID NO: 5

SEQ ID NO: 157 - protein, 124 aa, Homo sapiens; identical to SEQ ID NO: 1

SEQ ID NO: 158 - protein, 124 aa, Homo sapiens; identical to SEQ ID NO: 1

SEQ ID NO: 159 - protein, 124 aa, Homo sapiens; identical to SEQ ID NO: 153

SEQ ID NO: 160 - protein, 124 aa, Homo sapiens; identical to SEQ ID NO: 24

SEQ ID NO: 161 - protein, 124 aa, Homo sapiens; identical to SEQ ID NO: 155

SEQ ID NO: 162 - protein, 124 aa, Homo sapiens; identical to SEQ ID NO: 153

SEQ ID NO: 163 - protein, 124 aa, Homo sapiens; identical to SEQ ID NO: 155

SEQ ID NO: 164 - protein, 95 aa, Homo sapiens
DIQMTQSPSSVSASVGDRVTITCRASQGISSWLAWYQQKPGKAPKLLIYAASSLQSGVPSRFSGSGSGTDFTLTISSLQPEDFATYYCQQANSFP

SEQ ID NO: 165 - protein, 107 aa, Homo sapiens
DIQMTQSPSSVSASVGDRVTITCRASQHISSWLAWYQQKPGKAPNLLIYAASSLQSGVSSRFSGSGSGTDFTLTISSLQPEDFATYYCQQANRFPLTFGGGTKLEIK

SEQ ID NO: 166 - protein, 107 aa, Homo sapiens; identical to SEQ ID NO: 165

SEQ ID NO: 167 - protein, 107 aa, Homo sapiens; identical to SEQ ID NO: 165

SEQ ID NO: 168 - protein, 107 aa, Homo sapiens; identical to SEQ ID NO: 165

SEQ ID NO: 169 - protein, 107 aa, Homo sapiens
DIQMTQSPSSVSASVGDRVTITCRASQHISSWLAWYQQKPGKAPKLLIYAASSLQSGVSSRFSGSGSGTDFTLTISSLQPEDFATYYCQQANRFPLTFGGGTKLEIK

SEQ ID NO: 170 - protein, 107 aa, Homo sapiens
DIQMTQSPSSVSASVGDRVTITCRASQHISSWLAWYQQKPGKAPNLLIYAASSLQSGVPSRFSGSGSGTDFTLTISSLQPEDFATYYCQQANRFPLTFGGGTKLEIK

SEQ ID NO: 171 - protein, 107 aa, Homo sapiens; identical to SEQ ID NO: 169

SEQ ID NO: 172 - protein, 107 aa, Homo sapiens; identical to SEQ ID NO: 169

SEQ ID NO: 173 - protein, 107 aa, Homo sapiens; identical to SEQ ID NO: 169

SEQ ID NO: 174 - protein, 107 aa, Homo sapiens; identical to SEQ ID NO: 170

SEQ ID NO: 175 - protein, 107 aa, Homo sapiens; identical to SEQ ID NO: 170

SEQ ID NO: 176 - protein, 124 aa, Homo sapiens; identical to SEQ ID NO: 26

SEQ ID NO: 177 - protein, 124 aa, Homo sapiens
QVQLVGSGGGLVKPGRSLRLSCAASGFTFDDYAMHWVRQAPGKGLEWVATIIWNSAIIGYADSVKGRFIVSRDNAKNSLYLQMNSLRAEDTALYYCAKDICSSGGCPDGYFDSWGQGTLVTVSS

SEQ ID NO: 178 - protein, 124 aa, Homo sapiens
QVQLVGSGGGLVKPGRSLRLSCAASGFTFDDYAMHWVRQAPGKGLEWVATIIWNSAIIGYADSVKGRFIVSRDNAKNSLYLQMNSLRAEDTALYYCAKDLCSSGGCPDGYFDSWGQGTLVTVSS

SEQ ID NO: 179 - protein, 107 aa, Homo sapiens; identical to SEQ ID NO: 170

SEQ ID NO: 180 - protein, 107 aa, Homo sapiens
DIQMTQSPSSVSASVGDRVTITCRASQHISSWLAWYQQKPGKAPKLLIYAASSLQSGVPSRFSGSGSGTDFTLTISSLQPEDFATYYCQQANRFPLTFGGGTKLEIK

SEQ ID NO: 181 - protein, 107 aa, Homo sapiens; identical to SEQ ID NO: 180

SEQ ID NO: 182 - protein, 121 aa, Homo sapiens
QVQLQESGPGLVKPSETLSLTCAVYGGSFSGYYWSWIRQPPGKGLEWIGEINHAGSTNYNPSLKSRVTISVDTSKNQFSLKLSSVTAADTAVYYCARGEIYDFWKSYMEVWGKGTTVTVSS

SEQ ID NO: 183 - protein, 121 aa, Homo sapiens
QVQLQESGPGLVKPSETLSLTCAVYGGSFSGYYWSWIRQPPGKGLEWIGEINHAGSTNYNPSLKSRVTISVDTSKNQFSLKLSSVTAADTAVYYCARGEIYDFWKSYLEVWGKGTTVTVSS

SEQ ID NO: 184 - protein, 202 aa, Homo sapiens
KEFAKMLRLSYPLELQVSAGCEVHPGNASNNFFHVAFQGKDILSFQGTSWEPTQEAPLWVNLAIQVLNQDKWTRETVQWLLNGTCPQFVSGLLESGKSELKKQVKPKAWLSRGPSPGPGRLLLVCHVSGFYPKPVWVKWMRGEQEQQGTQPGDILPNADETWYLRATLDVVAGEAAGLSCRVKHSSLEGQDIVLYWGGSYTS

SEQ ID NO: 185 - protein, 206 aa, Mus musculus
QELVKMMSPKEDYPIEIQLSAGCEMYPGNASESFLHVAFQGKYVVRFWGTSWQTVPGAPSWLDLPIKVLNADQGTSATVQMLLNDTCPLFVRGLLEAGKSDLEKQEKPVAWLSSVPSSADGHRQLVCHVSGFYPKPVWVMWMRGDQEQQGTHRGDFLPNADETWYLQATLDVEAGEEAGLACRVKHSSLGGQDIILYWDARQAPVG

SEQ ID NO: 186 - protein, 204 aa, Homo sapiens
QELVKMLRLSYPLEIQLSAGCEMYPGNASESFLHVAFQGKYVVRFWGTSWQTVPGAPSWVNLAIKVLNADQGTSATVQMLLNDTCPLFVRGLLEAGKSDLEKQEKPVAWLSSVPSSADGHRQLVCHVSGFYPKPVWVMWMRGDQEQQGTHRGDFLPNADETWYLQATLDVEAGEEAGLACRVKHSSLGGQDIILYWDARQAPVG

SEQ ID NO: 187 - protein, 204 aa, Mus musculus
KEFAKMMSPKEDYPIELQVSAGCEVHPGNASNNFFHVAFQGKDILSFQGTSWEPTQEAPLWVNLAIQVLNQDKWTRETVQWLLNGTCPQFVSGLLESGKSELKKQVKPKAWLSRGPSPGPGRLLLVCHVSGFYPKPVWVKWMRGEQEQQGTQPGDILPNADETWYLRATLDVVAGEAAGLSCRVKHSSLEGQDIVLYWGGSYTS